SEQUENCE LISTING (35 sequences)

SEQ ID NO: 1 - DNA, 20 bp, Artificial Sequence
  Oligonucleotide
tatcctacagatgatgcgag

SEQ ID NO: 2 - DNA, 20 bp, Artificial Sequence
  Oligonucleotide
ctctcaagctgagtgggtcc

SEQ ID NO: 3 - DNA, 20 bp, Artificial Sequence
  Oligonucleotide
cagcactagcagtggcacgg

SEQ ID NO: 4 - DNA, 20 bp, Artificial Sequence
  Oligonucleotide
agacccagcagcactagcag

SEQ ID NO: 5 - DNA, 20 bp, Artificial Sequence
  Oligonucleotide
gagattgactatgatggcag

SEQ ID NO: 6 - DNA, 20 bp, Artificial Sequence
  Oligonucleotide
gctctgtctctcaagctgag

SEQ ID NO: 7 - DNA, 20 bp, Artificial Sequence
  Oligonucleotide
cagccactcgcatcatctgt

SEQ ID NO: 8 - DNA, 20 bp, Artificial Sequence
  Oligonucleotide
gctcagacacatcccaatcc

SEQ ID NO: 9 - DNA, 20 bp, Artificial Sequence
  Oligonucleotide
taggaaagccatcaccaagt

SEQ ID NO: 10 - DNA, 20 bp, Artificial Sequence
  Oligonucleotide
acgagcactcaccagcatcc

SEQ ID NO: 11 - DNA, 20 bp, Artificial Sequence
  Oligonucleotide
ctaggagtcagcgacatatg

SEQ ID NO: 12 - DNA, 20 bp, Artificial Sequence
  Oligonucleotide
gcagaagtccccggacacgt

SEQ ID NO: 13 - DNA, 20 bp, Artificial Sequence
  Oligonucleotide
gttcgagaccaacgtgtccg

SEQ ID NO: 14 - DNA, 20 bp, Artificial Sequence
  Oligonucleotide
cggggacttctgctacgttg

SEQ ID NO: 15 - DNA, 20 bp, Artificial Sequence
  Oligonucleotide
ccgctctgactcgctgcacg

SEQ ID NO: 16 - DNA, 20 bp, Artificial Sequence
  Oligonucleotide
ccccgtgcagcgagtcagag

SEQ ID NO: 17 - DNA, 66 bp, Artificial Sequence
  Oligonucleotide
tcaagctgagtgggtccgggctggggccaccaccgttgccgctgctagtgctgctggttctgaaaa

SEQ ID NO: 18 - DNA, 66 bp, Artificial Sequence
  Oligonucleotide
tcaagctgagtgggtccgggctggggccaccaccgtgccactgctagtgctgctgggtctggagat

SEQ ID NO: 19 - DNA, 75 bp, Artificial Sequence
  Oligonucleotide
  misc_feature (26)..(26): n is a, c, g, or t
tgggtccgggctggggccaccaccgntgccactgctagtgctgctgggtctggagatggtgagtaggagagactt

SEQ ID NO: 20 - DNA, 75 bp, Artificial Sequence
  Oligonucleotide
tgggtccgggctggggccaccaccgtgccactgctagtgctgctgggtctggagatggtgagtaggagagacttt

SEQ ID NO: 21 - DNA, 74 bp, Artificial Sequence
  Oligonucleotide
tgggtccgggctggggccaccaccggccactgctagtgctgctgggtctggagatggtgagtaggagagacttt

SEQ ID NO: 22 - DNA, 67 bp, Artificial Sequence
  Oligonucleotide
tgggtccgggctggggccaccaccgctagtgctgctgggtctggagatggtgagtaggagagacttt

SEQ ID NO: 23 - DNA, 64 bp, Artificial Sequence
  Oligonucleotide
tgggtccgggctggggccaccaccggtgctgctgggtctggagatggtgagtaggagagacttt

SEQ ID NO: 24 - DNA, 64 bp, Artificial Sequence
  Oligonucleotide
tgggtccgggctggggccactgctagtgctgctgggtctggagatggtgagtaggagagacttt

SEQ ID NO: 25 - DNA, 73 bp, Artificial Sequence
  Oligonucleotide
tgggtccgggctggggccaccactgccactgctagtgctgctgggtctggagatggtgagtaggagagacttt

SEQ ID NO: 26 - DNA, 64 bp, Artificial Sequence
  Oligonucleotide
tgggtccgggctggggccaccgctagtgctgctgggtctggagatggtgagtaggagagacttt

SEQ ID NO: 27 - DNA, 73 bp, Artificial Sequence
  Oligonucleotide
tgggtccgggctggggccaccaccgccactgctagtgctgctgggtctggagatggtgagtaggagagacttt

SEQ ID NO: 28 - DNA, 60 bp, Artificial Sequence
  Oligonucleotide
tcaagctgagtgggtccgggctggggccaccaccgtgccactgctagtgctgctgggtct

SEQ ID NO: 29 - DNA, 60 bp, Artificial Sequence
  Oligonucleotide
tcaagctgagtgggtccgggctggggccaccaccgttgccactgctagtgctgctgggtc

SEQ ID NO: 30 - DNA, 62 bp, Artificial Sequence
  Oligonucleotide
tcaagctgagtgggtccgggctggggccaccaccgttgccactgctagtgctgctgggtctg

SEQ ID NO: 31 - DNA, 60 bp, Artificial Sequence
  Oligonucleotide
agcaagcccatcccgtggctaggagtcagcgacatatggggagcacatctggttcgagac

SEQ ID NO: 32 - DNA, 60 bp, Artificial Sequence
  Oligonucleotide
agcaagcccatcccgtggctaggagtcagcgacattatggggagcacatctggttcgaga

SEQ ID NO: 33 - DNA, 61 bp, Artificial Sequence
  Oligonucleotide
agcaagcccatcccgtggctaggagtcagcgacattatggggagcacatctggttcgagac

SEQ ID NO: 34 - DNA, 20 bp, Artificial Sequence
  Oligonucleotide
ctcgatggtgaatgacagtg

SEQ ID NO: 35 - DNA, 20 bp, Artificial Sequence
  Oligonucleotide
gcttctgctgccggttaacg

(19) United States
(12) Patent Application Publication
(10) Pub. No.: US 2024/0117311 A1
(43) Pub. Date: Apr. 11, 2024

(54) METHODS AND COMPOSITIONS FOR GENERATING STEM CELL-DERIVED IMMUNE CELLS WITH ENHANCED FUNCTION

(71) Applicants: CARTHERICS PTY. LTD., Notting Hill (AU); TOOLGEN INCORPORATED, Seoul (KR)

(72) Inventors: Nicholas BOYD, Notting Hill (AU); Alan Osborne TROUNSON, Notting Hill (AU); Huimin CAO, Notting Hill (AU); Mathew James TIEDEMANN, Notting Hill (AU); Riehard BOYD, Notting Hill (AU); Runzhe SHU, Notting Hill (AU); Frederico Loureiro CALHABEU, Notting Hill (AU)

(21) Appl. No.: 18/276,794

(22) PCT Filed: Feb. 9, 2022
(86) PCT No.: PCT/AU2022/050075

§ 371 Date: Aug. 10, 2023

Related U.S. Application Data
(60) Provisional application No. 63/147,933, filed on Feb. 10, 2021.
(60) Provisional application No. 63/236,828, filed on Aug. 25, 2021.

(51) Int. Cl. C12N 5/0783; A61K 39/00; A61P 35/00; C12N 9/22

(52) U.S. Cl. CPC C12N 5/0646; A61K 39/4631; A61P 35/00; C12N 5/0636; C12N 9/22; C12N 2310/20; C12N 2506/45

(57) ABSTRACT

The instant disclosure is directed to methods for generating stem cell-derived immune cells with enhanced function. Disclosed herein are methods for modifying a stem or progenitor cell capable of differentiating into an immune cell to inhibit the function of at least one gene selected from DGKα and DGKζ, and directing differentiation of that stem or progenitor cells towards enhanced immune cells. Also disclosed herein are immune cells or stem cells made by the present methods, as well as the use of immune cells in therapeutic treatment.

### CROSS REFERENCE TO RELATED APPLICATION

This application claims the benefit of priority from U.S. Provisional Applications No. 63/147,933, filed Feb. 10, 2021, and 63/236,828, filed Aug. 25, 2021, the contents of which are incorporated herein in their entirety.

### FIELD OF THE DISCLOSURE

This disclosure relates to methods for generating stem cell-derived immune cells with enhanced function. More specifically, disclosed herein are methods for modifying a stem or progenitor cell capable of differentiating into an immune cell to inhibit the function of at least one gene selected from DGKα and DGKζ, and directing differentiation of that stem or progenitor cells towards enhanced immune cells. Also disclosed herein are immune cells or stem cells made by the present methods, as well as the use of immune cells in therapeutic treatment.

### INCORPORATION BY REFERENCE OF SEQUENCE LISTING

The Sequence Listing in the ASCII text file, named as 39095WO_SequenceListing.txt of 8 KB, created on Jan. 27, 2022, is incorporated herein by reference.

### BACKGROUND ART

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Malignant tumors, or cancers, grow in an uncontrolled manner, invade normal tissues, and often metastasize and grow at sites distant from the tissue of origin. In general, cancers are derived from one or only a few normal cells that have undergone a poorly understood process called malignant transformation. Cancers can arise from almost any tissue in the body. Those derived from epithelial cells, called carcinomas, are the most common kinds of cancers. Sarcomas are malignant tumors of mesenchymal tissues, arising from cells such as fibroblasts, muscle cells, and fat cells. Solid malignant tumors of lymphoid tissues are called lymphomas, and marrow and blood-borne malignant tumors of lymphocytes and other hematopoietic cells are called leukaemia.

Cancer is one of the three leading causes of death in industrialised nations. As treatments for infectious diseases and the prevention of cardiovascular disease continue to improve, and the average life expectancy increases, cancer is likely to become the most common fatal disease in these countries. Therefore, successfully treating cancer requires that all the malignant cells be removed or destroyed without killing the patient. An ideal way to achieve this would be to induce an immune response against the tumor that would discriminate between the cells of the tumor and their normal cellular counterparts.

Immunotherapy is a new area of treatment that has recently emerged, that uses the body's own immune system. This treatment may use cells such as T cell, NK cells, NKT cells, macrophages, B cells, dendritic cells, etc, obtained from living organisms or produced in the laboratory that are enhanced and injected to the patient's body to assists its immune system to fight cancer, infections and other diseases. Several types of immunotherapy are currently being developed.

Some of these treatments use T cells expressing chimeric antigen receptors (CAR-T cells) that have been shown to be very effective in killing tumor cells in diseases such as acute lymphocytic leukemia (ALL) and non-Hodgkin's lymphoma (NHL). Approved products targeting the B cell antigen CD19 are produced by introducing a CAR gene construct into patient-derived (“autologous”) T cells (Kershaw et al., Gene-engineered T cells for cancer therapy, Nat Rev Cancer, 2013, 13(8): 525-41). Additional autologous products are in development targeting other blood cell markers such as B cell maturation antigen (BCMA) for other haematological malignancies, such as multiple myeloma (Sadelain et al., Therapeutic T cell engineering, 2017, Nature, 545(7655): 423-431).

Other treatments have successfully used NK cells against haematological malignancies. Gene editing tools have been applied to NK and CAR-NK cells to enhance the therapeutic effect. Autonomous secretion or membrane bound expression of cytokines, like IL-15 and IL-2 could improve the NK cell expansion and persistence (Nagashima et al., Stable transduction of the interleukin-2 gene into human natural killer cell lines and their phenotypic and functional characterization in vitro and in vivo, Blood, 15 May 1998, Vol. 91(10), pp. 3850-61; Hoyos et al., Engineering CD19-specific T lymphocytes with interleukin-15 and a suicide gene to enhance their anti-lymphoma leukemia effects and safety, Leukemia, June 2010, Vol. 24(6), pp. 1160-70; Imamura et al., Autonomous growth and increased cytotoxicity of natural killer cells expressing membrane-bound interleukin-15, Blood, 14 Aug. 2014, Vol. 124(7), pp. 1081-8). Targeting immune checkpoints which were mostly studied in T cells, also showed promising results in NK cells. Knocking out TIGIT, an NK cell checkpoint receptor, has been shown to prevents the NK cell exhaustion and promote the anti-tumor activity (Zhang et al., Blockade of the checkpoint receptor TIGIT prevents NK cell exhaustion and elicits potent anti-tumor immunity, Nature Immunology, July 2018, Vol. 19(7), pp. 723-73). Moreover, gene disruption of receptors of immunosuppressive cytokines from the tumor microenvironment (TME), such as A2AR, TGFβR2 has been shown to neutralise the inhibition from TME and restore the NK cell effector function (Wang et al., SMAD4 promotes TGF-beta-independent NK cell homeostasis and maturation and antitumor immunity, The Journal of clinical investigation, 1 Nov. 2018, Vol. 128(11), pp. 5123-5136; Rouce et al., The TGF-beta SMAD pathway is an important mechanism for NK cell immune evasion in childhood B-acute lymphoblastic leukemia, Leukemia, April 2016, Vol. 30(4), pp. 800-811; Young et al., A2AR Adenosine Signaling Suppresses Natural Killer Cell Maturation in the Tumor Microenvironment, Cancer research, 15 Feb. 2018, Vol. 78(4), pp. 1003-1016).

Compared with T cells and CAR-T cells, NK cells and CAR-NK cell-based treatments present several advantages such as (1) better safety, particularly with respect to potentially life-threatening adverse events like cytokine release syndrome and graft versus host disease (2) multiple mechanisms for effecting cytotoxic activity, and (3) high feasibility for ‘off-the-shelf’ manufacturing (Xie et al., CAR-NK cells: A promising cellular immunotherapy for cancer, EBioMedicine, September 2020, Vol. 59; Liu et al., Use of CAR-Transduced Natural Killer Cells in CD19-Positive Lymphoid Tumors, The New England journal of medicine, Feb. 6, 2020, Vol. 382(6), pp. 545-553).

While cell-based immunotherapy has shown impressive results in clinical trial against blood-based cancers, similar results have not been forthcoming in the treatment of solid tumors. There are multiple reasons for the relative lack of efficacy in solid tumors, including restricted access to the tumor site, the immunosuppressive nature of the tumor microenvironment and the lack of solid tumor-specific target antigens.

In addition, lack of persistence and “exhaustion” of the administered CAR-cells is a consistently observed limitation (Newick et al., CAR T Cell Therapy for Solid Tumours, Annu Rev Med, 2017, 68: 139-152).

Inhibitory receptors like CTLA-4, PD-1, or LAG-3 can attenuate the activation of CAR-T cells and accelerate T cell exhaustion. An improved anti-tumor activity of T cells was expected after PD-1 was disrupted by genome editing (Liu et al., CRISPR-Cas9-mediated multiplex gene editing in CAR-T cells, Cell Res, 2017, 27(1): 154-157). However, ablation of PD-1 on T cells may also increase the susceptibility to exhaustion, reduce the longevity and fail to improve anti-tumor effect (Odorizzi et al., Genetic absence of PD-1 promotes accumulation of terminally differentiated exhausted CD8+ T cells, J Exp Med, 2015, 212(7): 1125-37). For these reasons, whether gene editing in immune cells will enhance anti-tumor activity or not needs to be evaluated case-by-case.

Engineered immune cells are clearly a potential weapon against cancer and therefore one of the challenges is to numerically generate, expand and retain them. Immune cells are created from hematopoietic stem cells (HSCs) that in turn are created from pluripotent stem cells (PSCs). PSCs technology is therefore a very promising technology as, theoretically, PSCs provide an unlimited renewable source of cells.

### SUMMARY OF THE DISCLOSURE

The present invention relates to compositions and methods for gene editing and differentiating cells, such as human PSCs into hematopoietic stem cell-like cells and ultimately into functional immune cells, where such immune cells possess benefits over equivalent non-gene edited immune cells. It will be appreciated by a person skilled in the art that deleting one or more non-redundant cellular genes may significantly affect normal cellular functions and, may in fact, render a cell non-viable, non-functional or, in the case of a stem cell, incapable of differentiation into a functional cell. The present invention discloses genetic knock-out (KO) of DGKα and/or DGKζ genes in iPSCs and, surprisingly, demonstrates that the DGK KO iPSCs are viable and can be differentiated into DGK KO immune cells, in particular NK and T cells. Furthermore, the DGK KO NK cells are fully functional in vitro and in vivo and, in fact, demonstrate superior function to NK cells that do not have the DGK KO. Similarly, the iPSCs-derived DGK KO T cells are fully functional in vitro and also exhibit improved function when compared to T cells that do not have the DGK KO.

In one aspect, provided herein is a method of generating stem cell-derived immune cells with enhanced function. The method comprises modifying stem cells to inhibit the function of at least one target gene selected from the group consisting of DGKα and DGKζ; wherein the modified stem cells are capable of differentiating into stem cell-derived immune cells that retain the target gene inhibition of the modified stem cells and comprise enhanced activity.

In some embodiments, the stem cells are pluripotent stem cells, selected from the group consisting of induced pluripotent stem cells (iPSCs) or embryonic stem cells. In some embodiments, the stem cells are selected from the group consisting of pre-HSCs, hemogenic endothelium (HE) or hematopoietic stem cells (HSCs) or HSC-like cells. In some embodiments, the stem cells are derived from a triple homozygous HLA haplotype donor.

In some embodiments, the method further comprises selecting the modified stem cells generated wherein both alleles of the target gene are inhibited.

In some embodiments, the method further comprises: (i) contacting the modified stem cell generated herein, or clonal cells generated from them, with a composition to obtain mesoderm cells; (ii) contacting the mesoderm cells with a composition to obtain CD34+ cells; and (iii) contacting the CD34+ cells with a composition to obtain stem cell-derived immune cells.

In some embodiments, the at least one target gene is DGKα. In some embodiments, the at least one target gene is DGKζ. In some embodiments, both DGKα gene and DGKζ gene are target genes.

In some embodiments, the stem cell-derived immune cells are selected from multipotent progenitor cells, lymphoid progenitor cells (e.g., common lymphoid progenitor cells), early thymic progenitor cells, pre-T cell progenitor cells, pre-NK progenitor cells, T progenitor cells, NK progenitor cells, myeloid progenitor cells (e.g., common myeloid progenitor cells), T cells, NK cells, NKT cells, B cells, macrophages and monocytes.

In some embodiments, the immune cells are T cells expressing at least one of the markers selected from CD2, CD5, CD7, CD4, CD8a, CD8b, CD3, TCRαβ and TCRγδ.

In some embodiments, the immune cells are NK cells expressing CD56+ and CD45+.

In some embodiments, wherein inhibition of the function of the target gene is achieved by a gene editing system. In some embodiments, the gene editing system is selected from the group consisting of CRISPR/Cas, TALEN and ZFN.

In some embodiments, the gene editing system is a CRISPR/Cas system which comprises a guide RNA-nuclease complex. In some embodiments, the guide RNA targets a nucleotide sequence selected from the group consisting of: SEQ ID NO: 1 to SEQ ID NO: 16.

In some embodiments, the CRISPR/Cas system utilizes a guide RNA dependent nuclease selected from the group consisting of Cpf1, Cas1, Cas1B, Cas2, Cas3, Cas4, Cas5, Cas6, Cas7, Cas8, Cas9, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, and Csf4.

In some embodiments, the modified stem cells are further modified to comprise a nucleic acid encoding a chimeric antigen receptor (CAR). In some embodiments, the modified stem cells express the CAR.

In some embodiments, the immune cells produced by the method are modified to comprise a nucleic acid encoding a chimeric antigen receptor (CAR). In some embodiments, the immune cells produced by the method express the CAR.

In some embodiments, the immune cells produced by the method recognize one or more target antigens. In some embodiments, the immune cells produced by the method recognize a tumor target or an infectious agent target. In some embodiments, the target antigens are selected from the group consisting of TAG-72, CCR4, CD19, CD20, CD22, CD24, CD30, CD47, folate receptor alpha (FRα), BCMA, mesothelin, Muc1.

In a further aspect, provided herein is an immune cell produced by a method disclosed herein.

In another aspect, provided herein is a modified cell, wherein the function of at least one target gene selected from the group consisting of DGKα and DGKζ is inhibited, wherein the modified cell is capable of differentiating to an immune cell that retains the gene inhibition of the modified cell and comprises enhanced activity.

In some embodiments, the modified cell is a stem cell selected from the group consisting of embryonic stem cells, umbilical cord stem cells and induced pluripotent stem cells. In some embodiments, wherein the modified cell is selected from the group consisting of a hemogenic endothelium cell, hematopoietic progenitor cell, hematopoietic precursor cell, hematopoietic stem cell or hematopoietic-like stem cell. In some embodiments, the modified cell is derived from a triple homozygous HLA haplotype donor.

In some embodiments, both alleles of the target gene are inhibited. In some embodiments, the at least one target gene is DGKα. In some embodiments, the at least one target gene is DGKζ. In some embodiments, both DGKα and DGKζ genes are target genes.

In some embodiments, the modified cell comprises a nucleic acid encoding a chimeric antigen receptor (CAR). In some embodiments, the modified cell expresses the CAR.

In another aspect, provided herein is a composition for modifying a cell to inhibit the function of at least one target gene selected from the group consisting of DGKα and DGKζ comprising: a guide RNA-nuclease complex capable of editing the sequence of a target gene, wherein the guide RNA targets a nucleotide sequence is selected from the group consisting of: SEQ ID NO: 1 to SEQ ID NO: 16.

In some embodiments, the nuclease comprises at least one protein selected from the group consisting of Cpf1, Cas1, Cas1B, Cas2, Cas3, Cas4, Cas5, Cas6, Cas7, Cas8, Cas9, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, and Csf4.

In a further aspect, provided herein is a method of treating a condition in a subject, comprising administering to the subject an immune cell disclosed herein. In some embodiments, the condition is a cancer, an infection, an autoimmune disorder, fibrosis of an organ, or endometriosis.

### BRIEF DESCRIPTION OF THE DRAWINGS

This patent or application file contains at least one drawing executed in colour. Copies of this patent or patent application publication with colour drawing(s) will be provided by the Office upon request and payment of the necessary fee.

FIG. 1. A schematic of iPSC gene editing and single-cell cloning. CRISPR/Cas9 gene editing was used to knock-in (KI) TAG-72 CAR (as described in patents PCT/AU2020/050800 and WO2017/088012, incorporated herein by reference) or knock-out (KO) DGK genes (DGKα and/or DGKζ) in iPSCs or KO DGK genes (DGKα and/or DGKζ) in CAR iPSC clones. iPSCs or CAR iPSC clones were allowed to recover from electroporation until the iPSC are growing normally again. This is typically anytime beyond 4-6 days following electroporation. Upon reaching 80% colony confluency, iPSCs were expanded until day 11 before single cell sorting (Phase I). Non-transfected iPSCs are referred to as wildtype. Edited iPSCs or CAR iPSC clones at the end of phase I are termed in the following examples as pre-sorted. Single cell sorting of iPSCs was performed using the FACSAria™ Fusion cell sorter and colony formation from single cells was allowed to progress for 9 to 12 days. Following this, clones were expanded until the required cell number was reached (Phase II) and genetic analysis was carried out to determine accuracy and purity in clonal iPSCs. iPSCs at the end of phase II are termed in the following examples as single-cell clones.

FIGS. 2A-2C. CRISPR/Cas9 introduces insertions and deletions (indels) into the open reading frame of the DGKα gene in iPSCs. A guide RNA (gRNA) targeting target sequence of DGKα gene (SEQ ID NO: 3) formed ribonucleoproteins (RNPs) were transfected into iPSCs and the frequency of indels was assessed by Inference of CRISPR Edits (ICE) analysis. (A) Sanger sequencing traces from a gRNA targeting target sequence of DGKα gene (SEQ ID NO: 3) RNP transfected iPSCs (“edited sample”) (SEQ ID NO: 17) and non-transfected iPSCs (“control sample”) (SEQ ID NO: 18) are shown. The edited sample shows a heterogeneous mix of bases downstream of the cut site in contrast to the non-transfected control sample. The black underlined region of the control sample represents the guide sequence and the horizontal red dotted underlined region is the associated (protospacer adjacent motif) PAM site. The vertical black dotted lines on both traces represent the cut site. (B) Relative percentage of the contribution of each edited sequence (normalized) in the genomic DNA from a gRNA targeting target sequence of DGKα gene (SEQ ID NO: 3) RNP transfected iPSCs. Indel +1: SEQ ID NO: 19; Indel 0: SEQ ID NO: 20; Indel −1: SEQ ID NO: 21; Indel −8: SEQ ID NO: 22; Indel −11: SEQ ID NO: 23; Indel −11: SEQ ID NO: 24; Indel −2: SEQ ID NO: 25; Indel −11: (SEQ ID NO: 26); Indel −2: SEQ ID NO: 27. (C) Distribution of the indel sizes in the entire edited population of a gRNA targeting target sequence of DGKα gene (SEQ ID NO:3) RNP transfected iPSCs. Out-of-frame indel percentage is the proportion of indels that indicate a frameshift or are more than 21 bp in length. The R² value computed by the Pearson correlation coefficient indicates the confidence of the indel percentage.

FIGS. 3A-3B. CRISPR/Cas9 editing of DGK genes is successfully mediated in several iPSC lines. A gRNA targeting target sequence of DGKα gene (SEQ ID NO: 3) and a gRNA targeting target sequence of DGKζ gene (SEQ ID NO: 11) formed RNPs were transfected into two different iPSC lines delivered from different cell types and using different methods from two independent companies: iPSC line 1 (A) or iPSC line 2 (B) to generate DGKα or DGKζ single KO (DGKα KO or DGKζ KO iPSCs), or DGKα and DGKζ double KO (DGKαζ KO iPSCs). iPSC line1 was derived from mononuclear cells using episomal delivery of iPSC reprogramming factors. iPSC line2 was derived from cells obtained from a cord blood fraction and reprogrammed using Sendai viral delivery of iPSC factors. KO efficiency of DGKα and DGKζ genes were analysed using ICE. Out-of-frame indel percentage is the proportion of indels that indicate a frameshift or are more than 21 bp in length. The R² value computed by Pearson correlation coefficient indicates the confidence of the indel percentage.

FIGS. 4A-4D. gRNA mediates successful CRISPR/Cas9 editing of DGK genes in single-cell clones. A gRNA targeting target sequence of DGKα gene and a gRNA targeting target sequence of DGKζ gene (SEQ ID NO: 3 and SEQ ID NO: 11 respectively) formed RNPs were transfected into iPSCs and the efficiencies of DGKα KO (A), DGKζ KO (B) and DGKαζ KOs DGKα indel % (C), and DGKζ indel % (D) were analysed using ICE in different single-cell clones. Out-of-frame indel percentage is the proportion of indels that indicate a frameshift or are more than 21 bp in length. iPSC single-cell clones with 99% to 100% out-of-frame indel percentage were selected as single-cell clone candidates. Clone names are designated as the gene name followed by a number.

FIGS. 5A-5B. Sanger sequencing traces of gRNA cut sites in DGK genes in non-transfected (wildtype), DGKαζ KO pre-sorted iPSCs and in DGKαζ KO iPSCs single-cell clone 01. After the RNP transfection the mix of bases were introduced to the downstream of the cut site in the DGK KO pre-sorted iPSCs genome. After sorting and single-cell cloning, a single-cell iPSC clone (DGKαζ KO iPSC single-cell clone 01) with one thymine (T) insertion (+1) was identified from the DGK KO pre-sorted iPSCs which is evidenced by removing the mix of bases downstream of the cut site in the DGKα (A) and DGKζ (B) gene. The black underlined region of the control sample represents the guide sequence and the horizontal red dotted underlined region is the associated PAM site. The vertical black dotted line on both traces represents the cut site. In (A), the following sequences are shown: Non-transfected (wildtype) iPSC: SEQ ID NO: 28; DGKα and DGKζ RNP transfected bulk iPSC: SEQ ID NO: 29; DGKα and DGKζ RNP co-transfected iPSC single-cell clone: SEQ ID NO: 30. In (B), the following sequences are shown: Non-transfected (wildtype) iPSC: SEQ ID NO: 31; DGKα and DGKζ RNP transfected bulk iPSC: SEQ ID NO: 32; DGKα and DGKζ RNP co-transfected iPSC single-cell clone: SEQ ID NO: 33.

FIG. 6. CRISPR/Cas9 editing of DGK genes in TAG-72 CAR iPSC single-cell clones is successfully mediated. TAG-72 CAR iPSC single-cell clones were generated as described in FIG. 1. Subsequently, a gRNA targeting target sequence of DGKα gene and a gRNA targeting target sequence of DGKζ gene (SEQ ID NO: 3 and SEQ ID NO: 11 respectively) formed RNPs were transfected into three different TAG-72 CAR iPSC single-cell clones (TAG-72 CAR iPSC single-cell clones B11, C4 and D7) to generate TAG-72 CAR clones/DGKαζ KO iPSCs. KO efficiency of DGKα and DGKζ genes KO in TAG-72 CAR clone/DGKαζ KO pre-sorted iPSCs were individually analysed using ICE. Out-of-frame indel percentage is the proportion of indels that indicate a frameshift or are more than 21 bp in length. The R² value computed by Pearson correlation coefficient indicates the confidence of the indel percentage.

FIG. 7. Successful generation of TAG-72 CAR/DGKαζ KO iPSC single-cell clones. TAG-72 CAR iPSC single-cell clones were generated as described in FIG. 1. Subsequently, a gRNA targeting target sequence of DGKα gene and a gRNA targeting target sequence of DGKζ gene (SEQ ID NO: 3 and SEQ ID NO: 11 respectively) formed RNPs were transfected into TAG-72 CAR iPSC single-cell clone B11 and TAG-72 CAR/DGKαζ KO iPSC single-cell clones (1-12) were generated. KO efficiency of DGKα and DGKζ genes were individually analysed using ICE. Out-of-frame indel percentage is the proportion of indels that indicate a frameshift or are more than 21 bp in length. Clones with 99% to 100% out-of-frame indel percentage were selected as single-cell clone candidates. Numbers 1-12 represent TAG-72 CAR/DGKαζ KO iPSC single-cell clones and those highlighted represent successful KO.

FIGS. 8A-8B. Gene deletion of DGKα and/or DGKζ in iPSCs does not affect iPSC pluripotency. This was characterised by (A) colony morphology (scale bars=1 mm), and (B) surface expression of pluripotent markers TRA-1-60 and SSEA-4. TAG-72 CAR KI, single gene (DGKα or DGKζ) and double gene (DGKαζ) KOs of DGK isoforms in iPSCs were carried out using CRISPR/Cas9 editing. Pluripotent stem cell markers (TRA-1-60 and SSEA-4) were analysed by flow cytometry, where the histograms (FIG. 8B) represent the expression of each pluripotent marker on all live cells (dead cells via PI, debris and doublet were gated out). Unstained controls are included in the histograms to highlight positive antibody staining.

FIGS. 9A-9B. Gene deletion of DGKα and/or DGKζ and TAG-72 CAR insertion into iPSCs does not impact the growth of iPSCs compared to the non-transfected (NT) control. The graphs represent a time course of iPSCs expansion. (A) Direct comparison between each allele KO. (B) DGKαζ KO performed on two different CAR iPSC single-cell clones; both with respect to the non-transfected iPSCs (NT) line. (A): Blue line (NT) represents non-transfected iPSCs; red line (DGKα) represents DGKα KO iPSC single-cell clone 16; green line (DGKζ) represents DGKζ KO iPSC single-cell clone 02; purple line represents DGKα KO iPSC single-cell clone 24. (B): Blue line (NT) represents non-transfected iPSCs; red line TAG-72 CAR (clone D7) represents TAG-72 CAR iPSC single-cell clone D7 (without DGKα and/or DGKζKO); green line TAG-72 CAR clone/DGKαζ KO (clone D7) represents TAG-72 CAR clone D7/DGKαζ KO iPSCs pre-sorted.

FIG. 10. Schematic of iPSC to iNK differentiation method. iPSCs were lifted and differentiated toward hemogenic/hematopoietic linage in phase 1. CD34+ cells were then differentiated toward lymphoid progenitor cells in phase 2 and directed toward iNK cells in phase 3.

FIGS. 11A-11B. TAG-72 CAR KI and DGK gene KO does not impact differentiation of iPSCs to CD34+ cells. TAG-72 CAR KI, single gene (DGKα or DGKζ) or double gene (DGKαζ) KOs of DGK isoforms in iPSCs was carried out using CRISPR/Cas9 editing. iPSCs were subsequently differentiated to CD34+ cells and analysed by flow cytometry. Representative flow cytometry plots show the population frequencies of CD34+ cells in the absence (A) or presence (B) of TAG-72 CARs. A: iPSC (Non-transfected) represents CD34+ cells derived from non-transfected iPSCs; DGKα KO (clone 16) represents CD34+ cells derived from DGKα KO iPSC single-cell clone 16; DG (KO (clone 07) represents CD34+ cells derived from DGKζ KO iPSC single-cell clone 07; DGKα KO (clone 24) represents CD34+ cells derived from DGKα KO iPSC single-cell clone 24. B: iPSC (Non-transfected) represents CD34+ cells derived from non-transfected iPSCs in this experiment; TAG-72 CAR (clone D7) represents CD34+ cells derived from TAG-72 CAR iPSC single-cell clone (clone D7); TAG-72 CAR (clone D7)/DGKαζ KO pre-sorted represents CD34+ cells derived from TAG-72 CAR clone D7/DGKαζ KO pre-sorted iPSCs. The histogram representation of the cell surface expression of CD34 is on live cells in culture. Dead cells, debris and doublets were gated out. Unstained controls (blue) are included in the histograms to highlight positive antibody staining.

FIGS. 12A-12B. CRISPR/Cas9 mediates the KO of DGK genes in iNKs with and without TAG-72 CAR. TAG-72 CAR iPSC single-cell clones were generated as described in FIG. 1. A gRNA targeting target sequence of DGKα gene (SEQ ID NO: 3) and a gRNA targeting target sequence of DGKζ gene (SEQ ID NO: 11) formed RNPs were used to generate TAG-72 CAR clone/DGKαζ KO pre-sorted iPSCs (A). Similarly, a gRNA targeting target sequence of DGKα gene (SEQ ID NO: 3) and a gRNA targeting target sequence of DGKζ gene (SEQ ID NO: 11) formed RNPs were used to generate DGKαζ KO iPSCs and DGKα KO iPSC single-cell clones were formed as described in FIG. 1 (B). TAG-72 CAR clone D7/DGKαζ KO pre-sorted iPSCs and DGKαζ KO iPSC single-cell clone 24 were then differentiated into iNK cells. KO efficiency of DGKα and DGKζ genes in the iNK cells were analysed using ICE. Out-of-frame indel percentage is the proportion of indels that indicate a frameshift or are more than 21 bp in length.

FIG. 13. TAG-72 CAR KI and DGK gene KO does not impact the number of iNK cells generated per iPSC. TAG-72 CAR KI, single gene (DGKα or DGKζ) or double gene (DGKαζ) KO of DGK isoforms in iPSCs was carried out using CRISPR/Cas9 editing as previously discussed. iPSCs were then differentiated into iNK cells. Representative yield of iNK cells (CD56+ cells) differentiated from KO iPSC single-cell clones with and without the inclusion of a CAR construct, compared to the baseline yield of iNK cells generated from non-transfected iPSCs is represented as the mean±standard deviations (SD) from 2-5 samples, i.e. a representative yield score of 1 in any of the gene-edited samples, indicates this sample has generated the same number of iNK cells that is from non-transfected iPSC, and therefore the gene-edit has no impact on iNK yield. iNK (Non-transfected) represents iNKs derived from non-transfected iPSCs; iNK DGKα KO represents iNKs derived from DGKα KO iPSC single-cell clones; iNK DGKζ KO represents iNKs derived from DGKζ KO iPSC single-cell clones; iNK DGKαζ KO represents iNKs derived from DGKαζ KO iPSC single-cell clones; iNK TAG-72 CAR represents iNKs derived from TAG-72 CAR iPSC single-cell clones; iNK TAG-72 CAR/DGKαζ KO represents iNKs derived from TAG-72 CAR clones/DGKαζ KO iPSCs pre-sorted. One-way ANOVA statistics were performed across all groups. No significant differences were identified.

FIGS. 14A-14B. TAG-72 CAR KI and DGK gene KO does not alter phenotype of iNKs. TAG-72 CAR KI, single gene (DGKα or DGKζ) or double gene (DGKαζ) KO of DGK isoforms in iPSCs was carried out using CRISPR/Cas9 editing. iPSCs were then differentiated into iNK cells and phenotype was assessed by flow cytometry. Representative phenotypic analysis of iNKs differentiated from KO iPSCs in the absence (A) or presence (B) of TAG-72 CARs. For flow analysis, dead cells, debris and doublets have been gated out, such that each histogram represents all live cells in culture. Unstained controls (blue) are included in the histograms to highlight positive antibody staining for NK receptors CD56, CD45, NKp46, NKG2D, Nkp44 and 2B4. iNK (Non-transfected) represents iNK derived from non-transfected iPSCs; iNK DGKα KO (clone 16) represents iNK derived from DGKα KO iPSC single-cell clone 16; iNK DGKζ KO (clone 07) represents iNK derived from DGKζ KO iPSC single-cell clone 07; iNK DGKαζ KO (clone 24) represents iNK derived from DGKα KO iPSC single-cell clone 24; iNK TAG-72 CAR (clone D7) represents iNK derived from TAG-72 CAR iPSC single-cell clone D7; iNK TAG-72 CAR (clone D7)/DGKαζ KO (pre-sorted) represents iNK derived from TAG-72 CAR clone D7/DGKαζ KO pre-sorted iPSCs; iNK TAG-72 CAR (clone C4) represents iNK derived from TAG-72 CAR iPSC single-cell clone C4; iNK TAG-72 CAR (clone C4)/DGKαζ KO (pre-sorted) represents iNK derived from TAG-72 CAR (clone C4)/DGKαζ KO pre-sorted iPSCs.

FIG. 15A-15C. TAG-72 CAR KI and DGK gene KO enhance iNK cell cytotoxicity against OVCAR-3 tumor cells, as well as enhanced survival/longevity following repeated exposure to the same cells. TAG-72 CAR KI and double gene (DGKαζ) KO of DGK isoforms in iPSCs was carried out using CRISPR/Cas9 editing. iPSCs were then differentiated into iNK cells and the cytotoxicity against OVCAR-3 cells was determined using the real-time cell monitoring system (xCELLigence®). Survival/longevity of iNK cells was assessed in repeat antigen exposure assays. Cytotoxic function of TAG-72 CAR iNK cells, with and without DGKαζ KO, is shown as killing efficiency of OVCAR-3 cells over 72 hrs (A). Killing efficiency was calculated as a proportion of OVCAR-3 cells remaining following treatment relative to un-treated OVCAR-3 controls. In addition, cytotoxic function of pre-exposed iNK cells to OVCAR-3 cells was assessed using xCELLigence and presented as normalized cell index over 20 hrs (B). Normalized cell index was calculated following manufacturer's recommendations. (C) The number of iNK cells remaining in culture after 72 hrs repeat exposure to OVCAR-3 cells is also shown. iNK TAG-72 CAR (clone C4) represents iNK cells derived from TAG-72 CAR iPSC single-cell clone (clone C4); iNK TAG-72 CAR (clone C4)/DGKαζ KO (pre-sorted) represents iNK cells derived from TAG-72 CAR (clone C4)/DGKαζ KO pre-sorted iPSCs; iNK TAG-72 CAR (clone D7) represents iNK cells derived from TAG-72 CAR iPSC single-cell clone D7; iNK TAG-72 CAR (clone D7)/DGKαζ KO (pre-sorted) represents iNK cells derived from TAG-72 CAR (clone D7)/DGKαζ KO pre-sorted iPSCs.

FIGS. 16A-16B. DGK αζ KO iNK cells demonstrate improved killing capacity compared to non-transfected iNK controls. (A) Normalized cell index (CI) readings over 24 hrs for DGK αζ KO and non-transfected iNK cells, as measured by xCELLigence® real-time cell monitoring system. Error bars=SEM, NGM=normal growth medium for OVCAR-3 cells. (B) Expression of the pro-proliferative marker Ki67 is increased on DGKαζ KO iNK cells following 120 hrs of repeated antigen stimulation with OVCAR-3 cells or left in culture under normal NK growth conditions. The proportion of Ki67+CD56+ positive cells as determined by flow cytometry at the end of 120 hrs repeated antigen stimulation. Error bars=SEM.

FIG. 17. The inclusion of DGKαζ KO in iNK cells provides a function advantage in vivo compared to non-transfected iNK cells (iNK cells without the DGKαζ KO), in reducing tumour size and prolonging the survival of nod-skid-gamma (NSG) mice carrying subcutaneous ovarian cancer (OVCAR-3) tumors. iNK cells (with and without the DGKαζ KO) were injected 3×10⁶ cells spaced 14 days apart. The dotted lines represent when each iNK injection was performed. The iNK cells were recovered in vitro in NK growth media prior to injection for 7 days. No cytokine support was co-administered during the in vivo experiment. (n=4 mice per group).

FIGS. 18A-18C. DGKαζ gene deletion renders iNK resistance to TGFß immune suppression. (A) iNK cells with and without DGKαζ KO targeted against ovarian cancer cells (OVCAR-3) in vitro, were assessed for their cytotoxic function via xCELLigence in culture media with or without the addition of TGFß at 10 ng/mL. (B) The Normalized cell index (CI) readings as measured by xCELLigence® real-time cell monitoring system demonstrated at 2:1 (effector:target) ratio, that the addition of TGFβ inhibited non-transfected iNK cell function, but did not suppress iNK function in cells containing the DGKαζ KO. xCELLigence curves represent average+standard deviation (n=3). (C) TAG-72 CAR (clone D7) iNK cells with or without DGKαζ KO were assessed for their cytotoxic function against ovarian cancer cells (OVCAR-3) in vitro in culture media with or without the addition of TGFß at 100 ng/mL via xCELLigence. xCelligence® curves represent average+standard deviation (n=3).

FIG. 19. TGFβR1/2 gene editing results in loss of pluripotency and spontaneous differentiation of iPSCs. iPSC colony morphology was examined by light microscopy after RNP transfection. In contrast to the non-transfected (NT) iPSCs, TGFβR1 and TGFβR2 dominant negative knockout iPSC colonies displayed spontaneous differentiation after targeted transfection.

TGFβR KO gRNA

| | |
|---|---|
| TGFβR1 | CTCGATGGTGAATGACAGTG (SEQ ID NO: 34) |
| TGFβR2 | GCTTCTGCTGCCGGTTAACG (SEQ ID NO: 35) |

FIGS. 20A-20C. In vitro characterization of phenotype and function of gene edited iNK cells. (A) Flow cytometric analysis of different iNK effector populations prior to in vivo administration. CAR and NK surface receptor expression is reflected as a percentage of the CD45+ CD56+ double-positive population. (B) ICE analysis of DGKα and ζ loci as characterized by in-del % performed on iNK cells generated from enriched TAG-72 CAR iPSC lines with DGKαζ KO (non-clonally derived) (n=2). (C) iNK functional killing assay: effectors were added to OVCAR-3 target cells at E:T ratios of 1:2 and 1:1, and monitored for 20 hrs using an xCELLigence® system. NCI=Normalized cell index.

FIGS. 21A-21E. In vivo function of TAG-72 CAR and TAG-72 CAR/DGKαζ iNK cells. (A, B) Representative bioluminescence images (BLI) of immunocompromised NSG mice bearing Luciferase-labelled OVCAR-3 tumor cells from two independent experiments. Images reflect several time-points post administration of freeze-thawed NK cells as indicated. (C) Quantification of tumor burden plotted as fold change of flux units (photons/sec) over time, relative to day −1 baseline BLI reading for each group of mice (shown as the median BLI for each group). (D, E) Histograms showing individual mouse BLI signals (plotted as fold change over baseline) at each timepoint post-treatment. n=4-5 mice per group, 3 independent experiments. Error bars represent mean±SEM for each group.

FIG. 22A-22C. iPSC gene-edited with TAG-72 CAR+DGKαζ differentiated into iT cells. (A) Indel efficiency of DGKα and DGKζ in TAG-72 CAR iPSC by TIDE analysis. (B) Flow cytometric analysis of T cell markers and CAR expression, comparing non-edited iT cells with TAG-72 CAR+DGKαζ iT cells (non-clonally derived). (C) Cytotoxic functional analysis of T cells isolated from the peripheral blood mononuclear cells PBMC) fraction of healthy donors, compared to iT (non-edited) and iT cells differentiated from TAG-72 CAR+DGKαζ KO iPSC targeted against the ovarian cancer cell line OVCAR-3 (which expresses TAG-72).

FIG. 23A-23C. In vitro function of TAG-72 CAR iT/DGKαζ KO cells in the presence of TGFβ. (A) Schematic representation of the methodology implemented to assess the impact of TGFβ pre-conditioning on iT cell function in vitro. (B-C) iT cell in vitro cytotoxicity assay: effectors±TGFβ (B: 10 ng/mL, C: 100 ng/mL) were added to OVCAR-3 targets at an E:T ratio of 1:1 and monitored for 40 h using the real time cell monitoring system, xCELLigence®. The function of TAG-72 CAR-iT/DGKαζ KO cells (purple) were compared to iT (non-transfected) controls (grey). Target cells alone±TGFβ (blue) were maintained and monitored in parallel, providing growth kinetics of the OVCAR-3 cell line in the absence of effectors. Data are normalised to the time of addition of effector cells and presented as the arbitrary unit, Normalised cell index. Each data point represents the average±SD of technical triplicates.

### DETAILED DESCRIPTION

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Through the specification and claims the terms “a” and “an” are to be taken to mean “at least one” and are not to be taken as excluding “two or more” unless the context clearly dictates otherwise.

A “nucleic acid construct”, as used herein, generally refers to a nucleic acid molecule that is constructed or made artificially or recombinantly, and is also interchangeably referred to as a nucleic acid vector. For example, a nucleic acid construct can be made to include a nucleotide sequence of interest that is desired to be transcribed in a cell, and in some instances, to produce a RNA molecule of a desired function (e.g., an antisense RNA, siRNA, miRNA, or gRNA), and in other instances, to produce an mRNA which is translated into a protein of interest (e.g., a Cas protein). The nucleotide sequence of interest in a nucleic acid construct can be operably linked to a 5′ regulatory region (e.g., a promoter such as a heterologous promoter), and/or a 3′ regulatory region (e.g., a 3′ untranslated region (UTR) such as a heterologous 3′ UTR). The nucleic acid construct can be in a circular (e.g., a plasmid) or linear form, can be an integrative nucleic acid (i.e., capable of being integrated into the chromosome of a host cell, e.g., a viral vector such as a lentiviral vector) or can remain episomal (e.g., a plasmid).

The terms “about” or “approximately” should be understood as ±10% variation from a given value.

The term “ex vivo” a process in which cells are removed from a living organism and are propagated outside the organism (e.g., in a test tube).

The term “in vivo” refers to events that occur within an organism (e.g. animal, plant, and/or microbe).

Unless otherwise defined, all technical and scientific terms used herein have the same meaning as commonly understood by one of ordinary skill in the art to which this invention belongs.

### General Description

Disclosed herein are methods for generating stem cell-derived immune cells with enhanced function. For example, it has been demonstrated herein that ablation of one or more selected genes in stem cells using CRISPR/Cas9 gene editing does not disrupt the stem cells' capability of differentiating towards immune cells. It has also been demonstrated that the immune cells generated from these modified stem cells comprise enhanced persistence, anti-tumor activity in vitro and resistance to immunosuppressive effects of TGFβ. Accordingly, methods are provided by inhibiting the function of one or more selected genes in stem cells. In addition, methods are provided to differentiate theses stem cells towards immune cells, such as NK cells and T cells. Also, disclosed herein are modified stem cells and generated immune cells by the present methods, as well as the use of immune cells in therapeutic treatment.

### Source Cells

A “source cell”, as used herein, refers to the cell to be converted to a “derived cell” by reprogramming or differentiation. Examples of source cells suitable for use in the methods disclosed herein include stem cells.

The term “stem cell” should be understood as a reference to any cell which exhibits the potentiality to develop in the direction of multiple lineages, given its particular genetic constitution, and thus to form a new organism or to regenerate a tissue or cellular population of an organism. The stem cells which are utilized in accordance with the present invention may be of any suitable type capable of differentiating along two or more lineages and include, but are not limited to, embryonic stem cells (ESCs), adult stem cells, umbilical cord stem cells, haemopoietic stem cells (HSCs), progenitor cells, precursor cells, pluripotent cells, multipotent cells or de-differentiated somatic cells (such as an induced pluripotent stem cell). By “pluripotent” is meant that the subject stem cell can both self renew and differentiate to form, inter alia, cells of any one of the three germ layers, these being the ectoderm, endoderm and mesoderm.

In some embodiments, the source cell, also expresses at least one homozygous HLA haplotype. In some embodiments, a source cell expresses at least one homozygous HLA haplotype which is a major transplantation antigen and which is preferably expressed by a significant proportion of the population, such as at least 5%, at least 10%, at least 15%, at least 17%, at least 20%, or more of the population. Where the homozygous HLA haplotype corresponds to a dominant MHC I or MHC II HLA type (in terms of tissue rejection), the use of such a cell will result in significantly reduced problems with tissue rejection in the wider population who receive the cells of the present invention in the context of a treatment regime.

In other embodiments, a source cell may be homozygous in relation to more than one HLA antigen, e.g., two, three, or more HLA antigens. HLA antigens of interest can be selected from e.g., HLA A1, B8, C7, DR17, DQ2, or HLA A2, B44, C5, DR4, DQ8, or HLA A3, B7, C7, DR15, DQ6.

In some embodiments, the source cell is homozygous in relation to the inhibited gene.

In some embodiments, a source cell has been genetically modified in one or more genes identified herein so that the function of the modified gene(s) in a target cell differentiated from the genetically modified source cell is inhibited.

In some embodiments, a source cell has also been genetically modified to comprise a nucleic acid encoding a CAR (i.e., a chimeric antigen receptor). Nucleic acids encoding CARs can be introduced into a source cell by methods known in the art, such as 7-retroviral or lentiviral transduction, and CRISPR-Cas9, TALEN or ZFN mediated gene editing (Themeli et al., Generation of tumor-targeted human T lymphocytes from induced pluripotent stem cells for cancer therapy, Nat Biotechnol, 2013, 31(10): 928-33; Sadelain et al., Therapeutic T cell engineering, 2017, Nature, 545: 423-431; Eyquem et al., Targeting a CAR to the TRAC locus with CRISPR/Cas9 enhances tumour rejection, 2017, Nature, 543: 113-17).

iPSC

iPSCs are usually generated directly from somatic cells. iPSCs can be induced in principle from any nucleated cell including, for example, mononucleocytes from blood and skin cells. In some embodiments, iPSCs may be generated from fully differentiated T cells; or from precursor T cells, such as thymocytes, which precursor T cells have begun or even completed the re-arrangement of their TCRs and exhibit an antigen specificity of interest. In another embodiment, an iPSC is transfected with one or more nucleic acid molecules coding for a TCR (such as rearranged TCR genes) directed to an antigenic determinant of interest (e.g., a tumor antigenic determinant). In one embodiment, an iPSC is derived from a cell which expresses a rearranged TCR, preferably a rearranged αβ TCR. In another embodiment, said cell expresses a rearranged γδ TCR. Examples of cells suitable for use in generating the iPSCs of the present invention include, but are not limited to CD4+ T cells, CD8+ T cells, NKT cells, thymocytes or other form of precursor T cells.

Methods for generating iPSCs from mature or differentiated cells (such as T cells or precursor T cells) are known to the person of skill in the art. For example, iPSCs can be derived by introducing a specific set of pluripotency-associated genes, or “reprogramming factors”, into a somatic cell type. A commonly used set of reprogramming factors (also known as the Yamanaka factors) are the genes Oct4 (Pou5f1), Sox2, c-Myc, and Klf4. While this combination may be the most conventional combination used for producing iPSCs, each of the factors can be functionally replaced by related transcription factors, miRNAs, small molecules, or even non-related genes such as lineage specifiers. For example, the induction of iPSCs following transfection of Oct 3/4, Sox2, Klf4 and c-Myc using a retroviral system has been achieved, as it has also been via the transfection of Oct4, Sox2, Nanog and Lin28 using a lentiviral system. The former set of transcription factors are known as the Yamanaka factors while the latter are commonly known as the Thomson factors. As would be appreciated by the person of skill in the art, a wide range of modifications to the basic reprogramming factor expression vectors have been made and new modes of delivery have been designed in order to increase efficiency and minimize or remove vector sequences that might otherwise be integrated into the reprogrammed iPSC genome. These methods are well known to the skilled person and include, but are not limited to: single cassette reprogramming vectors with Cre-Lox mediated transgene excision, and reprogramming by non-integrating viruses such as adenovirus or Sendai virus. Alternatively, expression of reprogramming factors as proteins provides a means of generating iPSCs which have not undergone integration of the introduced vector DNA into the germline. Non-viral reprograming methods have also been developed. These include, but are not limited to: mRNA Transfection (Warren et al (2010)), miRNA Infection/Transfection Subramanyam et al (2011); PiggyBac (Kaji et al (2009); Woltjen et al (2009)); Minicircle Vectors (Narsinh et al (2011)); and Episomal Plasmids (Chuo et al (2011)). Various small molecules have been shown in the art to enhance reprogramming efficiency; see, e.g., those listed in the following table.

Compounds increasing iPSC reprogramming efficiency

| Treatment | Process affected |
|---|---|
| Valproic acid | Histone deacetylase inhibition |
| Sodium butyrate | Histone deacetylase inhibition |
| PD0325901 | MEK inhibition |
| A-83-01 | TGFβ-inhibition |
| SB43152 | TGFβ-inhibition |
| Vitamin C | Enhances epigenetic modifiers, promotes |
| | survival of antioxidant effects |
| Thiazovivin | ROCK inhibitor, promotes cell survival |
| PS48 | P13K/Akit activation, promotes glycolysis |
| 5% Oxygen | Promotes glycolysis |

In some embodiments, a source cell is an induced pluripotent stem cell (iPSC). In some embodiments the iPSCs are derived by episomal or Sendai viral transformation.

In some embodiments the iPSCs are >98% positive for SSEA-4 and TRA-1-60.

In some embodiments, the subject source cell is a cell that is more differentiated towards an immune cell as compared to a pluripotent stem cell.

In some embodiments, stem cells can be genetically modified using a non-viral method. In some embodiments, iPSCs can be modified using a non-viral method, e.g., CRISPR/Cas editing system.

In some embodiments, Chimeric Antigen Receptors (CARs) can be introduced into iPSC using a non-viral editing system, e.g., CRISPR-Cas9.

In some embodiments, the Knocking-Out (KO) of genetic material can be manipulated in iPSCs using a non-viral editing system, e.g., CRISPR-Cas9. In some embodiments, the KO of either DGKα and/or DGKζ genes in iPSCs is achieved using a non-viral editing system, e.g., CRISPR-Cas9. In some embodiments, iPSCs comprise a double KO of DGKαζ genes, for which a genetic manipulation is carried out using a non-viral editing system, e.g., CRISPR-Cas9.

In some embodiments, iPSC source cells are genetically modified to inhibit the expression of the DGKαζ genes (DGKαζ KO iPSC).

In some embodiments, iPSC source cells are genetically modified to express a chimeric antigen receptor (CAR), wherein said receptor comprises an antigen recognition moiety directed to an antigenic determinant, wherein said antigen determinant is of the tumor-associated antigen TAG-72 (TAG-72 CAR iPSC).

In some embodiments, iPSC source cells are genetically modified to inhibit the expression of the DGKαζ genes and further express a TAG-72 CAR (TAG-72 CAR/DGK KO iPSCs). In some embodiments, such genetically modified iPSCs maintain pluripotency and the ability to be propagated in culture.

In some embodiments, iPSC source cells can differentiate into CD34+ cells. In some embodiments, both the DGK KO iPSC and/or the TAG-72 CAR/DGK KO iPSCs can differentiate into CD34+ hemogenic progenitors.

In some embodiments, iPSC source cells can differentiate into CD45+ CD56+ NK cells. In some embodiments, DGK KO iPSCs and/or the TAG-72 CAR/DGK KO iPSCs can differentiate into mature NK cells, expressing normal NK stimulatory receptors.

In some embodiments, iPSC source cells can differentiate into progenitor T cells. In some embodiments, both the DGK KO iPSC and/or the TAG-72 CAR/DGK KO iPSCs can differentiate into mature CD3+ T cells, with predominately CD8+ phenotype. In some embodiments, both the DGK KO iPSC and/or the TAG-72 CAR/DGK KO iPSCs can differentiate into mature CD3+ T cells that express either TCR αβ or TCRγδ.

In some embodiments, CARs are introduced into iPSC using a non-viral editing system, e.g., CRISPR-Cas9. In some embodiments, the resulting genetically edited iPSC can differentiate into CD34+ cells and/or progenitor T cells.

In some embodiments, iPSC-derived CD34+ cells are able to differentiate into NK cells (iNK cells). In some embodiments, both the DGK KO iPSC-derived CD34+ cells and/or the TAG-72 CAR/DGK KO iPSCs-derived CD34+ cells, can differentiate into NK cells with no significant impact on the efficiency of the differentiation process.

In some embodiments, either the single DGKα/DGKζ or double DGKαζ KO in iPSC and/or TAG-72 CAR iPSC, does not affect the normal phenotype of said iPSC-derived NK cells and/or TAG-72 CAR iPSC-derived NK cells following differentiation.

In some embodiments, either the single DGKα/DGKζ or double DGKαζ KO in iPSC and/or TAG-72 CAR iPSC, does not affect the normal phenotype of said iPSC-derived T cells and/or TAG-72 CAR iPSC-derived T cells following differentiation.

### Mesoderm Cells

Three major cell populations (primary germinal layers) appear during embryonic development: mesoderm, ectoderm and endoderm. These cell populations are formed through a process known as gastrulation and following this process each primary germ cell layer generates a specific set of cell populations and tissues. For example, mesendoderm or mesoderm population gives rise to the heart, blood vessels and blood cells (e.g. immune cells).

To form immune cells, mesoderm cells are induced into multiple differentiation stages, each represented by a subpopulation of cells with distinct potential/phenotype that can be largely grouped into: hemogenic endothelium (HE), hematopoietic stem cells (HSCs), progenitors and immune cells. Each differentiation stages moves the cell closer to its final cell type by, for example, changing its gene expression that limits its potential to become a different cell type.

In some embodiments, the subject source cell is a mesoderm cell capable of differentiating into an immune cell.

### Hematopoietic Lineage Cells

The term “hematopoietic lineage cell” should be understood as any cell differentiated from a mesoderm cell (which can be obtained from pluripotent cells), and includes, for example, HE, pre-HSC and HSCs and progenitors (e.g. multipotent progenitor cells, lymphoid progenitor cells such as common lymphoid progenitor cells, early thymic progenitor cells, pre-T cell progenitor cells, pre-NK progenitor cells, T progenitor cells, NK progenitor cells, and myeloid progenitor cells such as common myeloid progenitor cells). The term “HE/HSC” is used here to refers to a subclass of hematopoietic lineage cells. HE/HSC are CD34+ stem cells capable of giving rise to both myeloid (e.g. macrophages and monocytes) and lymphoid cell types (e.g. B cells, T cells or NK cells), and include HE, pre-HSC and HSC. iCD34+ cells represent CD34+ expressing cells which have been differentiated from iPSC.

During embryogenesis, multipotent mesodermal cells differentiate into an endothelial cell lineage. HE is a subset of this lineage which acquires hematopoietic potential and gives rise to multilineage hematopoietic stem and progenitor cells in a process called endothelial-to-hematopoietic transition. The process that leads to hemogenic specification of endothelial cells and to the generation of HSC from hemogenic endothelium is still under discussion.

HSCs are blood stem cells that theoretically have the ability to become any blood cell of the lymphoid and myeloid lineages through the process of haematopoiesis. HSCs can be found in adult bone marrow, peripheral blood, and umbilical cord blood. HSCs can be collected from bone marrow, peripheral blood, and umbilical cord blood by established techniques, and are commonly associated with CD34+ expression.

In some embodiments, human HSCs can be defined as being CD34+ CD38− CD90+ CD45RA−. In some embodiments human HSCs can be defined as CD34+CD43+CD45+. In some embodiments human HSCs can be defined as CD34+CD133+.

When an HSC is derived from a pluripotent stem cell, it is often referred to as iHSC (induced haematopoietic stem cell).

A “pre-HSC” is to be understood as a cell differentiated from an HE cell, but which does not express the typical HSC markers, for example is a CD45− cell that expresses CD34.

In some embodiments a cell cultured from a pluripotent stem cell (such as an iPSC), which has undergone some differentiation in the culture towards an immune cell, but has not fully differentiated into an immune cell is used as a source cell.

In some embodiments an hematopoietic lineage cell capable of differentiating into an immune cell is used as a source cell. In some embodiments the hematopoietic lineage cell has not yet been differentiated into hematopoietic stem cells (HSCs), e.g. is a hemogenic endothelium (HE) or a pre-HSC. In some embodiments this hematopoietic lineage cell is an HSC.

In some embodiments the subject source cell is a myeloid progenitor cell, e.g., a common myeloid progenitor cell.

In some embodiments, the subject source cell is a lymphoid progenitor cell, e.g. multipotent progenitor cells, common lymphoid progenitor cells, early thymic progenitor cells, pre-T cell progenitor cells, pre-NK progenitor cells, T progenitor cells, NK progenitor cells (Galen et al., The unfolded protein response governs integrity of the haemopoietic stem cellpool during stress, Nature, 2014, Vol. 510(7504), p. 268). In some embodiments, the subject source cell is an immature T cell, such as a thymocyte, or an immature NK cell.

### Derived Cells

Stem cell-derived immune cells generated by the methods disclosed herein include hematopoietic lineage cells capable of differentiating into an immune cell, and immune cells. Examples of stem cell-derived immune cells are HE, pre-HSC, HSC, multipotent progenitor cells, lymphoid progenitor such as common lymphoid progenitor cells, early thymic progenitor cells, pre-T cell progenitor cells, pre-NK progenitor cells, T progenitor cells, NK progenitor cells, myeloid progenitor cells such as common myeloid progenitor cells, and immune cells.

An “immune cell”, as used herein, should be understood to include a cell of the mammalian immune system, for example, lymphocytes (T cells, B cells and NK cells), neutrophils, and monocytes (including macrophages and dendritic cells), and a cell line derived from cells of the mammalian immune system.

This disclosure is directed to providing stem cell-derived immune cell produced by differentiation having enhanced function. By “enhanced function”, it is meant that an immune cell provided as a result of modification or manipulation disclosed herein, displays an enhanced activity (e.g., cytotoxicity), proliferation, survival, persistence, resistance to immunosuppressive effects (e.g. TGFβ inhibition) and/or infiltration, as compared to a control immune cell (i.e., an immune cell without the modification or manipulation). Cytotoxicity of an immune cell refers to the ability of an immune cell to kill a target cell, generally through a receptor-based mechanism.

In some embodiments, the stem cell-derived immune cell in accordance with the present methods can be differentiated from a stem cell or other more differentiated cell (such as a cell cultured and differentiated from a stem cell).

In some embodiments, a stem cell-derived immune cell is a T cell. In some embodiments, the T cell is a NKT cell. In some embodiments, a stem cell-derived immune cell is a NK cell. In other embodiments a stem cell-derived immune cell is a macrophage or a macrophage lineage cell (e.g. monocytes, dendritic cell).

### T Cell

Reference to a “T cell” should be understood as a reference to any cell comprising a T cell receptor. In this regard, the T cell receptor may comprise any one or more of the α, β, γ or δ chains. As would be understood by the person of skill in the art, NKT cells also express a T cell receptor and therefore target antigen specific NKT cells can also be generated according to the present invention (and understood to be included in the definition of a T cell). The present invention is not intended to be limited to any particular sub-class of T cell, although in one embodiment the subject T cell expresses an α/β TCR dimer. In some embodiments, said T cell is a CD4+ helper T cell, a CD8+ killer T cell, or a NKT cell. Without limiting the present invention to any one theory or mode of action, CD8+ T cells are also known as cytotoxic cells. As a major part of the adaptive immune system, CD8+ T cells scan the intracellular environment in order to target and destroy, primarily, infected cells. Small peptide fragments, derived from intracellular content, are processed and transported to the cell surface where they are presented in the context of MHC class I molecules. However, beyond just responding to viral infections, CD8+ T cells also provide an additional level of immune surveillance by monitoring for and removing damaged or abnormal cells, including cancers. CD8+ T cell recognition of an MHC I presented peptide usually leads to either the release of cytotoxic granules or lymphokines or the activation of apoptotic pathways via the FAS/FASL interaction to destroy the subject cell. CD4+ T cells, on the other hand, generally recognise peptide presented by antigen presenting cells in the context of MHC class II, leading to the release of cytokines designed to regulate the B cell and/or CD8+ T cell immune responses. CD4+ T cells with cytotoxic activity have also been observed in various immune responses. Moreover, CD4+ CAR-T cells demonstrate equivalent cytotoxicity to CD8+ CAR-T cells in vitro, and even outperformed CD8+ CAR-T cells in vivo for longer anti-tumor activity (see, e.g., Wang et al., Glioblastoma-targeted CD4+ CAR T cells mediate superior antitumor activity, JCI Insight, 2018, 3(10):e99048; Yang et al., TCR engagement negatively affects CD8 but not CD4 CAR T cell expansion and leukemic clearance, Science Translation Medicine, 2017 November; 22; 9(417), eaag1209).

In some embodiments, a stem cell-derived immune cell is a cytotoxic immune cell, e.g., a cytotoxic lymphocyte.

In some embodiments, a stem cell-derived immune cell is a lymphoid lineage cell. In some embodiments a lymphoid lineage cell is a T cell. In some embodiments, the T cell is a CD4+ helper T cell, a CD8+ killer T cell, or an NKT cell.

In some embodiments, the stem-cell derived T cells disclosed herein demonstrate anti-tumor activity in vitro. In some embodiments, iPSC-derived TAG-72 CAR/DGKαζ KO T Cells show on-target activity against an ovarian cancer cell line in vitro. In some embodiments, TAG-72 CAR/DGKαζ KO T cells show enhanced killing of the human ovarian cancer cell line (OVCAR-3), compared to non-transfected iPSC-derived T cells and non-transfected T cells isolated from PBMC.

In some embodiments, the iPSC-derived T cells retain function in the immunosuppressive microenvironment of tumors. In some embodiments, iPSC-derived DGKαζ KO T cells have reduced sensitivity to the suppressive effect of tumors, as demonstrated, e.g., by such cells retaining in vitro cytotoxic function against OVCAR-3 cells in the presence of TGFβ, one of the key mediators of immunosuppression in the tumor microenvironment.

NK cell Natural killer T cells (also called NKT or T/NK cells) are a specialised population of T cells that express a semi-invariant T cell receptor (TCR α β) and surface antigens typically associated with natural killer cells. The TCR on NKT cells is unique in that it commonly recognizes glycolipid antigens presented by the MHC I-like molecule CD1d. Most NKT cells express an invariant TCR alpha chain and one of a small number of TCR beta chains. The TCRs present on type I NKT cells commonly recognise the antigen alpha-galactosylceramide (alpha-GalCer). Within this group, distinguishable subpopulations have been identified, including CD4⁺CD8⁻ cells, CD4⁻CD8⁺ cells and CD4⁻CD8⁻ cells. Type II NKT cells (or noninvariant NKT cells) express a wider range of TCR α chains and do not recognise the alpha-GalCer antigen. NKT cells produce cytokines with multiple, often opposing effects, for example either promoting inflammation or inducing immune suppression including tolerance. As a result, they can contribute to antibacterial and antiviral immune responses, promote tumor-related immunosurveillance, and inhibit or promote the development of autoimmune diseases, Like natural killer cells, NKT cells can also induce perforin-, Fas-, and TNF-related cytotoxicity. Accordingly, reference to T cells should be understood to include reference to NKT cells.

Natural killer (NK) cells are a type of cytotoxic lymphocyte that forms part of the innate immune system. NK cells provide rapid responses to virus-infected cells, acting at around 3 days after infection, and also respond to tumor formation. Typically, immune cells such as T cells detect major histocompatibility complex (MHC) presented on infected or transformed cell surfaces, triggering cytokine release and resulting in lysis or apoptosis of the target cell. NK cells, however, have the ability to recognize stressed cells in the absence of antibodies or MHC, allowing for a much faster immune reaction. This role is especially important because harmful cells that are missing, or have lower than normal levels, MHC I markers cannot be detected and destroyed by other immune cells, such as T cells. In contrast to NKT cells, NK cells do not express TCR or CD3 but they usually express the surface markers CD16 (FcγRIII) and CD56. In some embodiments, a stem cell-derived immune cell is a NK cell.

In some embodiments, disclosed herein are stem-cell derived NK cells having enhanced function. In some embodiments, iPSC-derived DGKαζ KO NK cells display an enhanced functionality and survival period compared to their respective non-transfected iPSC-derived NK control cells in vitro extended co-cultures with target cancer cells. The significance of the DGKαζ KO for the survival and enhancement cytotoxic anti-tumor function of the iPSC-derived NK cells has been established herein.

In some embodiments, iPSC-derived TAG-72 CAR/DGKαζ KO NK cells shows enhanced killing of tumor cells in vitro (compared to unedited iPSC-derived NK cells and NK cells isolated from the peripheral blood mononuclear cell (PBMC) fraction of healthy adult donors).

In some embodiments, disclosed herein are stem-cell derived NK cells with an improved proliferative capacity. In some embodiments, disclosed herein are iPSC-derived DGKαζ KO NK cells which demonstrate improved proliferative capacity compared to the non-transfected iPSC-derived NK cells.

In some embodiments, the iPSC-derived NK cells retain function in the immunosuppressive microenvironment of tumors. In some embodiments, disclosed herein are iPSC-derived DGKαζ KO NK cells with reduced sensitivity to the suppressive effect of tumors, as demonstrated, e.g., by such cells retaining in vitro cytotoxic function in the presence of TGFβ, one of the key mediators of immunosuppression in the tumor microenvironment.

In some additional embodiments, disclosed herein are iPSC-derived TAG-72/DGKαζ KO NK cells which retain function in the immunosuppressive microenvironment of tumors. In some embodiments, iPSC-derived TAG-72/DGKαζ KO NK cells disclosed herein are able to induce killing of tumor cells, e.g. OVCAR-3 cells line, under conditions representing immunosuppressive microenvironment. In some embodiments, said iPSC-derived TAG-72/DGKαζ KO NK cells retain in vitro cytotoxic function in the presence of TGFβ.

In some embodiments, stem-cell derived NK cells disclosed herein demonstrate anti-tumor activity in vivo. It is disclosed herein that treatment of tumor-bearing mice with iPSC-derived DGKαζ KO NK cells resulted in reduced tumor size and improvement in survival, with or without cytokine co-administration, compared to treatment with unedited (non-transfected) iPSC-derived NK cells.

In some embodiments, the stem-cell derived TAG-72 CAR/DGKαζ KO NK cells disclosed herein demonstrate prolonged anti-tumor activity in vivo. It is shown herein that treatment of tumor-bearing mice with iPSC-derived TAG-72 CAR/DGKαζ KO NK cells result in lower mean tumor burden and superior long-term efficacy, compared to either PBMC-NK cells, unedited (non-transfected) iPSC-derived NK cells, or the TAG-72 CAR iPSC-derived NK cells.

### Genes to Be Inhibited

In accordance with this disclosure, stem cell-derived immune cells with enhanced function can be generated by modifying source cells to inhibit the function of one or more genes identified herein and differentiating said modified source cell into a stem cell-derived immune cell.

By “inhibition of the function of a gene” as used herein, it is meant that the level and/or activity of the protein encoded by the gene is ultimately reduced or eliminated. Thus, the function of a gene can be inhibited as a result of manipulation or modification to the genomic DNA sequence of the gene (e.g., leading to a disruption of the gene), as a result of inhibiting the mRNA (e.g., reducing the level or function of the mRNA, e.g., by inhibiting transcription or translation), or as a result of inhibiting the protein (e.g., by reducing the level or activity of the protein). In some embodiments, the extent of inhibition is at least 20%, 30%, 40%, 50%, 60%, 70%, 80%, 90%, 95% or more, when the level and/or activity of the protein encoded by a gene in a modified cell is compared to the level and/or activity of the protein in an unmodified cell.

In accordance with this disclosure the gene whose function is to be inhibited is selected from the group consisting of DGKα and DGKζ.

### DGKα and DGKζ

Diacylglycerol kinase (DGK) is an enzyme that phosphorylates diacylglycerol (DAG) to phosphatidic acid (PA). There are multiple isoforms, two of which, DGKα and DGKζ, have strong functional links to immune cells. In both T and NK cells, DAG is one of the major messengers of activation and function, and DGKα and DGKζ act as a novel class of immune “check points” which can reduce the DAG dependent activation (Riese et al., Diacylglycerol Kinases (DGKs): Novel Targets for Improving T Cell Activity in Cancer, Frontiers in cell and developmental biology, 2016, Vol. 4, pp. 108; Noessner, DGK-alpha: A Checkpoint in Cancer-Mediated Immuno-Inhibition and Target for Immunotherapy, Frontiers in cell and developmental biology, 2017, Vol. 5, pp. 16). DAG interacts with essential proteins involved in CD3 signaling such as protein kinase C (PKC) and Ras activating protein (RasGRP1). Thus, activation of DGK is believed to result in downregulation of TCR distal molecules, including extracellular signal-related kinases 1/2 (ERK1/2). DGKα and DGKζ are dominantly expressed in T and NK cells, and their functions do not appear to be fully redundant because their expression and activation are regulated in a disparate manner.

In accordance with this disclosure, stem cells wherein the function of at least one of DGKα and DGKζ genes has been inhibited are still capable of differentiation, and stem cell-derived immune cells, differentiated from the modified stem cells, comprise enhanced function.

### Inhibiting the Function of a Gene

Inhibition of the function of a gene can be achieved by a variety of approaches, for example, through gene editing, inhibiting translation via, for example, RNA interference or antisense oligonucleotides, or through the use of compounds such as small molecules or antibodies that directly antagonize the protein product.

### Inhibiting Through Gene Editing

In some embodiments, inhibition of the function of a gene is achieved through the use of a gene editing system that modifies the genomic sequence of a gene.

A gene editing system typically involves a DNA-binding protein or DNA-binding nucleic acid, coupled with a nuclease. The DNA-binding protein or DNA-binding nucleic acid specifically binds to or hybridizes to a targeted region of a gene, and the nuclease makes one or more double-stranded breaks and/or one or more single-stranded breaks in the targeted region of the gene. The targeted region can be the coding region of the gene, e.g. in an exon, near the N-terminal portion of the coding region (e.g., in the first or second exon). The double-stranded or single-stranded breaks may undergo repair via a cellular repair process, such as by non-homologous end-joining (NHEJ) or homology-directed repair (HDR). In some instances, the repair process introduces insertion, deletion, missense mutation, or frameshift mutation (including, e.g., biallelic frameshift mutation), leading to disruption of the gene and inhibition of the function of the gene.

Examples of gene editing systems include a fusion comprising a DNA-binding protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)/Cas system. However, the invention should not be limited to these types of gene editing systems. Rather, any type of inhibitor known in the art or to be identified can be used to inhibit the function of a gene.

### ZFPs and TALENs

In some embodiments, inhibiting of the function of a gene is achieved by utilizing a gene editing system that includes a DNA-binding protein such as one or more zinc finger proteins (ZFP) or a transcription activator-like protein (TAL), fused to an endonuclease. Examples include ZFNs, TALEs, and TALENs.

The DNA binding domains of ZFPs and TAL can be “engineered” to bind to a target DNA sequence of interest. For example, one or more amino acids of the recognition helix region of a naturally occurring zinc finger or TALE protein can be modified so as to direct binding to a predetermined DNA sequence. Criteria for rational design are described, e.g., U.S. Pat. Nos. 6,140,081, 6,453,242, 6,534,261, WO 98/53058, WO 98/53059, WO 98/53060, WO 02/016536, WO 03/016496, and U.S. Publication No. 20110301073 A1.

In some embodiments, the DNA-binding protein comprises a zinc-finger protein (ZFP) or one or more zinc finger domains of a ZFP. ZFP or domains thereof bind to DNA in a sequence-specific manner through one or more “zinc fingers” (regions of amino acids within the binding domain whose structure is stabilized through coordination of a zinc ion). Sequence-specificity of a natural occurring ZFP can be altered by making amino acid substitutions at certain positions on a zinc finger recognition helix. In addition, many engineered, gene-specific zinc fingers are available commercially (see, e.g., the CompoZr platform for zinc-finger construction, developed by Sangamo Biosciences (Richmond, Calif., USA) in partnership with Sigma-Aldrich (St. Louis, Mo., USA)). Thus, in some embodiments, the ZFP is engineered to bind to a target sequence within a gene which is identified herein to be inhibited. Typical target sequences include exons, regions near the N-terminal region of the coding sequence (e.g., first exon, second exon), and the 5′ regulatory region (promoter or enhancer regions). A ZFP is fused to an endonuclease or a DNA cleavage domain to form a zinc-finger nuclease (ZFN). Examples of DNA cleavage domains include a DNA cleavage domain of a Type IIS restriction enzyme.

In some embodiments, a ZFN is introduced into a cell (e.g., a stem cell) via transfection of a nucleic acid construct (e.g., a plasmid, mRNA or viral vector) comprising a nucleic acid sequence encoding the ZFN. The ZFN is then expressed in the cell from the construct and leads to editing and disruption of a target gene. In some embodiments, a ZFN is introduced into a cell in its protein form.

In some embodiments, the DNA-binding protein comprises a naturally occurring or engineered transcription activator-like protein (TAL) DNA binding domain, such as in a transcription activator-like protein effector (TALE) protein. See, e.g., US 20110301073 A1, incorporated herein by reference. A TALE DNA binding domain is a polypeptide comprising one or more TALE repeats, with each repeat being 33-35 amino acids in length and including 1 or 2 DNA-binding residues. It has been determined that an HD sequence at positions 12 and 13 of a TAL repeat leads to a binding to cytosine (C), NG binds to T, NI to A, and NN binds to G or A. See, US 20110301073 A1. In some embodiments, TALEs can be designed to have an array of TAL repeats with specificity to a target DNA sequence of interest within a gene identified herein to be inhibited. Custom-designed TALE arrays are also commercially available through Cellectis Bioresearch (Paris, France), Transposagen Biopharmaceuticals (Lexington, Ky., USA), and Life Technologies (Grand Island, N.Y., USA). In some embodiments, a TAL DNA binding domain is fused to an endonuclease to form a TALE-nuclease (TALEN), which cleaves a nucleic acid target sequence within a gene identified herein to be inhibited.

In some embodiments, a TALEN is introduced into a cell (e.g. a stem cell) via transfection of a nucleic acid construct (e.g., a plasmid, mRNA or viral vector) comprising a nucleic acid sequence encoding the TALEN. The TALEN is then expressed in the cell from the construct and leads to editing and disruption of a target gene. In some embodiments, a TALEN is introduced into a cell in its protein form.

### CRISPR Cas

In some embodiments, inhibition of the function of a gene is achieved by utilizing a CRISPR (for “Clustered Regularly Interspaced Short Palindromic Repeats”)/Cas (for “CRISPR-associated nuclease”) system for gene editing. CRISPR/Cas is well known in the art with reagents and protocols readily available (Mali et al., RNA-Guided Human Genome Engineering via Cas9, Science, Feb. 15, 2013, Vol. 339(6121), p. 823(4); Hsu et al., Development and applications of CRISPR-Cas9 for genome engineering, Cell, 5 Jun. 2014, Vol. 157(6), pp. 1262-1278; Jiang et al., RNA-guided editing of bacterial genomes using CRISPR-Cas systems, Nature Biotechnology, 2013, Vol. 31(3), p. 233; Anzalone et al., Search-and-replace genome editing without double-strand breaks or donor DNA, Nature, December 2019, Vol. 576(7785), pp. 149-157; Komor et al., Programmable editing of a target base in genomic DNA without double-stranded DNA cleavage, 2016, Nature 533: 420-424; Gaudelli et al., Programmable base editing of A•T to G•C in genomic DNA without DNA cleavage, Nature, 2017, Vol. 551(7681), p. 464). Exemplary CRISPR/Cas gene editing protocols are described in Jennifer Doudna, and Prashant Mali, CRISPR-Cas: A Laboratory Manual, 2016 (CSHL Press, ISBN: 978-1-621821-30-4) and Ran, et al., Genome engineering using the CRISPR-Cas9 system, Nature Protocols, 2013, Vol. 8(11), p. 2281.

A CRISPR/Cas system generally comprises two components: (1) an RNA-dependent DNA nuclease, also referred to herein as a CRISPR endonuclease or a Cas protein, such as Cas9, Cas12 or other alternative nucleases; and (2) a non-coding short “guide RNA” which comprises either a dual RNA comprising a crRNA (“CRISPR RNA”) and a tracrRNA (“transactivating crRNA”), or a single-chain full length guide RNA, and comprises a targeting sequence that directs the nuclease to a target site in the genome. The guide RNA (gRNA) directs the nuclease to the target site where the nuclease generates a double-stranded break (DSB) in the DNA at the target site. The resulting DSB is then repaired by one of two general repair pathways: the Non-Homologous End Joining (NHEJ) pathway and the Homology Directed Repair (HDR) pathway. The NHEJ repair pathway is the most active repair mechanism, capable of rapidly repairing DSBs, but frequently results in small nucleotide insertions or deletions (Indels) at the DSB site, resulting in a frameshift mutation which leads to production of a non-functional gene product. The HDR pathway is less efficient but with high-fidelity. When a CRISPR endonuclease is provided with a DNA template homologous to the break region, the double-stranded break is repaired using the homologous DNA template via HDR. The HDR pathway allows insertion of large gene inserts into cells along with RNPs.

Design or selection of a gRNA sequence that comprises a sequence targeting a target site in a gene of interest has been described in the art. The target site can include sequences of regulatory regions (such as promoters and enhancers), and sequences within the coding region (such as exons, e.g., exons near the 5′ end, or an exon encoding a particular domain or region of the protein). In some embodiments, a target site is selected based on its location immediately 5′ of a protospacer adjacent motif (PAM) sequence, such as typically NGG, or NAG.

A guide sequence is designed to include a targeting sequence having complementarity with a target sequence (a nucleotide sequence at a target site). Full complementarity is not necessarily required, as long as there is sufficient complementarity to cause hybridization between a guide sequence and a target sequence and promote formation of a CRISPR complex at the target site. In some embodiments, the degree of complementarity between the targeting sequence of a gRNA and a target sequence is at least 80%, 85%, 90%, 95%, 98%, 99% or higher (e.g., 100% or fully complementary).

In some embodiments, a guide sequence is at least 15 nucleotides, e.g., 15, 16, 17, 18, 19, 20, 21, 22, 23, 24, 25, 26, 27, 28, 29, 30, 35, 40, 45, 50, 55, 60, 65, 70 or 75 or more, nucleotides in length. In some embodiments, a guide sequence is not more than 75, 70, 65, 60, 55, 50, 45, 40, 35, 30, 25, or 20 nucleotides in length. In some embodiments, the targeting sequence portion of a guide sequence is about 20 nucleotides in length. Truncated gRNAs, with shorter regions (<20 nucleotides) of target complementarity, have been described as effective with improved target specificity (see, e.g., Fu et al., Improving CRISPR-Cas nuclease specificity using truncated guide RNAs, Nature Biotechnology, 2014, Vol. 32(3), p. 279). Thus, in some embodiments, the targeting sequence of a guide RNA is 17, 18, 19 or 20 nucleotides in length. In some embodiments, the targeting sequence of a guide RNA is fully complementary to a nucleotide sequence at a target site. In some embodiments where the targeting sequence of a guide RNA is not fully complementary to a nucleotide sequence at a target site, the portion of the targeting sequence that is close to the PAM sequence in the genome (also referred to as the seed region) is fully complementary to a nucleotide sequence at a target site. In other words, some variation in the nucleotides 5′ of the guide sequence (i.e., the non-seed region) is permissible. For example, a guide sequence can be designed to include a targeting portion of at least 17 nucleotides in length (e.g., 17, 18, 19 or 20 nucleotides in length), having a seed region of at least 17 nucleotides being fully complementary to at least 17 nucleotides in a target sequence.

Examples of target sequences in specific genes are provided in Table 1.

In some embodiments, a guide sequence includes a targeting sequence of 17-20 nucleotides, with at least the 17 nucleotides in the seed region (the 3′ portion of the targeting sequence) being fully complementary to at least 17 nucleotides in a target sequence, e.g., to the 17 nucleotides from the 3′ end of a target sequence.

TABLE 1

| Gene | | | |
|---|---|---|---|
| Official | Genomic | Target | Guide RNA Target Sequences (a nucleotide |
| Symbol | RefSeqGene | Transcript | sequence at a target site; 5′ to 3′) |
| DGKα | NC_000012.12 | ENSG00000065357 | TATCCTACAGATGATGCGAG (SEQ ID NO: 1) |
| | | | CTCTCAAGCTGAGTGGGTCC (SEQ ID NO: 2) |
| | | | CAGCACTAGCAGTGGCACGG (SEQ ID NO: 3) |
| | | | AGACCCAGCAGCACTAGCAG (SEQ ID NO: 4) |
| | | | GAGATTGACTATGATGGCAG (SEQ ID NO: 5) |
| | | | GCTCTGTCTCTCAAGCTGAG (SEQ ID NO: 6) |
| | | | CAGCCACTCGCATCATCTGT (SEQ ID NO: 7) |
| | | | GCTCAGACACATCCCAATCC (SEQ ID NO: 8) |
| DGKζ | NG_047092.1 | ENSG00000149091 | TAGGAAAGCCATCACCAAGT (SEQ ID NO: 9) |
| | | | ACGAGCACTCACCAGCATCC (SEQ ID NO: 10) |
| | | | CTAGGAGTCAGCGACATATG (SEQ ID NO: 11) |
| | | | GCAGAAGTCCCCGGACACGT (SEQ ID NO: 12) |
| | | | GTTCGAGACCAACGTGTCCG (SEQ ID NO: 13) |
| | | | CGGGGACTTCTGCTACGTTG (SEQ ID NO: 14) |
| | | | CCGCTCTGACTCGCTGCACG (SEQ ID NO: 15) |
| | | | CCCCGTGCAGCGAGTCAGAG (SEQ ID NO: 16) |

A gRNA database for CRISPR genome editing is publicly available, which provides exemplary sgRNA target sequences in constitutive exons of genes in the human genome or mouse genome (see, e.g., the gRNA-database provided by GenScript, and by Massachusetts Institute of Technology; see also, Sanjana et al., Improved vectors and genome-wide libraries for CRISPR screening, Nature Methods, 2014, Vol. 11(8), p. 783). In some embodiments, the gRNA sequence is or comprises a sequence with minimal off-target binding to a non-target gene.

Examples of Cas proteins or CRISPR endonucleases suitable for use herein include Cpf1 (Zetsche et al., Cpf1 Is a Single RNA-Guided Endonuclease of a Class 2 CRISPR-Cas System, Cell (Cambridge), 22 Oct. 2015, Vol. 163(3), pp. 759-771), Cas1, Cas1B, Cas2, Cas3, Cas4, Cas5, Cas6, Cas7, Cas8, Cas9 (also known as Csn1 and Csx12), Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, or Csf4, or a functional derivative thereof (i.e., a mutant form or a derivative of a naturally occurring CRISPR endonuclease, such as a fragment thereof, that substantially retains the RNA-dependent endonuclease activity of the naturally occurring form). See, e.g., US20180245091A1 and US20190247517A1. In some embodiments, a Cas protein is Cas9, e.g., Cas9 from S. pyogenes, S. aureus or S. pneumoniae. In some embodiments, the Cas protein is a Cas9 protein from S. pyogenes having the amino acid sequence provided in the SwissProt database under accession number Q99ZW2. A number of Cas proteins that have been identified post CRISPR-Cas9 provide desirable features. For example, CRISPR-Cas12 makes staggered cuts and can edit epigenomes—the chemical compounds that can tell genes to turn on or off. Cas13 influences gene expression by targeting RNA instead of DNA. CRISPR-CasX is smaller than Cas9 and can be used to control gene expression, not just to edit genes. CasY acts much like Cas9, but is made of a completely different protein structure, allowing it to function in different conditions.

In some embodiments, inhibition of the function of a gene is achieved through CRISPR-mediated gene editing, which comprises introducing into a cell (e.g., a pluripotent stem cell, or a iPSC, or a HE, or a HSC, or a progenitor cell) a first nucleic acid encoding a Cas nuclease, and a second nucleic acid encoding a guide RNA (gRNA) specific to a target sequence in a gene identified herein to be inhibited. The two nucleic acids can be included in one nucleic acid construct (or vector), or provided on different constructs (or vectors), to achieve expression of the Cas protein and the gRNA in the cell. Expression of the Cas nuclease and the gRNA in the cell directs the formation of a CRISPR complex at the target sequence, which leads to DNA cleavage.

In some embodiments, inhibition of the function of a gene is achieved through CRISPR-mediated gene editing, which comprises introducing into a cell a combination or complex between a gRNA and a Cas nuclease. In some embodiments, a Cas protein/gRNA combination or complex can be delivered into a cell via e.g., electroporation, particle gun, Calcium Phosphate transfection, cell compression or squeezing, liposomes, nanoparticles, microinjection, naked DNA plasmid transfer, protein transduction domain mediated transduction or virus mediated (including integrating viral vectors such as retrovirus and lentivirus, and non-integrating viral vectors such as adenovirus, AAV, HSV, vaccinia).

Regardless of the specific gene editing method used, in order to confirm that a gene sequence has been modified and the gene function has been inhibited, a variety of assays may be performed, including for example, by examining the DNA or mRNA via Southern and Northern blotting, PCR including RT-PCR, or nucleic acid sequencing, or by detecting the presence or activity of a particular protein or peptide via, e.g., immunological means (ELISAs and Western blot).

In some embodiments, the function of at least one of the DGKα and DGKζ genes is inhibited by introducing indel(s) into an early exon of at least one of these genes through a CRISPR/Cas9 system, which results in frame-shift mutation(s) in at least one of these gene such that no functional protein is translated from an edited gene. In some embodiments, the functions of the two genes are inhibited by introducing an indel into an early exon of the two genes using CRISPR/Cas9, resulting in a frame-shift mutation in the two genes such that no functional protein is translated from an edited gene. In some embodiments, the inhibition of one or of the two genes is done in combination with the inhibition of another gene.

CRISPR/Cas system can also be used without double-strand breaks or donor DNA, by using Nickases (i.e., CAS9 nickase) and High Fidelity Enzymes. See, e.g., Anzalone, A et al., Search-and-replace genome editing without double-strand breaks or donor DNA, Nature, December 2019, Vol. 576(7785), pp. 149-157; Komor et al., Programmable editing of a target base in genomic DNA without double-stranded DNA cleavage, Nature, 2016, 533: 420-424; Gaudelli et al., Programmable base editing of A•T to G•C in genomic DNA without DNA cleavage, Nature, 2017, Vol. 551(7681), p. 464).

Inhibiting Through Reducing or Eliminating the Level or Function of mRNA

In some embodiments, inhibition of the function of a gene is achieved by reducing or eliminating the level or function of the mRNA transcribed from the gene, i.e., inhibition of the mRNA. Unlike inhibition through a gene editing system, inhibition of mRNA is transient.

In some embodiments, inhibition of mRNA can be achieved through the use of e.g., an antisense nucleic acid, a ribozyme, a small interfering RNA (siRNA), a short hairpin RNA (shRNA), a miRNA (microRNA) or a precursor thereof, or a nucleic acid construct that can be transcribed in a cell to produce an antisense RNA, an siRNA, an shRNA, a miRNA or a precursor thereof.

Antisense—Antisense technology is a well-known method. An antisense RNA is an RNA molecule that is complementary to the full length or a part of an endogenous mRNA and blocks translation from the endogenous mRNA by forming a duplex with the endogenous mRNA. An antisense RNA can be made synthetically and introduced into a cell of interest (e.g., a stem cell), or made in the cell of interest through transcription from an exogenously introduced nucleic acid construct, to achieve inhibition of expression of a gene of interest. It is not necessary for an antisense RNA to be complementary to the full length mRNA from a gene of interest. However, an antisense RNA should be of a length sufficient for forming a duplex with the target mRNA and blocking translation based on the target mRNA. Typically, an antisense RNA is at least 15 nucleotides, e.g., 15, 16, 17, 18, 19, 20, 21, 22, 23, 24, 25, 26, 27, 30, 35, 40, 50, 75, 100, 200, 300, 400, 500 nucleotides or more in length. In some embodiments, an antisense RNA is not more than 500, 400, 300, 200, 100, 75 or 50 nucleotides in length.

Ribozyme—A ribozyme (i.e., catalytic RNA) can be designed to specifically pair with a target RNA and cleave the phosphodiester backbone at a specific location, thereby functionally inactivating the target RNA. See, e.g., U.S. Pat. Nos. 6,423,885, 5,254,678, and Perriman et al., Effective ribozyme delivery in plant cells, Proceedings of the National Academy of Sciences of the United States of America, Jun. 20, 1995, Vol. 92(13), pp. 6175-6179. A ribozyme can be made synthetically and introduced into a cell of interest (e.g., a stem cell), or made in the cell of interest through transcription from an exogenously introduced nucleic acid construct.

RNAi (RNA Interference)—Inhibition of gene expression or translation through RNAi is known in the art and can be achieved utilizing RNA molecules such as an siRNA (for “small interfering RNA”), shRNA (for “short hairpin RNA”), and a miRNA (for “microRNA”). siRNAs and shRNAs are known to be involved in the RNA interference pathway and interfere with the expression of a specific gene. siRNAs are small (typically 20-25 nucleotides in length), double-stranded RNAs and can be designed to include a sequence homologous to or complementary with a target mRNA (i.e., the mRNA transcribed from a gene of interest) or a portion of a target mRNA. shRNAs are cleaved by riobonuclease DICER to produce siRNAs. Given the sequence of a target gene, siRNAs or shRNAs can be designed and made either synthetically and introduced into a cell of interest (e.g., a stem cell), or made in a cell of interest (e.g., a stem cell) from an exogenously introduced nucleic acid construct encoding such an RNA. miRNAs are also small RNA molecules (generally about 21-22 nucleotides) that are processed from long precursors transcribed from non-protein-encoding genes, and interrupt translation through imprecise base-pairing with target mRNAs. miRNA or a precursor thereof (pri-miRNA or pre-miRNA) can be made synthetically and introduced to a cell of interest (e.g., a stem cell) or made in a cell of interest (e.g., a stem cell) from an exogenously introduced nucleic acid construct encoding either the miRNA or a precursor thereof.

In some embodiments, inhibition of mRNA can be achieved using a modified version of a CRISPR/Cas system where a Cas molecule that is an enzymatically inactive nuclease is used in combination with a gRNA targeting a gene of interest. The target site can be in the 5′ regulatory region (e.g., the promoter or enhancer region) of the gene. In some embodiments, the Cas molecule is an enzymatically inactive Cas9 molecule, which comprises a mutation, e.g., a point mutation, that eliminates or substantially reduces the DNA cleavage activity (see e.g., WO2015/161276). In some embodiments, an enzymatically inactive Cas9 molecule is fused, directly or indirectly, to a transcription repressor protein.

### Inhibiting Through Other Means

The invention incudes other methods known in the art for inhibiting the function of a gene, including for reducing the level or activity of the protein encoded by the gene, e.g. by introducing into a cell (e.g., a stem cell) a compound (e.g., a small molecule, an antibody, among others) that directly inhibits the activity of the protein encoded by the gene.

### CAR

In some embodiments, a cell (e.g. a stem cell) that has been modified to have inhibition of one or more selected genes has also been modified to contain a nucleic acid encoding a chimeric antigen receptor (or “CAR”).

In some embodiments, a nucleic acid encoding a CAR can be introduced into a cell prior to, simultaneous with, or subsequent to, the cell being modified to inhibit the function of a selected gene. In embodiments where the inhibition is transient (e.g., through an antisense RNA or RNAi), a nucleic acid encoding a CAR is preferably introduced into a cell prior to the cell being modified to achieve inhibition. In embodiments where the inhibition is permanent (e.g., through gene editing), a nucleic acid encoding a CAR can be introduced into a cell prior to, simultaneous with, or subsequent to, the cell being modified to achieve inhibition. In some embodiments, a nucleic acid encoding a CAR is designed to allow insertion by HDR at the target site of gene editing following the introduction of the DSBs, i.e., the gene is disrupted by knock-in or insertion of the CAR-encoding nucleic acid.

In some embodiments, a cell-derived (e.g. a hematopoietic lineage cell or an immune cells) from a cell (e.g. a stem cell) that has been modified to have inhibition of one or more selected genes, has also been modified to contain a nucleic acid encoding a chimeric antigen receptor (or “CAR”).

The term “chimeric antigen receptor” (“CAR”, also known as an “artificial T cell receptor”, “chimeric T cell receptor” and “chimeric immunoreceptors”) should be understood as a reference to engineered receptors which graft an antigen recognition moiety onto an immune cell. Generally speaking, a CAR is composed of an antigen recognition moiety specific for a target antigen, a transmembrane domain, and an intracellular/cytoplasmic signaling domain of a receptor natively expressed on an immune cell operably linked to each other. By “operably linked” is meant that the individual domains are linked to each other such that upon binding of the antigen recognition moiety to target antigen, a signal is induced via the intracellular signaling domain to activate the cell that expresses the CAR (e.g., a T cell or an NK cell) and enable its effector functions to be activated.

The antigen recognition moiety of CARs is an extracellular portion of the receptor which recognizes and binds to an epitope of a target antigen. The antigen recognition moiety is usually, but not limited to, an scFv.

The intracellular domain of a CAR can include a primary cytoplasmic signaling sequence of a naturally occurring receptor of an immune cell, and/or a secondary or costimulatory sequence of a naturally occurring receptor of an immune cell. Examples of primary cytoplasmic signaling sequences include those derived from TCR zeta, FcR gamma, FcR beta, CD3 gamma, CD3 delta, CD3 epsilon, CD5, CD22, CD79a, CD79b, and CD66d. In some embodiments, the intracellular signaling domain of a CAR comprises a cytoplasmic signaling sequence from CD3 zeta. In some embodiments, the intracellular signaling domain of a CAR can comprise a cytoplasmic signaling sequence from CD3 zeta in combination with a costimulatory signaling sequence of a costimulatory molecule. Examples of suitable costimulatory molecules include CD27, CD28, 4-1BB (CD137), OX40, CD30, CD40, PD-1, TIM3, ICOS, lymphocyte function-associated antigen-1 (LFA-1), CD2, CD7, LIGHT, NKG2C, B7-H3, and the like. In some embodiments, the cytoplasmic domain of a CAR is designed to comprise the signaling domain of CD3 zeta and the signaling domain of CD28.

The transmembrane domain of a CAR is generally a typical hydrophobic alpha helix that spans the membrane and may be derived from any membrane-bound or transmembrane protein. The transmembrane domain may be derived either from a natural or from a synthetic source. Where the source is natural, the domain may be derived from any membrane-bound or transmembrane protein. For example, transmembrane regions may be derived from the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine.

The terms “target antigen” should be understood as a reference to any proteinaceous or non-proteinaceous molecule expressed by a cell which is sought to be targeted by the receptor-expressing immune cells such as T cells or NK cells. A target antigen may be a “self” molecule (a molecule expressed in the body of a patient) or a non-self molecule (e.g., from an infectious microorganism). Target antigens referred to herein are not limited to molecules which are naturally able to elicit a T or B cell immune response; rather, a “target antigen” is a reference to any proteinaceous or non-proteinaceous molecule which is sought to be targeted. In some embodiments, a target antigen is expressed on the cell surface. It should be understood that a target antigen may be exclusively expressed by the target cell, or it may also be expressed by non-target cells. In some embodiments, a target antigen is a non-self molecule, or a molecule that is expressed exclusively by the cells sought to be targeted or expressed by the cells sought to be targeted at a significantly higher level than by normal cells. Non-limiting examples of target antigens include the following: differentiation antigens such as MART-1/MelanA (MART-I), gplOO (Pmel 17), tyrosinase, TRP-1, TRP-2 and tumor-specific multilineage antigens such as, MAGE-1, MAGE-3, BAGE, GAGE-1, GAGE-2, p15; overexpressed glycoproteins such as MUC1 and MUC16; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other tumor associated antigen include folate receptor alpha (FRα), EGFR, CD47, CD24, TSP-180, MAGE-4, MAGE-5, MAGE-6, RAGE, NY-ESO, pl 85erbB2, pl80erbB-3, cMet, nm-23H1, PSA, CA 19-9, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27. 29\BCAA, CA 195, CA 242, CA-50, CAM43, CD68\P1, CO-029, FGF-5, G250, Ga733\EpCAM, HTgp-175, M344, MA-50, MG7-Ag, NB/70K, NY-CO-1, RCAS 1, SDCCAG16, TA-90\Mac-2 binding protein\cyclophilin C-associated protein, TAAL6, TAG-72, TLP, TPS, PSMA, mesothelin, or BCMA.

In some embodiments, the target antigen is a tumor-associated antigen, in particular a protein, glycoprotein or a non-protein tumor-associated antigen.

In some embodiments, the target antigen is selected from the group consisting of CD47, folate receptor alpha (FRα) and BCMA

In some embodiments, the target antigen is a tumor-associated antigen, for example, the tumor-associated antigen TAG-72.

In other embodiments, the target antigen is a surface protein for example CD24, and in another embodiment a surface protein that can be used for tumor-targeting, for example, CD19 or CD20.

In some embodiments, the source cell comprises one or more nucleic acid molecule encoding a chimeric antigen receptor (“CAR”), wherein said receptor comprises an antigen recognition moiety directed to an antigenic determinant. In some embodiments the source cell expresses at least one CAR.

In other embodiments the derived cell comprises one or more nucleic acid molecule encoding a chimeric antigen receptor (“CAR”) wherein said receptor comprises an antigen recognition moiety directed to an antigenic determinant. In some embodiments the derived cell expresses at least one CAR.

It would be appreciated by the person of skill in the art that the mechanism by which genetic modifications (e.g. introduction of a nucleic acid molecule encoding a chimeric antigen receptor (“CAR”)) are introduced into the cell may take any suitable form which would be well known and understood by those of skill in the art. For example, genetic material is generally conveniently introduced to cells via the use of an expression construct.

CARs can be introduced into cell through transfection of plasmid DNA or mRNA; transduction of viral vectors including γ-retrovirus, lentivirus and adeno associated virus; and CRISPR-Cas9, TALEN or ZFN mediated gene editing.

### Methods of Differentiation

The present invention generally relates to methods and composition for modifying stem cells, in particular iPSCs, and further differentiating them into stem cell-derived immune cells that comprising enhanced activity.

Methods for differentiating a source cell (e.g. pluripotent stem cell) into immune cells (e.g. T cells or NK cells), are known in the art (Li et al., Human iPSC-Derived Natural Killer Cells Engineered with Chimeric Antigen Receptors Enhance Anti-tumor Activity, Cell Stem Cell, 2018, 23(2): 181-192 e5; Themeli et al., Generation of tumor-targeted human T lymphocytes from induced pluripotent stem cells for cancer therapy, Nat Biotechnol, 2013, 31(10): 928-33; Maeda et al., Regeneration of CD8alphabeta T Cells from T cell-Derived iPSC Imparts Potent Tumor Antigen-Specific Cytotoxicity, Cancer Res, 2016, 76(23): 6839-6850).

They typically follow three main phases: 1) generation and expansion of CD34+ cells from PSCs, 2) differentiation of progenitors/immune cells from CD34+ cells, and 3) expansion of progenitors/immune cells.

### Preparation of the Pluripotent Cells

In some embodiments the PSCs may be prepared and/or sorted prior differentiation towards mesoderm cells. The selection process may be based on one or more genes or one or more markers.

After reprogramming to generate iPSCs, cells can be characterised based on embryonic like morphology, transgene silencing after reprogramming, pluripotency assessment via alkaline phosphatase assay or detection of pluripotent and renewal markers such as TRA-1-60, TRA-1-81, Nanog and Oct4. Differentiation potential is monitored by embryoid body formation and/or teratoma formation. Karyotype analysis, identity matching and sterility are also usually assessed (Huang et al., Human iPSC banking: barriers and opportunities, Journal of Biomedical Science, Oct. 28, 2019, Vol. 26(1)).

A variety of methods are used for human iPSC maintenance and preparation these include feeder cell-dependent culture using inactivated murine embryonic fibroblast (MEF) cells, culturing of iPSC using MEF conditioned medium, creation of embryoid bodies from iPSCs and matrix dependent propagation using serum-free and feeder-free expansion media or MEF conditioned media. iPSCs are passaged using enzymatic or mechanical passaging methods, in general this is done when colonies become too large/dense or increased differentiation occurs. Optimal iPSC colonies are those observed to have defined edges and a uniform morphology across colonies.

Transcription Activator-Like Effector Nucleases (TALEN) and CRISPR/Cas9 are most commonly for genome editing of PSCs. Generally, a TALE array or CRISPR guide RNA is designed and cloned using cloning vectors. Single cell human PSCs are transfected or transduced with the vectors and targeted cells can be selected with FACS via a fluorescent reporter or by antibiotic section via a selection marker. After 1 to 2 weeks post enrichment, PSCs are picked and expanded for genomic DNA analysis and for targeted clone recovery and expansion. Both gene editing approaches have been used to generate knock-in and or knock-out iPSC lines (Hendricks et al., Genome Editing in Human Pluripotent Stem Cells: Approaches, Pitfalls, and Solutions, Cell stem cell, 7 Jan. 2016, Vol. 18(1), pp. 53-65).

In some embodiments the iPSCs are lifted from the adhesive state as single cells in solution using such as Accutase, then electroporated in the presence of RNP complexes containing the guide RNA for the particular gene KO, and/or plasmid containing a sequence which may be desired to knock into the cell at the site of the gene of interest.

In some embodiments, post gene-editing, iPSCs are left to stabilise and returned to normal culture prior to any further manipulation or testing in functional studies such as differentiation to NK cells.

Generation and Expansion of CD34+ Cells from Pluripotent Stem Cell

Most methods in the art begin by differentiating PSCs toward mesoderm cells. This is followed by differentiating mesodermal cells to HE/HSC which may also be expanded at the same time.

The PSC differentiation method may follow a variety of approaches which include embryoid body (EB) formation, feeder cell co-culture, two-dimensional extracellular matrix-coated culture and programming or reprograming using transcription factor transduction (Lim et al., Hematopoietic cell differentiation from embryonic and induced pluripotent stem cells Stem cell research & therapy, 18 Jun. 2013, Vol. 4(3), pp. 71; Tajer et al., Ex Vivo Expansion of Hematopoietic Stem Cells for Therapeutic Purposes: Lessons from Development and the Niche, Cells, 18 Feb. 2019, Vol. 8(2)). These methods can produce hematopoietic progenitors. Increasing evidence suggests that hemogenic endothelial (HE) cells are transient intermediates that contribute to de novo production of multipotent HSCs.

Three dimensional EBs of PSC differentiation mimic in vivo embryonic development thus several methods using EB formation have been developed for hematopoietic differentiation of PSCs. These include spontaneous EB formation, hanging-drop EB formation and spin-EB formation. To specifically induce a hematopoietic lineage, single-cell suspension of EBs are directed into methylcellulose culture medium that functions to support hematopoietic development in the presence of hematopoietic cytokines and growth factors. Spin EB method-based differentiation to HSCs may take from 4 to 11 days of culture.

Feeder co-culture is a method of culturing a layer of feeder cells together with PSCs to support them towards development of hematopoietic lineages in appropriate culture medium. Stromal cell co-culture is used to obtain HE/HSCs. OP9 stromal cells, stromal cells derived from the aorta-gonad-mesonephros region, fetal liver-derived stromal cells and bone marrow-derived stromal cells such as S17 and M210, as well as AFT04 stromal cells are example lines used for co-culture. Stromal cell co-culture methods are usually animal-derived and may be either serum dependent or independent.

Two-dimensional culture in dishes coated with extracellular matrices, such as collagen and fibronectin are used as monolayer cultures to differentiate PSCs. Matrices using human fibronectin or collagen IV are mainly utilised to generate hematopoietic progenitors. PSCs also differentiate into mesodermal cells in the presence of matrix components such as laminin, collagen I, entactin and heparin-sulfate proteoglycan as well as growth factors and several other undefined compounds. These mesodermal cells are able to induce hematopoietic cells after substitution with hematopoietic cocktail culture medium.

Several cell extrinsic factors are required in hematopoietic cocktail culture mediums for PSC differentiation to HE/HSCs. Combinations of chemicals, cytokines and growth factors include but are not limited to bFGF, BMP4, Activin A, SB431542, DKK, CHIR99021, VEGF, IL-6, IGF-1, IL-11, SCF, EPO, TPO, IL-3 and FLT3-L.

Transcription factor transduction via direct programming or reprogramming of endothelial cells to HE/HSC has been used for PSC to HSC differentiation. Transcription factors include but are not limited to ERG, HOXA5, HOXA9, HOXA10, LCOR, RUNX1, SPI1, FOSB, GFI1, ETV2 and GATA2.

Hematopoietic cocktails are used to expand CD34+ cells/HSCs. These include but are not limited to those mentioned above. Haematopoietic cocktail commercial kits are also available for the expansion of CD34+ cells.

Other small molecules, proteins and chemicals can also be used in conjunction with the above-mentioned cytokines/growth factors for HSC/CD34+ cell expansion. These include but are not limited to tetraethylenepentamine, HOXB4, prostaglandin E2, Stemreginin 1, UM729, UM171, Notch ligands, Angiopoietin-like 5, IGFBP2, pleiotrophin, valproic acid and small molecule inhibitors of histone deacetylase and DNA methyltransferase. Three-dimensional nanofiber scaffolds have also been used for HSC expansion.

Differentiation of HE/HSC into Lymphoid/Myeloid Lineage Cells

Besides bone marrow, a main source of HSC/CD34+ hematopoietic cell progenitors (HSCs) is the blood. Specifically, these may be isolated from peripheral blood or umbilical cord blood. iPSC cell lines are also a source of these cells. Enrichment of HSC/CD34+ hematopoietic progenitors can be carried out using anti-CD34 immunomagnetic particles/beads.

CD34+ progenitors can be differentiated and expanded with expansion cocktail that includes a variety of cytokines, growth factors and/or small molecules. Antibody staining and flow cytometric analysis of marker expression are used to monitor differentiation success. Haematopoietic potential is monitored by clonogenic colony forming unit assays. Relevant hematopoietic gene expression is monitored via PCR, transcriptome analysis or other molecular biology-based methods. Engraftment can be tested in animals such as NOD/SCID mice.

Cytokine and growth factor combinations are used to drive differentiation of HSCs to immune cells. For example, IL-3, SCF, IL-15 and FLT3-L are used in combination with IL-2, IL-15 and IL-7 for the generation of NK cells with EBs in the presence or absence of feeder cells such as EL081D2.

To induce lymphoid commitment, the OP9 cell line expressing Notch ligand Delta-like-1 (OP9-DLL1) or OP9-DLL4 has been utilised in the presence of SCF, FLT3L and IL-7 or IL-15 to generate NK cells without CD34 enrichment or spin EB formation.

Expansion of NK cells derived from PSCs/HSCs can be carried out using feeder cells such as K562 with membrane bound IL-5 and 4-1BB ligand. Other cell lines used for NK cells expansion include membrane bound IL-21 artificial antigen presenting cells. Commercial kits are available to differentiate HSCs to NK cells, e.g. StemSpan™ NK Cell Generation Kit (STEMCELL Technologies).

OP9 and OP9-DLL1 culture in the presence of FLT3L, TL-7 and TL-2 followed by subsequent stimulation with anti-CD3 anti-CD28 can also induce T cells. Commercial kits are also available to differentiate HSCs to T cells, e.g., StemSpan™ T Cell Generation Kit (STEMCELL Technologies).

Monocytes and macrophages can be generated using stromal cell-based methods using for example the SC17 cell line or spin EB methodology and subsequent culture in the presence of IL-3, CSF-1 and M-CSF.

### Pharmaceutical Composition and Therapeutic Use of the Modified Cells

In a further aspect, provided herein are compositions containing the cells produced by the methods disclosed herein, i.e., modified cells in which the function of one or more of the selected genes has been inhibited and cell derived from them.

In some embodiments, provided herein is a pharmaceutical composition containing cells produced herein, and a pharmaceutically acceptable carrier. A pharmaceutically acceptable carrier includes solvents, dispersion media, isotonic agents and the like. Examples of carriers include oils, water, saline solutions, gel, lipids, liposomes, resins, porous matrices, preservatives and the like, or combinations thereof. In some embodiments, the pharmaceutical composition is prepared and formulated for administration to patients, such as for adoptive cell therapy, typically in a unit dosage injectable form (solution, suspension, emulsion). In some embodiments, a pharmaceutical composition can employ time-released, delayed release, and sustained release delivery systems.

In some embodiments, a pharmaceutical composition comprises cells in an amount effective to treat or prevent a disease or condition, such as a therapeutically effective or prophylactically effective amount. In some embodiments, a pharmaceutical composition includes modified cells disclosed herein, in an amount of about 1 million to about 100 billion cells, for example, at least 1, 5, 10, 25, 50, 100, 200, 300, 400 or 500 million cells, up to about 1, 5, 10, 20, 30, 40, 50, 60, 70, 80, 90 or 100 billion cells.

In some embodiments, a pharmaceutical composition further comprises another active agent or drug, such as a chemotherapeutic agent.

In another aspect, provided herein are methods and uses of the modified cells disclosed herein, such as therapeutic methods and uses in adoptive cell therapy.

In some embodiments, a method includes administration of the modified cells disclosed herein or a composition comprising the modified cells disclosed herein to a subject having a disease or condition or at risk of developing the disease or condition.

In some embodiments, the disease or condition is a neoplastic condition (i.e., cancer), or a microorganism or parasite infection (such as HIV, STD, HCV, HBV, CMV, or antibiotic resistant bacteria), or an autoimmune disease (e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma).

In some embodiments, a neoplastic condition includes central nervous system tumors, retinoblastoma, neuroblastoma, paediatric tumors, head and neck cancers (e.g. squamous cell cancers), breast and prostate cancers, lung cancer (both small and non-small cell lung cancer), kidney cancers (e.g. renal cell adenocarcinoma), esophagogastric cancers, hepatocellular carcinoma, pancreaticobiliary neoplasias (e.g. adenocarcinomas and islet cell tumors), colorectal cancer, cervical and anal cancers, uterine and other reproductive tract cancers, urinary tract cancers (e.g. of ureter and bladder), germ cell tumors (e.g. testicular germ cell tumors or ovarian germ cell tumors), ovarian cancer (e.g. ovarian epithelial cancers), carcinomas of unknown primary, human immunodeficiency associated malignancies (e.g. Kaposi's sarcoma), lymphomas, leukemias, malignant melanomas, sarcomas, endocrine tumors (e.g. of thyroid gland), mesothelioma and other pleural or peritoneal tumors, neuroendocrine tumors and carcinoid tumors.

In some embodiments, the present method leads to treatment of the condition, i.e., a reduction or amelioration of the condition, or any one or more symptoms of the condition, e.g., by inhibiting tumor growth and/or metastasis in the context of treating a cancer, or by reducing the viral load and/or spread in the context of treating a viral infection. The term “treatment” does not necessarily imply a total recovery. In some embodiments, the present method leads to prophylaxis of a condition, i.e., preventing, reducing the risk of developing, or delaying the onset of the condition. Similarly, “prophylaxis” does not necessarily mean that a subject will not eventually contract the condition.

In some embodiments, the subject, e.g., patient, to whom the cells or compositions are administered is a mammal, typically a primate, such as a human.

In some embodiments, the cells or a composition comprising the cells are administered parenterally. The term “parenteral,” as used herein, includes intravenous, intramuscular, subcutaneous, and intraperitoneal administration.

The desired dosage of the derived cells or a composition comprising the derived cells can be delivered by a single administration, by multiple administrations, or by continuous infusion administration of the composition. Therapeutic or prophylactic efficacy can be monitored by periodic assessment of a treated subject.

In some embodiments, source cells are generated from cells isolated from a subject, then modified in accordance with the methods disclosed herein (inhibit the function of one or more genes), further differentiated and then administered to the same subject.

In some embodiments, adoptive cell therapy is carried out by allogeneic transfer, in which the source cells are generated from cells from a donor subject different from a subject who is to receive the cell therapy (recipient subject). In some embodiments, the donor and recipient subjects express the same HLA class or supertype.

### EXAMPLES

In the following examples, it has been illustrated, without limitation, that to generate stem cell-derived immune cells with enhanced function for tumor treatment, CRISPR/Cas9 gene editing technology was employed to eliminate the two DGK genes. Gene editing efficiency was examined by genomic DNA sequencing-based quantification. The iPSCs were lifted as single cells using accutase and then a Cas9 nuclease complex with specifically designed guide RNA was transfected into these iPSCs to ablate the immune regulator gene(s). The iPSCs were then differentiated towards CD34+ cells. The CD34+ cells were further differentiated towards iNK cells. The cytotoxicity was monitored in vitro.

### Example 1—Generation of TAG-72 CAR iPSC Single-Cell Clones

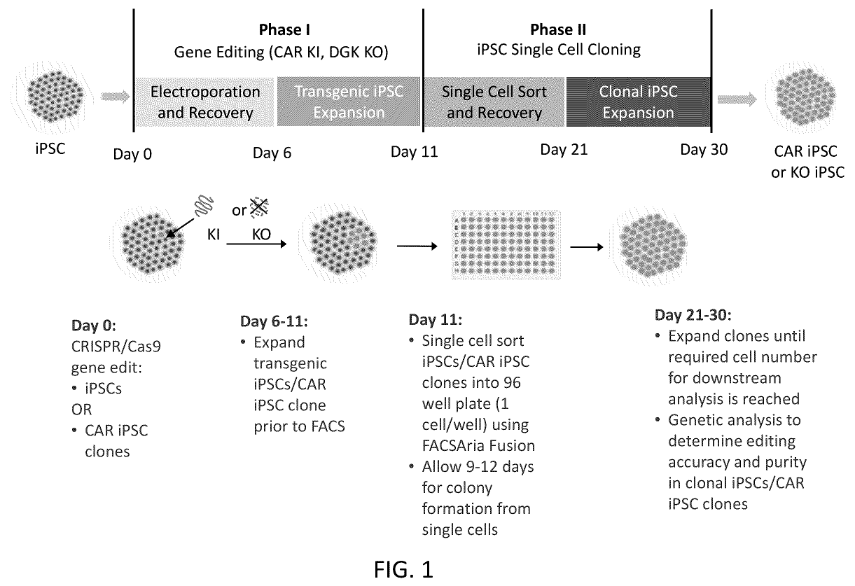

Stem cells like iPSCs can unlimitedly self-renew and differentiate into various cell types including hematopoietic stem cells (HSCs) and immune cells. Immune cells like T cells and NK cells have already been generated from iPSCs for cancer therapy (Li et al., Human iPSC-Derived Natural Killer Cells Engineered with Chimeric Antigen Receptors Enhance Anti-tumor Activity, Cell Stem Cell, 2018, 23(2): 181-192 e5; Themeli et al., Generation of tumor-targeted human T lymphocytes from induced pluripotent stem cells for cancer therapy, Nat Biotechnol, 2013, 31(10): 928-33; Maeda et al., Regeneration of CD8alphabeta T Cells from T cell-Derived iPSC Imparts Potent Tumor Antigen-Specific Cytotoxicity, Cancer Res, 2016, 76(23): 6839-6850). CAR-T or CAR-NK cells can be derived from lentiviral CAR transduced iPSCs following similar methods. To generate TAG-72 CAR iPSCs, TAG-72 CAR expression cassettes were introduced into the AAVS1 safe harbor site via the non-viral CRISPR/Cas9 gene knock-in method as described in WO2017/088012 and PCT/AU2020/050800 incorporated herein by reference. TAG-72 CAR positive iPSCs were sorted to generate purified TAG-72 CAR iPSC single-cell clones (FIG. 1). The CAR expression on the surface of the iPSC was characterised by FLOW cytometry.

### Single-Cell Cloning

For single cell cloning, firstly a 96-well plate was coated with CellAdhere™ Laminin-521 in PBS for 2 hrs at 37° C. The TAG-72 CAR iPSCs were sorted and seeded into the 96-well plates at a mean density of 1 cell/well. Transfected iPSCs were lifted as single cells using Accutase® and subsequently counted, washed and resuspended for staining with relevant antibody cocktails if required. Cells were stained at 4° C. for 15 minutes prior to sorting with the BD FACSAria™ Fusion. Propidium iodide (PI) staining was used for dead cell exclusion. Cells were gated to remove debris, doublets and dead cells, and were subsequently sorted into a 96-well plate. The plate was placed immediately into a 37° C. cell incubator for 48 hrs and daily media changes were performed for when cells are suitable for passaging.

### Example 2—Generation of DGKζ KO iPSCs (Pre-Sorted and Single-Cell Clones)

DGKα and DGKζ both have multiple transcripts. To ablate all of the major isoforms of DGKα and DGKζ, a list of guided RNAs were designed to target the DGKα gene and DGKζ gene (Table 1). Therefore, out-of-frame indels could be introduced into the early exons of their open reading frames (ORF) to disrupt the translation of DGKα and DGKζ.

To generate CRISPR DGKα and/or DGKζ gene double knock-out iPSCs, RNP complexes formed by representative gRNAs (SEQ ID NO: 3; SEQ ID NO: 11) were transfected into iPSCs using the Lonza 4D-Nucleofector™ system. Firstly, a 12 well plate was coated with CellAdhere™ Laminin-521 (STEMCELL Technologies) in PBS and incubated for 2 hrs at 37° C. iPSCs were pre-incubated with mTeSR Plus™ media (STEMCELL Technologies) containing RevitaCell™ Supplement (Life Technologies) for 2 hrs prior to transfection. RNPs were prepared by combining full length guide RNAs (gRNAs) with Lonza P3 buffer and Cas9. The RNP mixture was then incubated at room temperature for 10-20 minutes. After pre-incubation, iPSCs were lifted as single cells using Accutase® (Life Technologies) and 1×10⁶ cells per reaction were obtained for electroporation. To generate gene knock-out iPSCs, cells in Lonza P3 buffer and the RNP mixture were combined into PCR tubes; to generate gene knock-in iPSCs, cells in Lonza P3 buffer were combined with RNP mixture and donor DNA. The cells with RNP mixture were loaded into the Lonza 4D-Nucleofector™ for electroporation. Following this, mTeSR Plus™ with CloneR™ medium was added to the reaction and incubated at room temperature for 10 minutes. After incubation, cells were added to the Laminin-521 pre-coated plate in mTeSR Plus™ with CloneR™ medium. Daily media changes with mTeSR Plus™ were performed for 72 hrs and cells were passaged upon reaching ˜80% confluency (6-7 days post-electroporation). At the end of this phase the cells are termed DGK KO pre-sorted iPSCs.

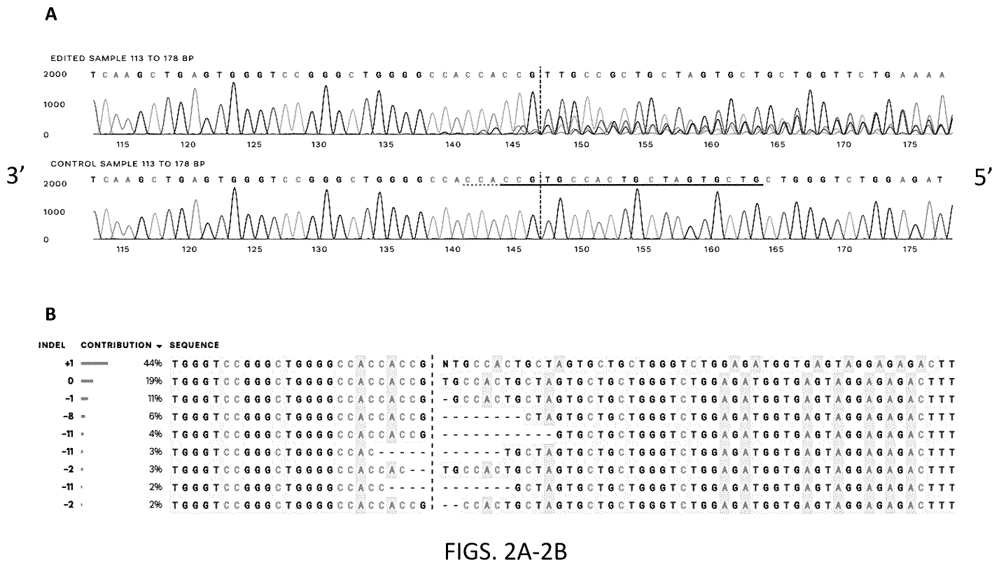

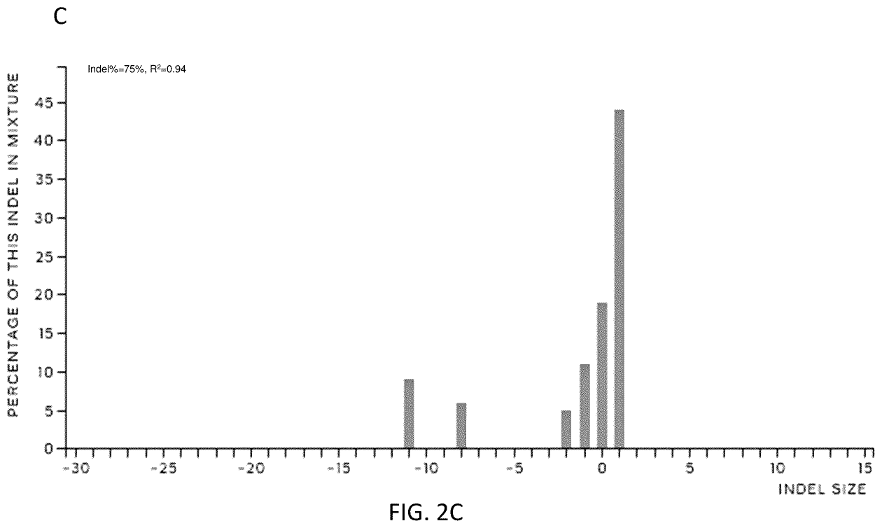

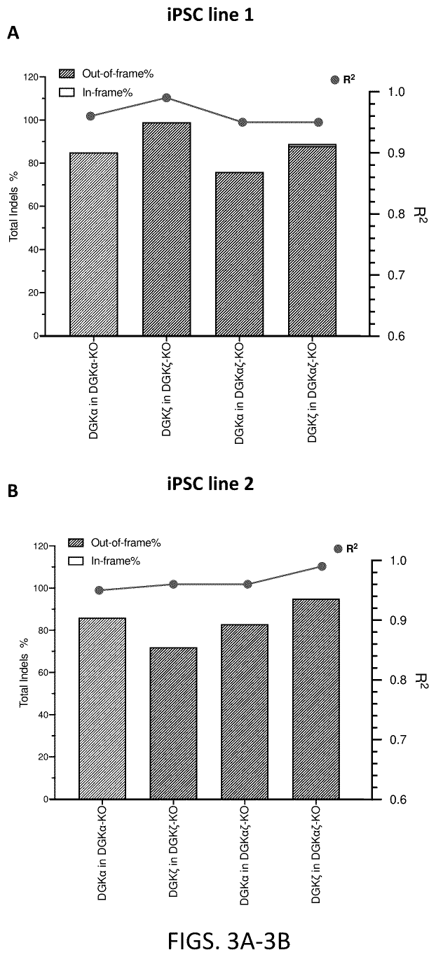

After RNP transfection and expansion, the genomic DNA was extracted from iPSCs for quantitative analysis of gene editing. The gene editing efficiency was analysed using the ICE (Inference of CRISPR Edits) assay (Hsiau et al., Inference of CRISPR Edits from Sanger Trace Data. bioRxiv, 2018, 10.1101/251082 (251082). DGKα gene editing efficiency analysis from a DGKα gRNA (SEQ ID NO: 3) iPSCs transfected sample was shown here as a representative result of ICE analysis (FIGS. 2A-2C). In addition, it is also shown that DGKα gRNA (SEQ ID NO: 3) and DGKζ gRNA (SEQ ID NO: 11) can introduce indels into multiple iPSCs lines (FIGS. 3A-3B).

To generate CRISPR DGKα and DGKζ gene single/double knock-out iPSCs and single-cell cloning two phases are required (FIG. 1). Firstly, phase I which is comprised of CRISPR/Cas9 gene editing including electroporation of iPSCs and their recovery, followed by the subsequent expansion of transgenic iPSCs. Phase II is comprised of single-cell cloning of transfected iPSCs which involves a single cell sort and subsequent clonal iPSCs expansion.

### Single-Cell Cloning

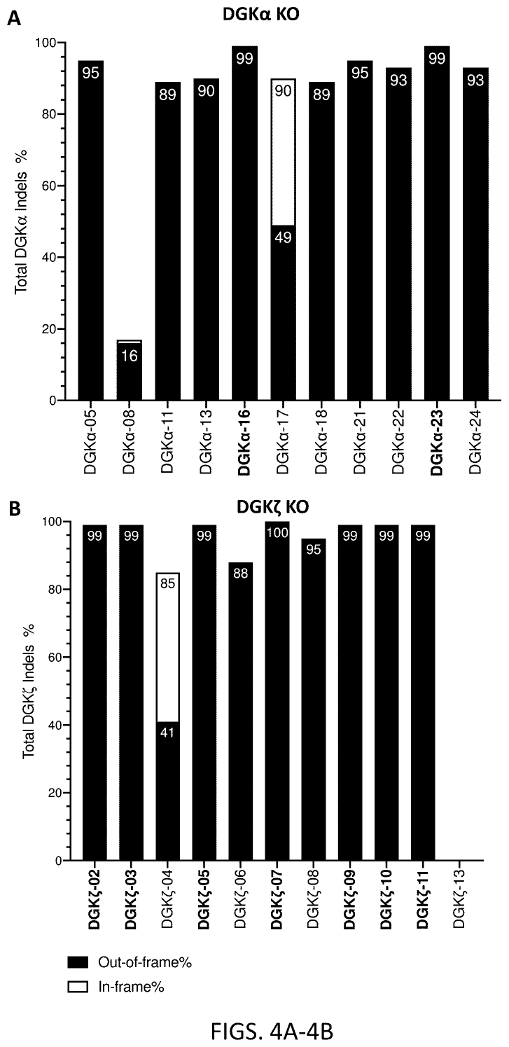

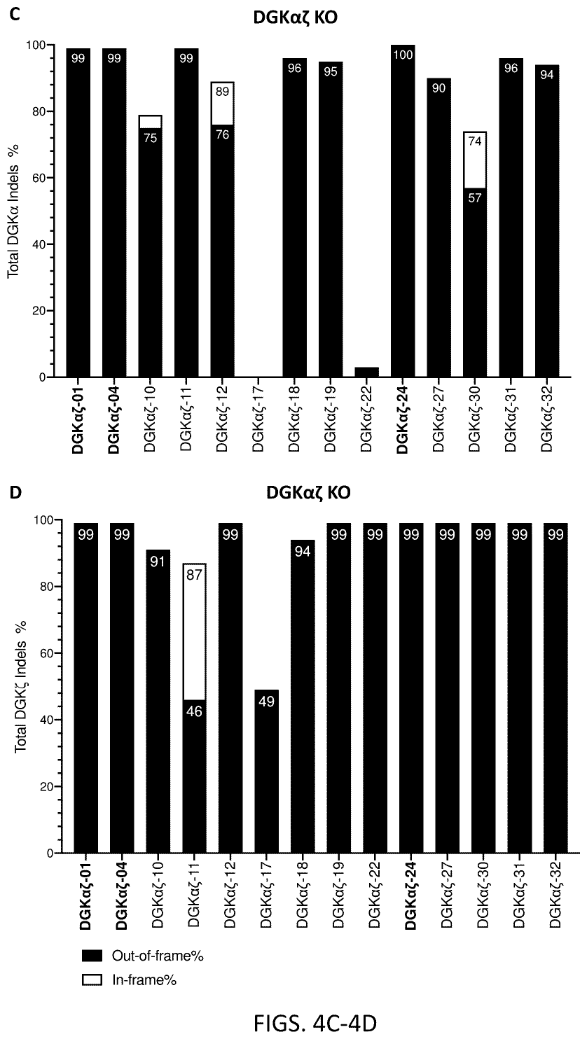

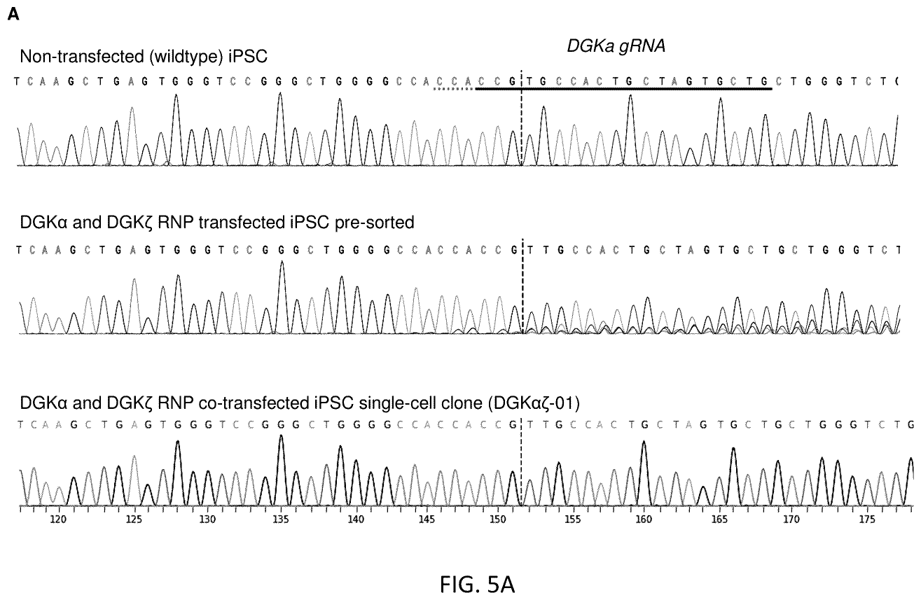

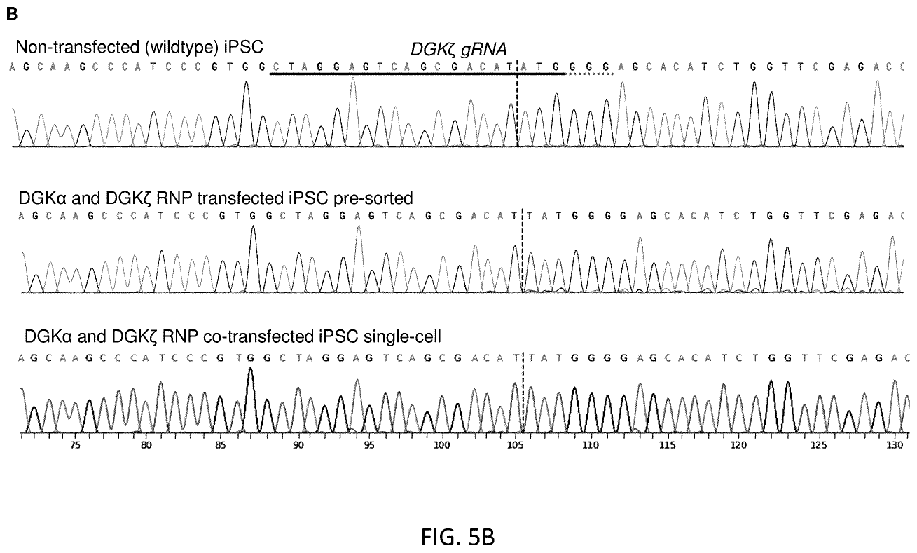

For single-cell cloning, firstly a 96-well plate was coated with CellAdhere™ Laminin-521 in PBS for 2 hrs at 37° C. The DGKα (SEQ ID NO: 3) and/or DGKζ (SEQ ID NO: 11) RNP transfected iPSCs were sorted and seeded into the 96-well plates at the density of 1 cell/well and cultured until colonies were formed (e.g., 5 to 9 days). Transfected iPSCs were lifted as single cells using Accutase® and subsequently counted, washed and resuspended for staining with relevant antibody cocktails, if required. Cells were stained at 4° C. for 15 minutes prior to sorting with the BD FACSAria™ Fusion. PI staining was used for dead cell exclusion. Cells were gated to remove debris, doublets and dead cells, and were subsequently sorted into a 96-well plate. The plate was placed immediately into a 37° C. cell incubator for 48 hrs and daily media changes were performed until the cells were suitable for passaging. After single-cell cloning, the genotypes of single-cell clones were analysed using Sanger sequencing and ICE assay. The single-cell clones with out-of-frame frequency between 99% to 100% were selected as KO iPSC single-cell clones (FIGS. 4A-4D). The genotypes of these selected clones were further compared with the wildtype Sanger sequencing traces. A representative genotyping result of the DGK double gene KO clone (DGKαζ KO iPSC single-cell clone 01) from DGKα and DGKζ RNP co-transfected iPSCs is shown. The pre-sorted DGKα and DGKζ RNP co-transfected iPSCs were purified after single cell cloning, evidenced by removing of the heterogeneous mix of bases downstream of the cut site, and identification of thymine (T) insertion (+1) in the single clone as compared to the non-transfected (wildtype) cells (FIGS. 5A-5B). In summary, these results demonstrated that the DGKα gRNA and DGKζ gRNA are capable of introducing indels into DGK genes and results in frame-shift mutation iPSCs efficiently, to generate DGK KO iPSCs.

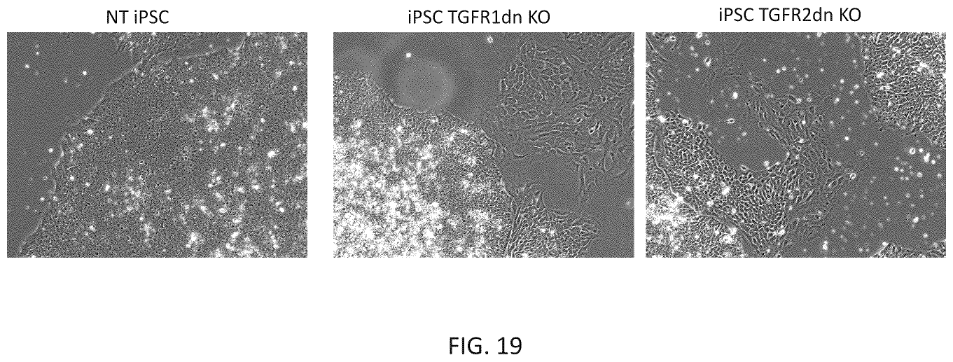

TGFβ exerts systemic immune suppression and inhibits host immunosurveillance, and is considered to be one of the major factors of the immunosuppressive microenvironment in tumor. Knocking-out of TGFβ receptors and direct inhibition of TGFβ signaling in embryonic stem cells results in loss of pluripotency (Watabe and Miyazono, Roles of TGF-beta family signaling in stem cell renewal and differentiation, Cell Research, January 2009, Vol. 19(1), pp. 103-15). We generated a dominant negative TGFβ1 and TGFβR2 iPSCs using CRISPR/CAS9 technology (as described in patent PCT/AU2020/051243 incorporated herein by reference). However, TGFβR1 and TGFβR2 dominant negative mutation in iPSCs lost their pluripotency and started spontaneously differentiating after gene editing (FIG. 19). This result indicates gene knockout or direct inhibition of TGFβ receptors in iPSCs cannot generate a source cell for immune cell therapy.

### Example 3—Generation of TAG-72 CAR Clones/DGK KO Pre-Sorted iPSCs and TAG-72 CAR/DGK KO iPSC Single-Cell Clones

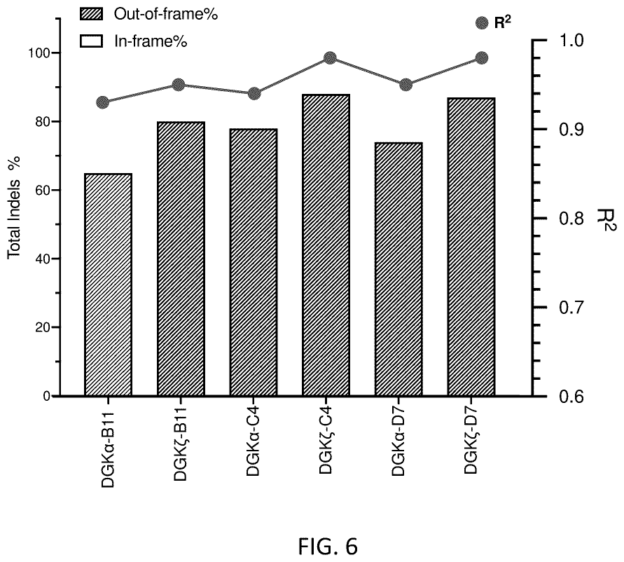

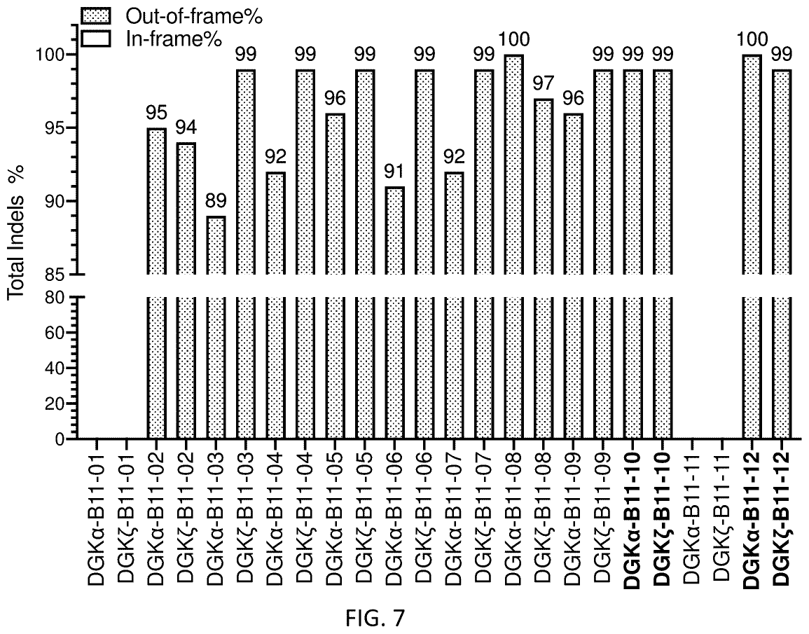

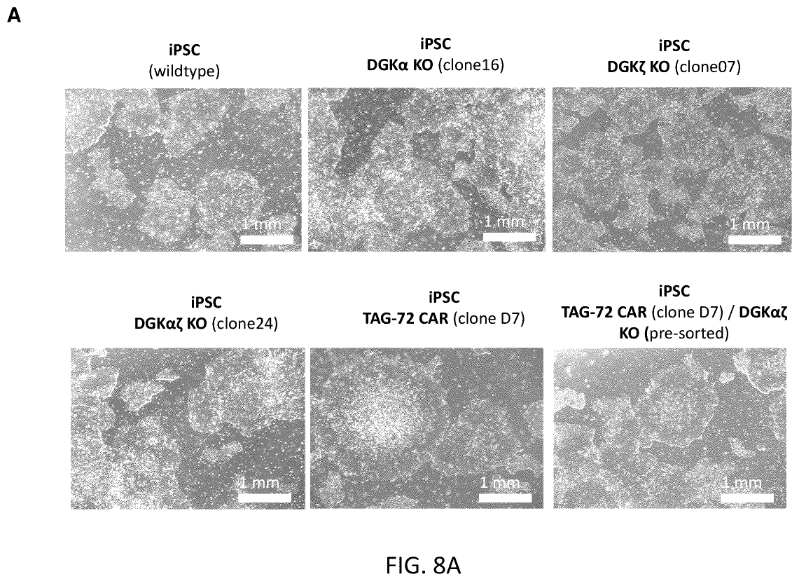

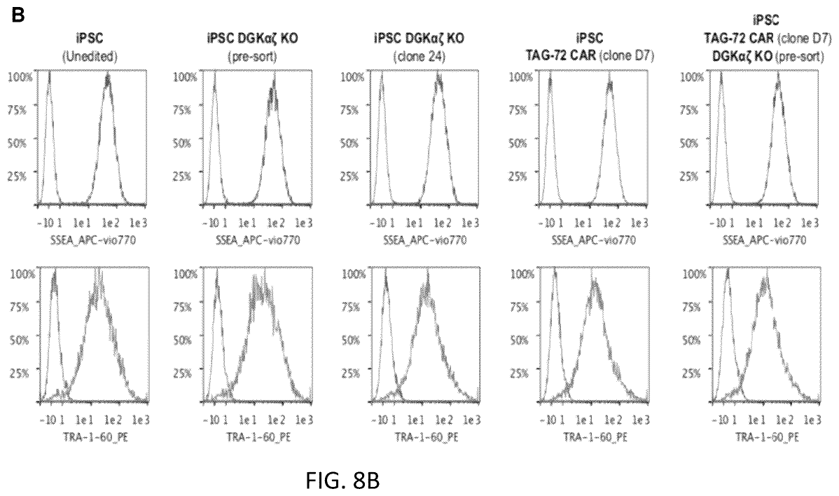

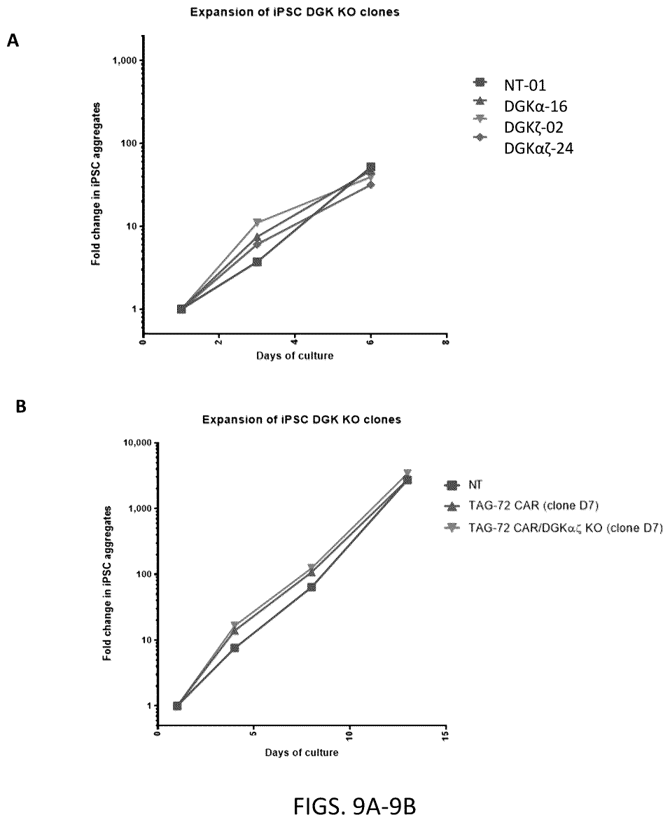

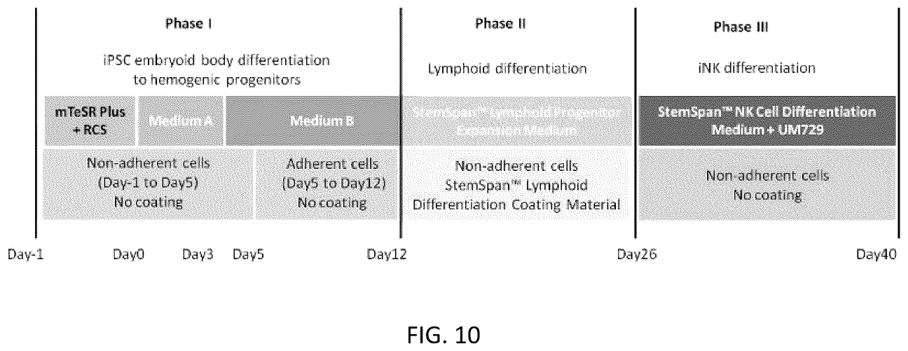

To generate TAG-72 CAR clones/DGK KO iPSCs pre-sorted, TAG-72 CAR iPSC single-cell clones were first created as described in Example 1. RNP complexes formed by representative gRNAs were then transfected into TAG-72 CAR iPSCs using the Lonza 4D-Nucleofector™ system as described in Example 2. ICE analysis results show that DGKα gRNA and DGKζ gRNA are capable of introducing indels into DGK genes and result in frame-shift mutations in TAG-72 CAR iPSCs at a high frequency (FIG. 6). Moreover, TAG-72 CAR/DGK KO iPSC single-cell clones can also be derived using the method mentioned in Example 2 (FIG. 7).

### Example 4—Differentiation of DGK KO iPSC Single-Cell Clones and TAG-72 CAR Clones/DGK KO Pre-Sorted iPSCs into CD34+ Cells

Stem cells, such as induced pluripotent stem cells (iPSCs), can self-renew indefinitely and differentiate into various cell types including hematopoietic stem cells (HSCs) and immune cells.

CD34+ cells can be made using a range of published methods, such as those described in U.S. Pat. No. 9,260,696 B2 (Kaufman, Knorr), by Li et al. (Stem Cell, 23 (2018) 181-197), or using commercially available culture systems such as STEMdiff™ Hematopoietic Kit (STEMCELL Technologies). In one variation of these methods, directing differentiation of iPSCs towards CD34+ hemogenic progenitor cells is facilitated through Embryoid Body (EB) formation using an AggreWell™400 6-well plate (STEMCELL Technologies). The AggreWell™ plate can be pre-treated with Anti-adherence Rinsing Solution (STEMCELL Technologies) and washed once with warm mTeSR Plus™ before use. mTeSR Plus™ with RevitaCell™ Supplement are then added at 2.5 mL per well. iPSC cultures are propagated until 70% confluency is reached, at which time iPSCs are dissociated into single cells using Accutase® and reseeded into an AggreWell™ plate and medium topped up with mTeSR Plus™ with RevitaCell™ Supplement to a total of 5 mL per well. The AggreWell™ plate is then centrifuged at 100×g for 3 minutes to draw cells into each microwell, and incubated at 37° C., 5% CO₂ for 24 hrs. After the incubation period, the medium is removed with a serological pipette and 5 mL of STEMdiff™ Hematopoietic Medium A (STEMCELL Technologies) is added per well. This marks Day 0 of hemogenic specification. On Day 2, a half medium change is performed, in which 2.5 mL of Medium A is removed and replaced with 2.5 mL of fresh Medium A. On Day 3 a complete medium change is performed, in which Medium A is removed and replaced with 5 mL of STEMdiff™ Hematopoietic Medium B (STEMCELL Technologies). On Day 5, EBs are collected by firmly pipetting culture medium against the surface of each well to dislodge EBs. The suspension is passed through a 40 m filter. The filter is then inverted and the EBs collected on the surface of the filter washed with 2.5 mL Medium B into a fresh 50 mL plastic tube. EBs are then seeded into wells of a non-tissue culture treated 6-well plate in 2.5 mL of Medium B. On Day 7, 2.5 mL of Medium B is added per well. On Days 9 and 11, a half medium change is performed with Medium B, as outlined above. On Day 12, both adherent and non-adherent cell fractions are collected and CD34+ cells sorted using the CD34 MicroBead Kit (Miltenyi Biotec) according to manufacturer's instructions.

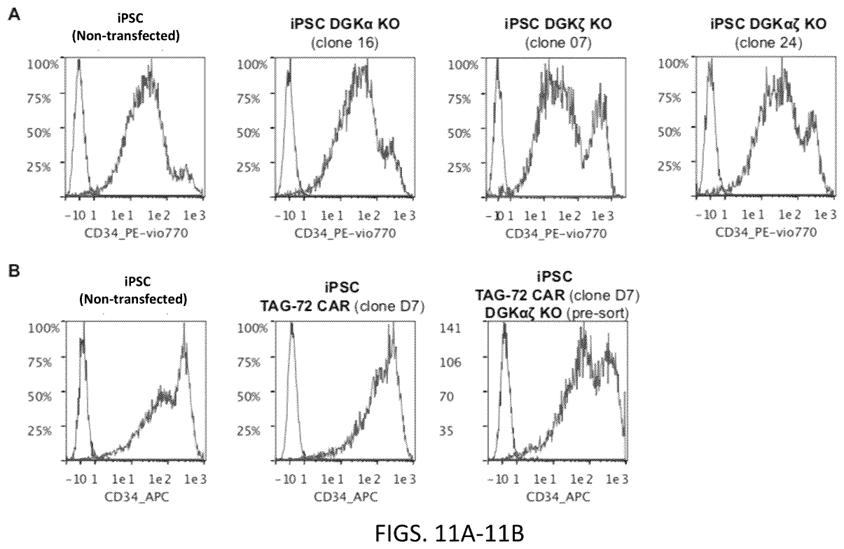

TAG-72 KI, DGK KO and cloning was performed as described in the above examples. DGK KO in iPSC single-cell clones and in TAG-72 CAR clones/DGK KO pre-sorted iPSCs were then differentiated into CD34+ cells. Sort purity and hemogenic progenitor profiles of the created CD34+ cells were determined through flow cytometry for CD34 expression. Flow cytometric analysis of hemogenic progenitors specified from DGK KO iPSCs or TAG-72 CAR/DGK KO iPSCs shows comparable expression of CD34 to non-transfected controls, indicating that either DGK KO alone or the combination of TAG-72 CAR KI and DGK KO does not impact differentiation of iPSCs to CD34+ hemogenic progenitors (FIGS. 11A-11C).

### Example 5—Differentiation of DGK KO CD34+ (with or without TAG-72 CARs) into iNK Cells

iNK cells can be generated using a range of published methods, such as those described in U.S. Pat. No. 9,260,696 B2 (Kaufman, Knorr), by Li et al. (Stem Cell, 23 (2018) 181-197), or using the commercially available culture system StemSpan™ NK Cell Generation Kit (STEMCELL Technologies). In one variation of these methods, CD34+ hemogenic progenitors sorted (as for example described in Example 4), derived from either DGK KO iPSC single-cell clones or TAG-72 CAR clones/DGK KO iPSCs pre-sorted are reseeded onto plates coated with StemSpan™ Lymphoid Differentiation Coating Material (STEMCELL Technologies) in StemSpan™ Lymphoid Progenitor Expansion Medium (STEMCELL Technologies). This marks Day 0 of NK Cell specification. On Day 3, one volume of Expansion Medium is added to the culture vessel. Half medium changes are performed on Days 7 and 10 with fresh Expansion Medium. On Day 14, non-adherent lymphoid progenitor cells are collected and centrifuged at 300×g for 5 minutes. Cells are then be resuspended in StemSpan™ NK Cell Differentiation Medium supplemented with 1 μM UM729. On Day 17, one volume of NK Differentiation Medium is added to the culture vessel. Half medium changes are performed on Days 21 and 24 with fresh NK Differentiation Medium. Differentiation to NK cells is complete by Day 28. The efficiency of NK cell differentiation can be enhanced by supplementing media with combinations of cytokines, including IL-15, FLT3 and IL-7.

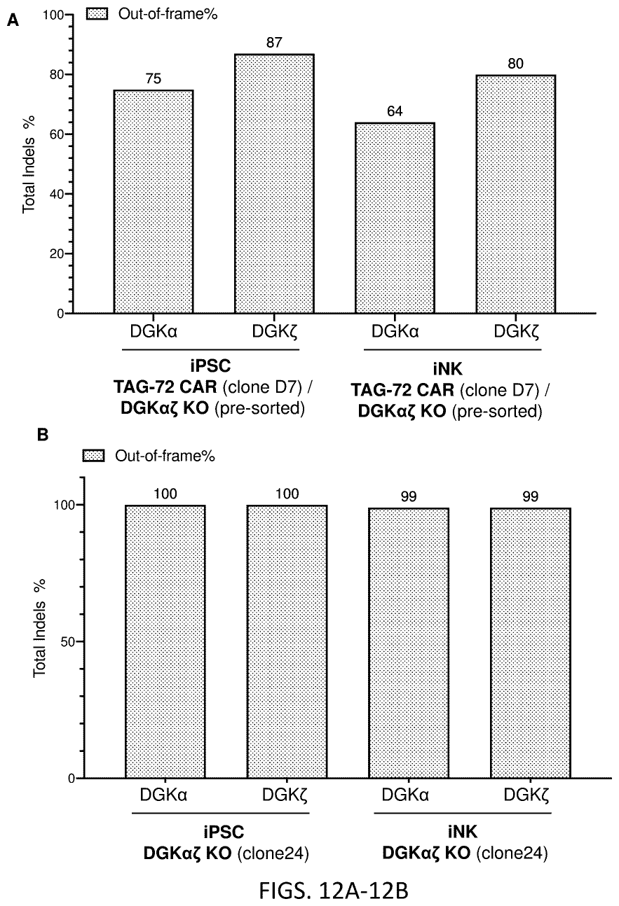

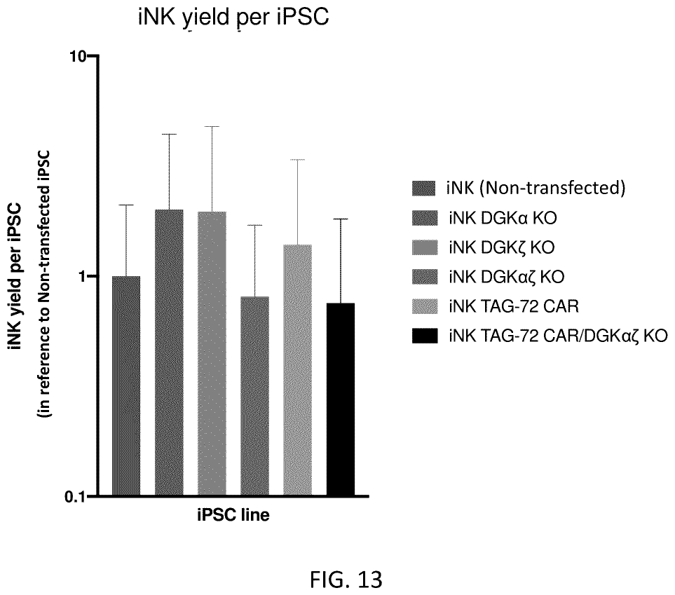

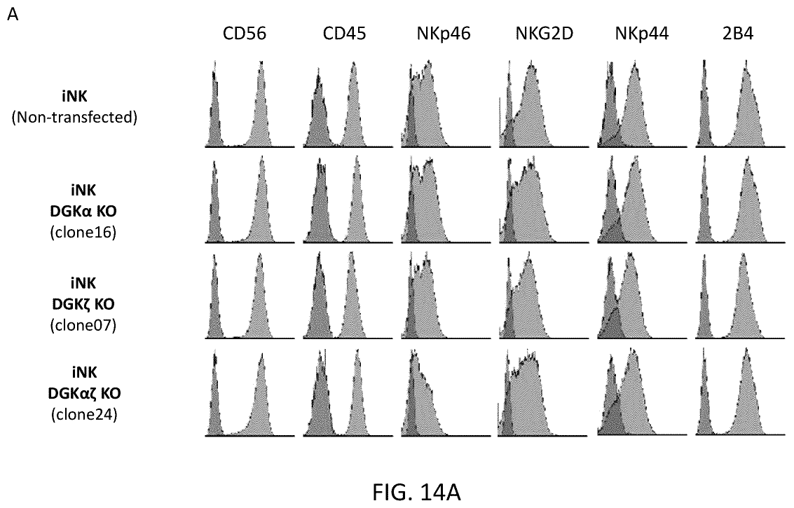

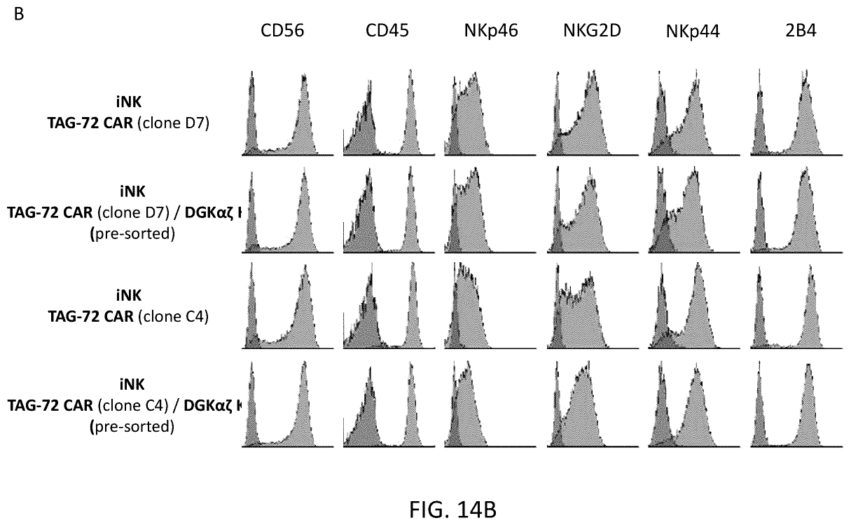

DGK KO CD34+(with or without TAG-72 CARs) were differentiated into iNK cells and analysed for DGK KO, NK cell phenotype, and NK cell function. ICE analysis confirmed that the iNKs±TAG-72 CAR possessed indels at both the a and (loci at percentages comparable to their parental DGK KO iPSCs±TAG-72 CAR (FIGS. 12A-12B). Furthermore, it was shown that single or double gene DGK KO in iPSCs with or without TAG-72 CAR KI, does not impact the yield of NK cells obtained from the differentiation process (FIG. 13). Flow cytometric analysis for the NK cell markers CD56, CD45, NKp46, NKG2D, NKp44, and 2B4 showed comparable expression levels between non-transfected and transgenic iNKs, indicating that single or double DGK KO, in the presence or absence of TAG-72 CAR KI, does not have an effect on the phenotype of iNK cells (FIGS. 14A-14B).

### Example 6—In Vitro Function of TAG-72 CAR KO iNK Cells and KO iNK Cells

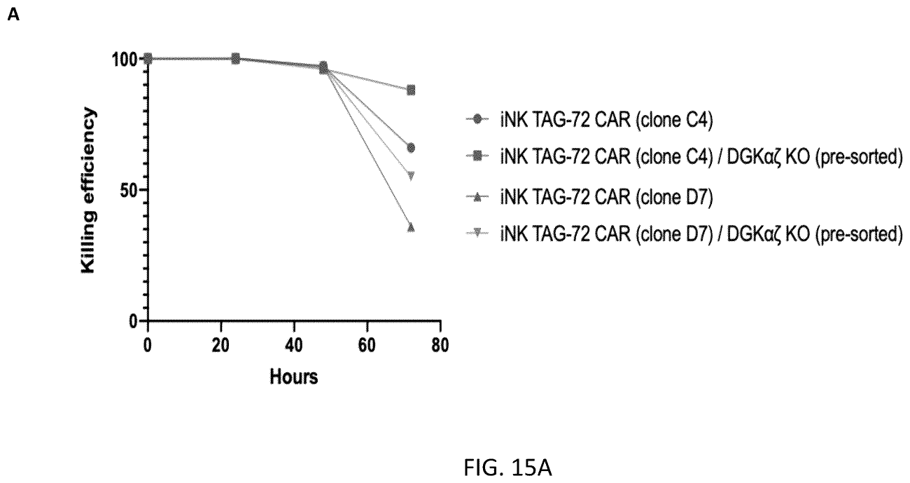

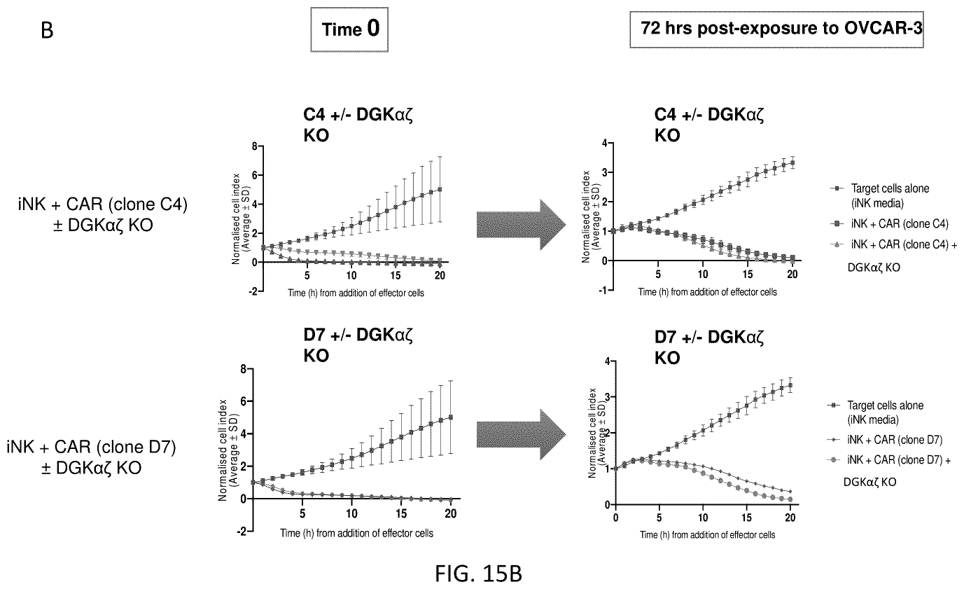

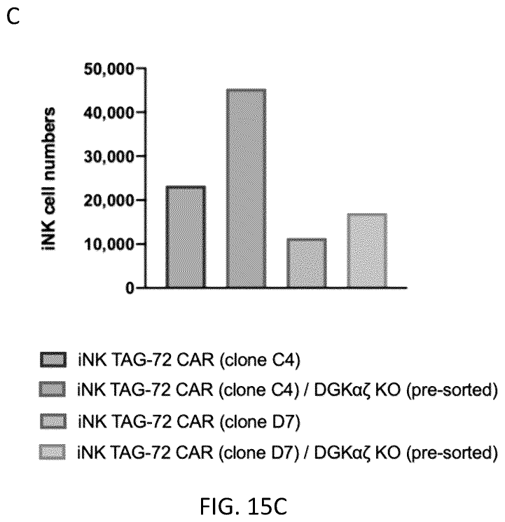

The real-time cell monitoring system (xCELLigence®) was employed to determine the killing efficiency of iNK cells in vitro. Target cells at 10,000/100 μL (for example the ovarian cancer cell line OVCAR-3) were resuspended in culture media (for example, RPMI-1640 basal media) supplemented with 20% fetal calf serum and bovine insulin and deposited into a Real Time Cell Analysis microtitre ePlate compatible with the xCELLigence® system. Target cells were maintained at 37° C., 5% CO₂ for 3-20 h to allow for cellular attachment. Following attachment of target cells, iNK effector cells with or without TAG-72 CAR or DGKαζ KO were added at an effector to target (E:T) ratio of 1:1. All co-cultures were maintained in optimal growth conditions for at least 20 hrs. Throughout, cellular impedance was monitored; a decrease in impedance is indicative of target cell detachment and ultimately cell death. DGKαζ KO was performed on iPSC clones either with or without TAG-72 CAR insertion as described in Examples 1, 2 and 3. These cells were then sequenced and differentiated into iNK cells. iNK cells with and without TAG-72 CAR and DGKαζ KO were placed into a xCELLigence® assay (as described above) targeting OVCAR-3 cells at an effector to target ratio of 1:1, to assess baseline iNK function prior to the prolonged exposure to OVCAR-3 cells. For the antigen exposure assay, OVCAR-3 cells (routinely cultured following ATCC recommendations) were seeded at 8×10⁴ cells per well in 12 well plates and left for 6 hrs to attach to the plate. 8×10⁵ iNK cells were then placed into OVCAR-3 containing wells and left to incubate for a total of 72 hrs. Every 24 hrs (i.e. 24 hrs, 48 hrs and 72 hrs of co-culture), the non-adherent fraction of the culture containing iNK cells and any dead OVCAR-3 cells were collected, centrifuged at 300×g for 10 minutes, resuspended in fresh iNK culture medium, and placed into a well containing untouched OVCAR-3 cells. The remaining OVCAR-3 cells which were not killed by the iNKs (i.e. the remaining adherent fraction) was enzymatically detached using Trypsin-EDTA solution and counted using MUSE® Cell Counter. These cell counts were utilised to calculate the iNK killing efficiency as shown in FIG. 15A. iNK cells at the end of the antigen exposure assay were also assessed for cytotoxic function using xCELLigence® as previously described.

iNK cells with CAR KI and DGKαζ KO showed enhanced long-term cytotoxic function against OVCAR-3 tumor cells in vitro (FIG. 15A). The effect of including the DGKαζ KO with the CAR was revealed after 72 hrs of repeat exposure to cancer cell lines. These data demonstrated the novel function of including DGKαζ KO in iPSCs providing functional benefit in iNK cells (FIG. 15A). After 72 hrs of repeat exposure to OVCAR-3, the time taken to eliminate target cells was increased (15-20 hrs) compared to baseline (approximately 5 hrs) (FIG. 15B). However, when comparing the function of iNK cells which were exposed to OVCAR-3 cells for 72 hrs before killing capacity was assessed, TAG-72 CAR/DGKαζ KO iNK demonstrated an improvement in function (FIG. 15B). Additionally, the total number of iNK cells that survived the prolonged antigen exposure assay was improved in TAG-72 CAR/DGKαζ KO iNK clones compared to their respective non-transfected iNK controls (FIG. 15C). Taken together, this indicates that DGKαζ KO is important for iNK survival and enhancement cytotoxic function against tumor cells.

### Example 7—In Vitro Function of DGKαζ KO iNK Cells

As previously described in Example 6 above, the real-time cell monitoring system (xCELLigence®) was employed to determine the killing efficiency of iNK cells in vitro. For the prolonged antigen exposure assay, OVCAR-3 cells (routinely cultured following ATCC recommendations) were seeded at 2.5×10⁴ cells per well in 48-well plates and left for 4-5 hrs to attach to the plate. 5×10⁵ iNK cells (E:T 20:1) were then placed onto OVCAR-3 wells and left to incubate for 24 hrs. After 24 hrs, the non-adherent fraction of the culture containing iNK cells, as the only viable cells, was collected, centrifuged at 300 g for 10 minutes, resuspended in fresh iNK culture medium, and counted using the Guava® MUSE® Cell Analyser. These remaining iNKs were then placed into a new well containing OVCAR-3 cells, which were freshly seeded 4 hrs prior. The remaining OVCAR-3 cells which were not killed by the iNKs were lifted via Trypsin-EDTA and counted using the Guava® MUSE® Cell Analyser. This process was repeated at each 24 hr time interval for 5 days total. iNK cells at the end of the antigen exposure assay were also placed into an xCELLigence® assay to demonstrate the effect of iNK cytotoxic function after 120 hrs of exposure to OVCAR-3 cells. At the experiment end-point, a sample of iNK cells was also subject to flow cytometry analysis to assess expression of the proliferative nuclear marker, Ki67.

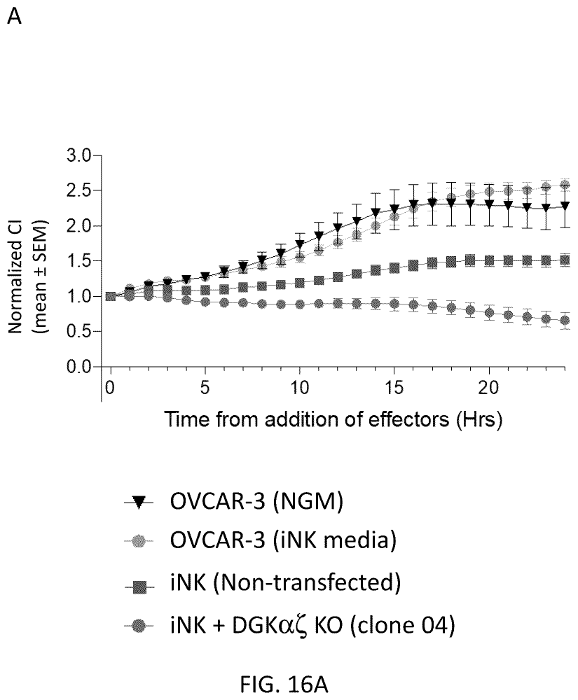

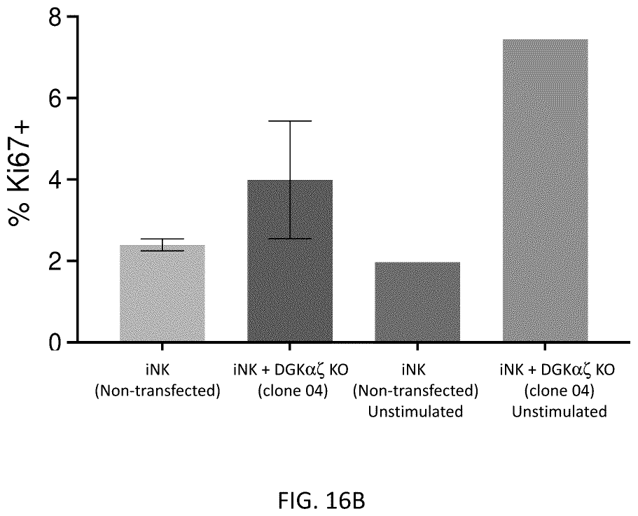

iNK cells with DGKαζ KO showed enhanced long-term cytotoxic function against OVCAR-3 tumour cells in vitro (FIG. 16A). After 120 hrs of repeated antigen exposure, the DGKαζ KO iNK cells demonstrated enhanced killing capacity compared to non-transfected iNK cells, as measured by the xCELLigence® assay (FIG. 16A). These data demonstrate the novel function of including DGKαζ KO in iPSCs providing functional benefit in iNK cells (FIG. 16A). Furthermore, flow cytometry analysis after repeated antigen exposure suggested an increased proportion of cells expressing the proliferative marker Ki67 in the DGKαζ KO iNKs compared to non-transfected iNKs (FIG. 16B). This trend toward increased Ki67 expression hints towards an improved proliferative capacity in DGKαζ KO iNKs.

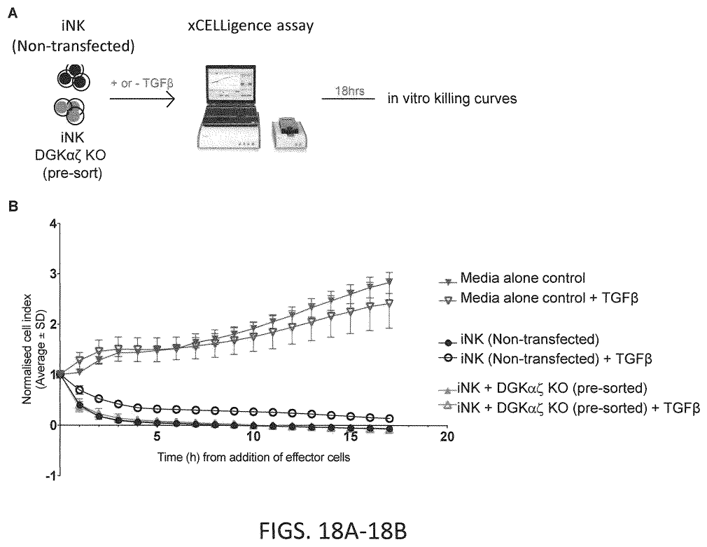

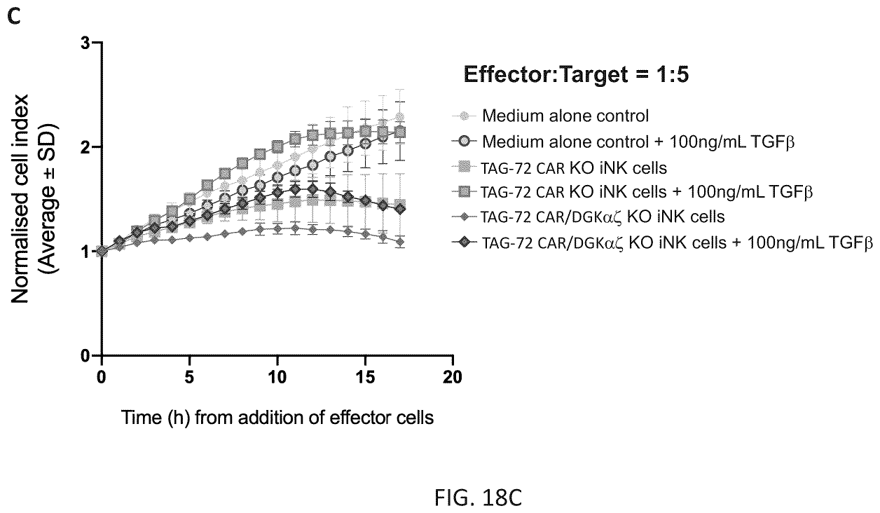

The functional advantage of DGK KO in iNK cells at the tumour micro-environment was modelled via in vitro cytotoxic functional assays described in Example 6, in the presence of TGFβ. As a common phenomenon, solid tumour microenvironments are intensively infiltrated with a range of immuno-checkpoint inhibitors and soluble factors, such as TGFß, which causes tumour escape and resistance to immune cells (Alsina-Sanchis, Elisenda et al., TGFβ Controls Ovarian Cancer Cell Proliferation, International journal of molecular sciences, 2017-07-30, Vol. 18 (8), p. 1658). For example, previous studies have shown TGFß can suppress nature killer cell functions via multiple signalling pathways and result in the down-regulation of NK activating receptors, such as natural killer group 2 member D (NKG2D) (reviewed by Yang, Li et al., TGF-β and immune cells: an important regulatory axis in the tumor microenvironment and progression, 2010, Vol. 31 (6), p. 220-227) and herein compromised killing capacities. The response of non-transfected iNK cells and DGKαζ KO iNK cells targeted against OVCAR-3 at 2:1 effector to target ratio, in either standard NK culture media or supplementation of TGFβ at 10 ng/mL was investigated in FIG. 18A-18B. OVCAR-3 cell growth was monitored in culture media alone with or without TGFβ (without iNK cells) as internal controls within the assay. In this study, the inclusion of DGKαζ KO in iNK cells was able to resist or avoid suppressive effects that TGFβ had on cytotoxic function against the OVCAR-3 cells, compared to non-transfected iNK cells which had a reduced killing response within TGFβ supplemented media. As direct TGFβ KO cannot be performed on iPSC without causing spontaneous differentiation (shown in FIG. 19), the ability for DGKαζ KO to enable downstream advantages of iNK cells through minimising suppressive effects within TGFβ associated pathways demonstrates a novel advantage of DGKαζ KO in iPSC and iNK cells.

The functional advantage of DGKαζ KO was further assessed in vitro in combination with the TAG-72 CAR. In this assay, the TAG-72 CAR (clone D7) iNK cells with or without DGKαζ KO, were used as effector cells in an xCELLigence® assay against OVCAR-3 cells under more challenging conditions, where the effector to target ratio was reduced to 1:5 and the supplementation of TGFβ in NK media was increased to 100 ng/mL. In FIG. 18C, the immune suppressive impact of TGFβ on effector cells was greater in TAG-72 CAR iNK cells than TAG-72 CAR/DGKαζ KO iNK cells. The addition of TGFβ at 100 ng/mL abrogated the cytotoxic function of TAG-72 CAR iNK cells against OVCAR-3 cells. Under the same conditions, TAG-72 CAR/DGKαζ KO iNK cytotoxicity was only reduced slightly with the addition of TGFβ. These data support that TAG-72 CAR iNK cells are sensitive to immunosuppressive factors, including TGFβ, that mimic aspects of the endogenous tumour microenvironment. The inclusion of DGKαζ KO into iPSC cells and downstream retention through to iNK cells following differentiation reduces the impact of TGFβ on iNK cytotoxic function against the OVCAR-3 cells.

### Example 8—In Vivo Function of DGKαζ KO iNK Cells

For this model, human tumor cell lines were grown on the flank of NSG mice by subcutaneously injecting approximately 1×10⁷ human-derived TAG-72 positive OVCAR-3 cancer cells into the flanks of 6 to 10-week-old mice. Within 6-7 weeks, fully formed ˜100 mm³ tumors developed at the injection site. Once tumors reached this volume, the groups were randomized for treatment. iNK cells derived from either unedited (i.e. non-transfected) iPSC sources or DGKαζ KO iPSC cells, were administered to the mice intravenously at 1×10⁶ NK cells per injection. Prior to injection, iNK cells were cultured in vitro in normal NK expansion conditions for 7 days. No exogenous cytokines were co-administered with the iNK cells to the mice. The tumor volume, body weight and clinical score were monitored after iNK cell infusion. Mice with tumor size from 800 mm³ to >1000 mm³, significant weight loss or poor clinical score were culled, according to animal ethics approvals.

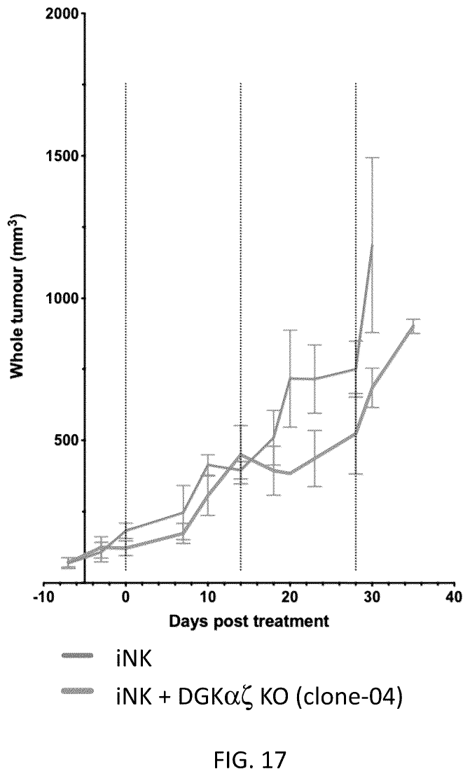

Administration of human cytokines has been demonstrated to support human NK cell survival and function in NSG mice. However, in this study no human cytokines were co-administered with the iNK cells and the iNK cells still demonstrated ant-tumor activity. Mice treated with iNK cells with DGKαζ KO demonstrated reduced tumour size during multiple points through the assay and showed an improvement in survival (FIG. 17).

### Example 9—In Vitro and In Vivo Characterization of TAG-72 CAR and TAG-72 CAR/DGKαζ KO iNK Cells

Prior to in vivo administration, effector cell preparations were pre-evaluated by a set of optimised in vitro assays. This included flow cytometric analysis of NK cell purity, phenotypic characterisation of relevant NK stimulatory receptors, TAG-72 CAR expression, DGKαζ KO status, as well as in vitro OVCAR-3 killing activity.

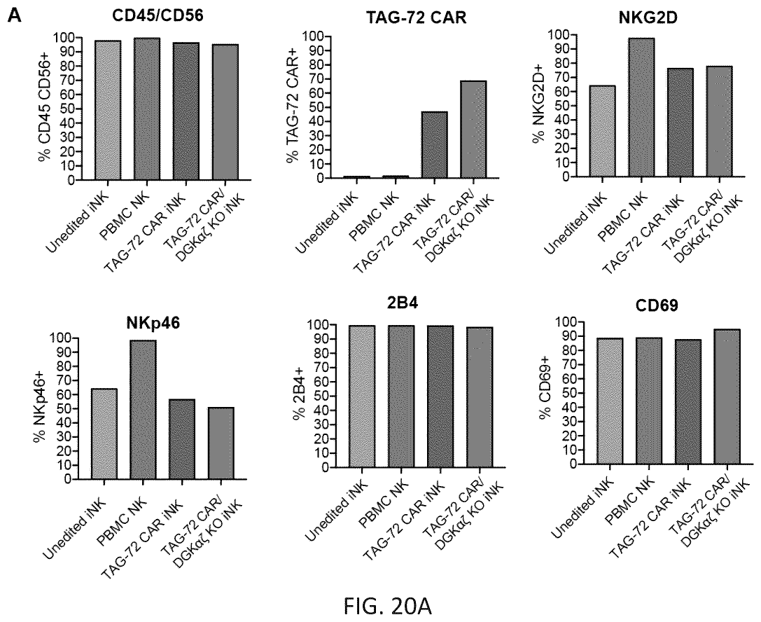

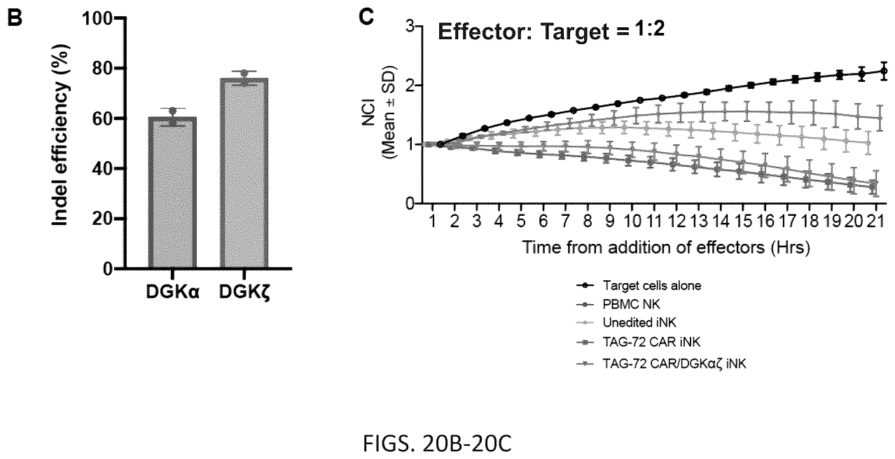

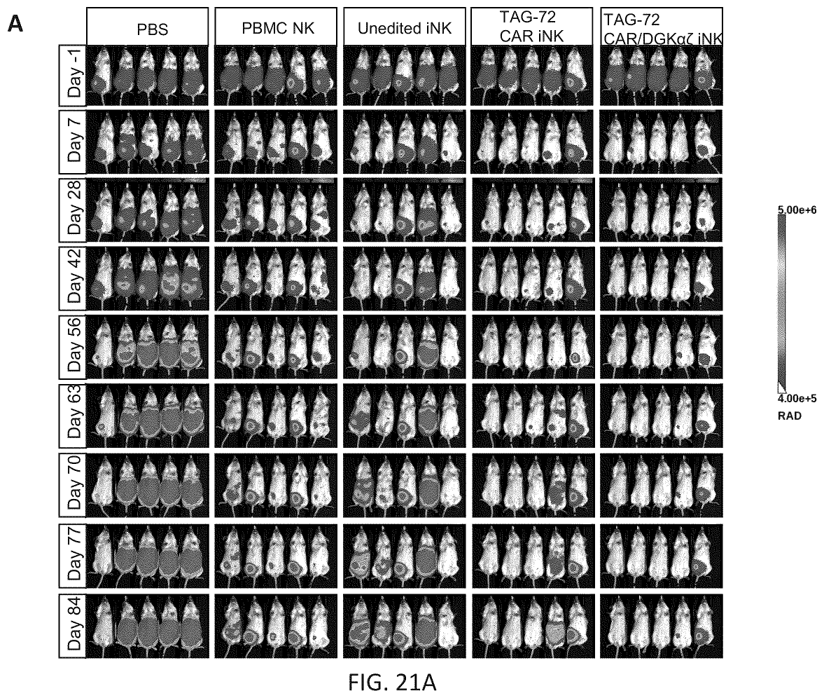

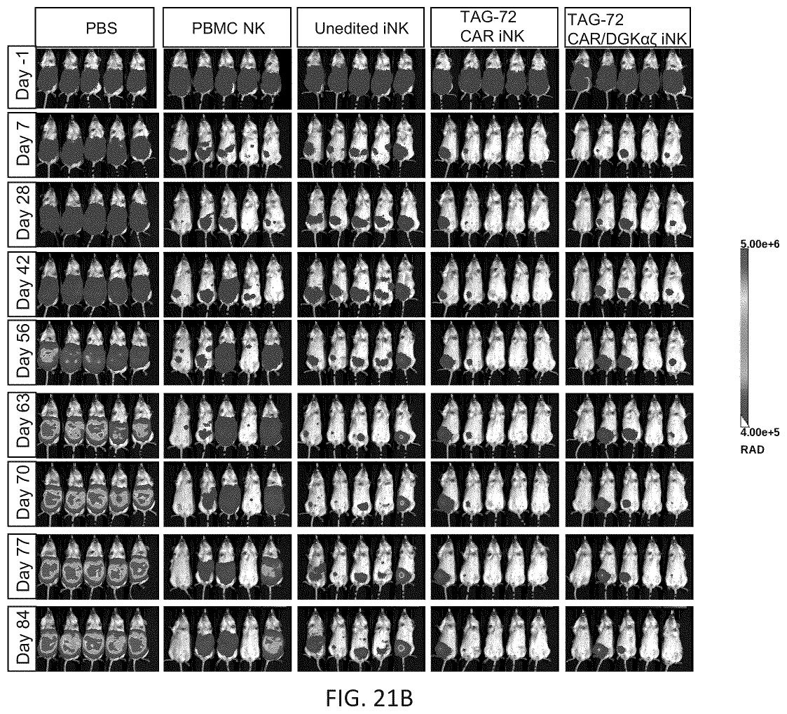

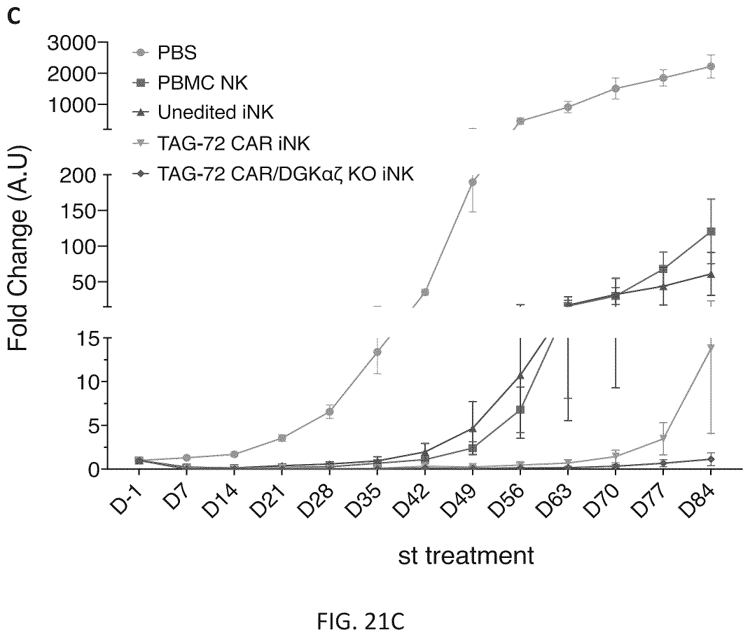

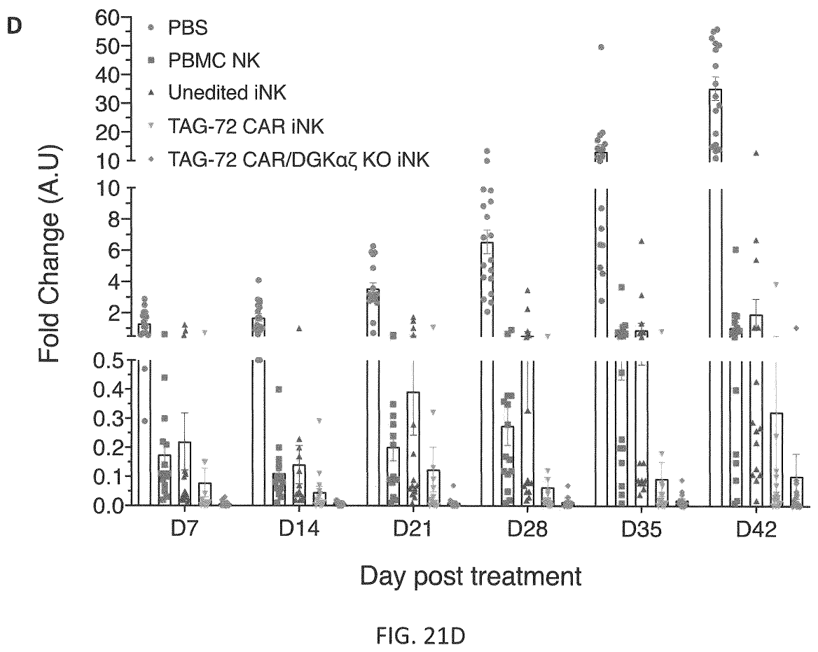

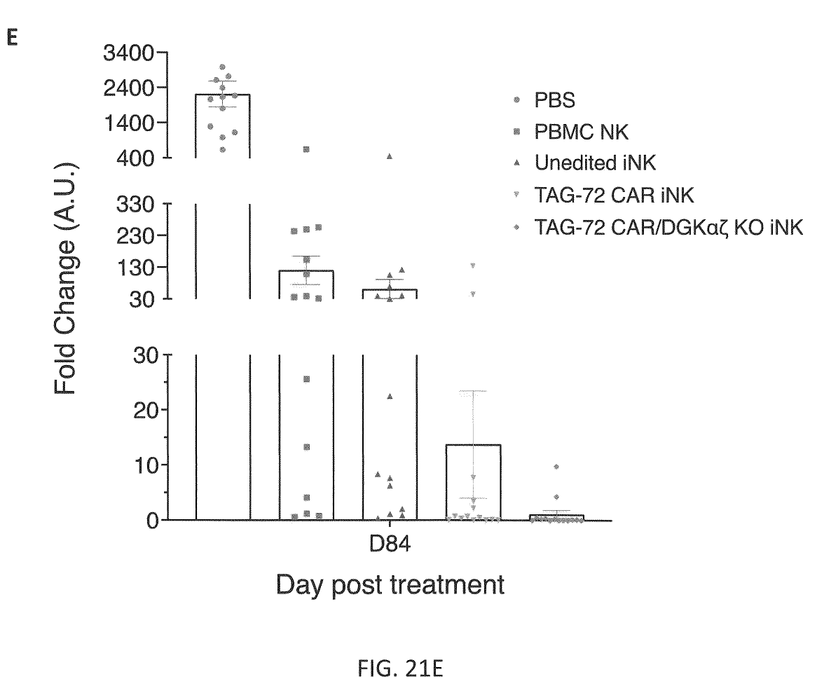

Flow cytometry analysis of TAG-72 CAR/DGKαζ KO iNK cells demonstrated a high proportion of double-positive CD45+ CD56+ cells, suggesting a relatively pure population of NK cells (FIG. 20A). Of these double-positive TAG-72 CAR/DGKαζ KO cells, more than 60% expressed the TAG-72 CAR, suggesting that CAR retention was not impacted by the manufacturing and editing process (within an acceptable range). The NK stimulatory receptors NKG2D, NKp46, and 2B4, as well as the early activation marker CD69, were also expressed at significant levels on TAG-72 CAR/DGKαζ KO cells, and expression was comparable to that observed in TAG-72 CAR iNKs (FIG. 20A). Functional assessment using xCELLigence® impedance-based technology demonstrated that TAG-72 CAR/DGKαζ KO iNK cells enhance OVCAR-3 cell killing compared to unedited iNKs and NK cells isolated from healthy adult donor peripheral blood mononuclear cell (PBMC), at both Effector: Target ratios of 1:2 and 1:1 (FIG. 20C shows the 1:2 ratio, data for the 1:1 ratio is not presented). Furthermore, TAG-72 CAR/DGKαζ KO iNKs demonstrated similar killing capacity relative to TAG-72 CAR alone iNKs. The ICE tool, developed by Synthego, was used to analyse DGKα and DGKζ indel efficiency (FIG. 20B). This demonstrated an indel efficiency of approximately 60% and 75% for the DGKα and DGKζ loci, respectively, suggesting that the cytotoxic effects observed both in vitro (FIG. 20A-20C) and in vivo (FIG. 21A-21E) were largely mediated by cells containing the DGKαζ gene KO.

A luciferase-based bioluminescent imaging (BLI) xenograft model was used to determine the in vivo efficacy of TAG-72 CAR and TAG-72 CAR/DGKαζ KO iNK cells, which were generated as described in Examples 4 and 5 above. The OVCAR-3 human ovarian cancer cell line was selected as the xenograft based on its known expression of the target antigen TAG-72, the efficient engraftment of OVCAR-3 cells into NSG mice and the sensitivity of OVCAR-3 cells to TAG-72 CAR and/or TAG-72 CAR DGKαζ KO iNK cells in vitro (see Example 6). The experimental details are as follows: four days prior to effector cell administration (Day ˜4), 8-week-old female immunocompromised NSG mice were injected intraperitoneally (i.p) with 2×10⁵ luciferase-labelled OVCAR-3 cells. One day prior to NK cell administration (Day −1), mice were measured for baseline tumour burden using an AMI-HTX animal imager (Spectral Instruments Imaging) and placed into comparable luciferase-expressing groups. On Day 0, 1×10⁷ cryostored iNK cells were thawed and injected i.p. into mice. To support the survival and expansion of the human iNK effector cells, all groups received 1×10⁴ U of interleukin (IL)-2 administered every 2-3 days (for 21 days) and/or 10 ng of IL-15 administered daily (for 7 days) in the first 3 weeks of the experiment. Tumor burden was monitored and quantified weekly by BLI until the end of the study (84 days).

In this model, mice treated with TAG-72 CAR iNKs or TAG-72 CAR/DGKαζ KO iNK cells demonstrated measurably lower tumour burdens compared to PBS, PBMC-NK controls and unedited iNK-treated mice over the 84-day observation period (FIG. 21A-C). From day 70, a progressive increase of bioluminescence signal, reflecting a growth in tumour cells, was observed in mice treated with TAG-72 CAR iNKs but not in TAG-72 CAR/DGKαζ KO iNK-treated mice (FIG. 21C). Analysis of individual timepoints revealed a strong trend suggesting that the inclusion of the DGKαζ KO in iNKs resulted in superior long-term in vivo efficacy, as reflected by a consistently lower mean tumour burden compared to TAG-72 CAR alone iNKs (FIG. 21D, E).

### Example 10—Differentiation of DGK KO CD34+ (with or without TAG-72 CARs) into iT Cells

iT cells can be generated using a range of published methods, such as those described utilising genetically edited mouse stroma support cells (OP9) expressing delta like ligand 1 (DLL1) (Themeli et al., Generation of tumor-targeted human T lymphocytes from induced pluripotent stem cells for cancer therapy, Nat Biotechnol., October 2013, Vol 31(10), pp. 928-933) or delta like ligand 4 (DLL4) (Flippe et al., Rapid and Reproducible Differentiation of Hematopoietic and T Cell Progenitors From Pluripotent Stem Cells, Front. Cell Dev. Biol., 20 Oct. 2020, Vol 8, Article 577464), or via notch ligand signalling bound to a support structure in suspension (US20200399599A1, Method for generating cells of the T cell lineage), the presence of cytokines such as IL-7, FLT3, SCF, or using the commercially available culture system STEMdiff™ T Cell Kit (STEMCELL Technologies).

iPSC sources can be either non-edited or contain at least one of the following in any combination DGKα KO, DGKζ KO, DGKαζ KO or CAR knock-in, and can be either clonally derived, an enriched population, or derived from an impurified bulk gene-edited population. These iPSC sources are differentiated toward CD34+ cells. Pre-sorted CD34+ cells are reseeded onto plates coated with StemSpan™ Lymphoid Differentiation Coating Material (STEMCELL Technologies) in StemSpan™ Lymphoid Progenitor Expansion Medium (STEMCELL Technologies). This marks Day 0 of T cell differentiation. After 14 days, performing media changes twice per week, progenitor T cells are collected and placed into StemSpan™ T Cell Progenitor Maturation Medium (STEMCELL Technologies) for the recommended period of time. T cells are collected and then activated using either ImmunoCult™ Human CD3/CD28/CD2 T Cell Activator (STEMCELL Technologies) or Human T-Activator CD3/CD28 Dynabeads (Thermo Fisher) in CD8 SP T Cell Maturation Medium (STEMCELL Technologies) to create mature CD3+ T cells, with predominately CD8+ phenotype.

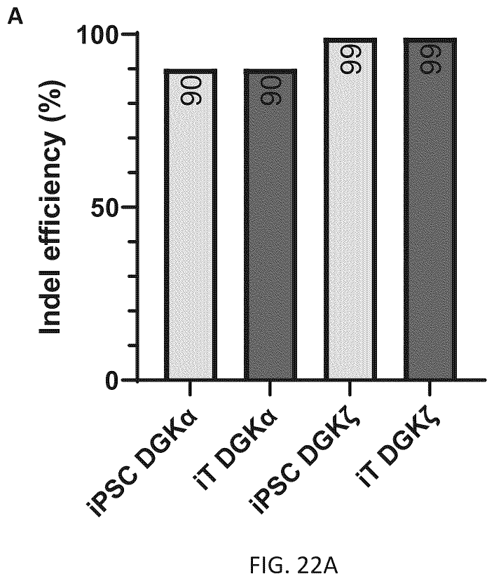

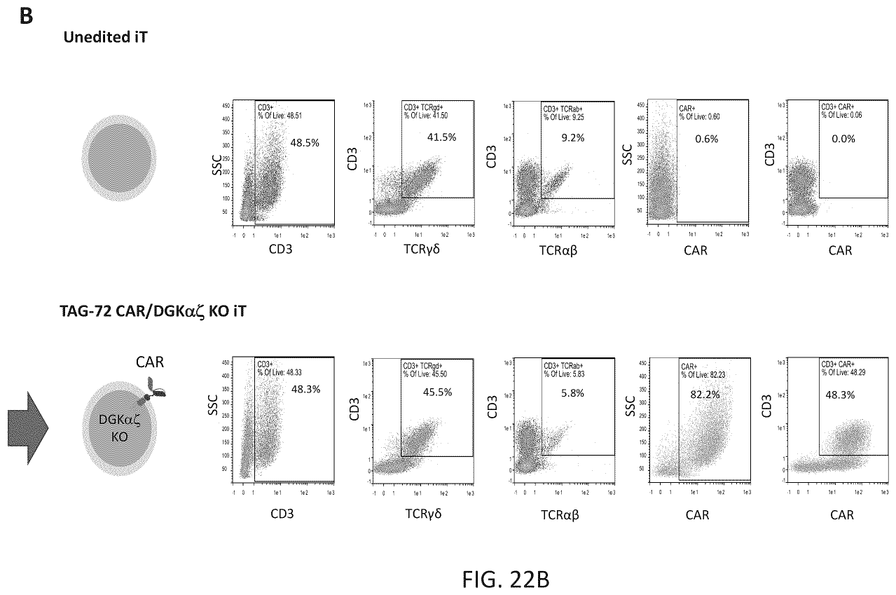

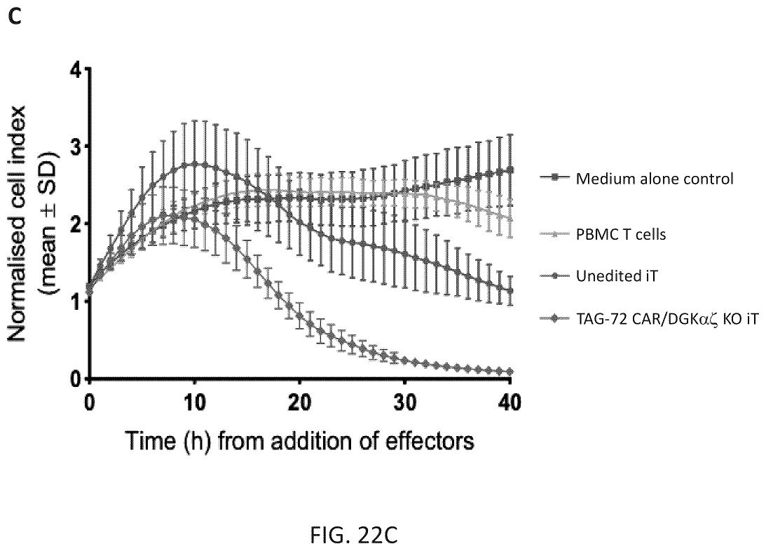

A CAR construct targeting TAG-72 was first knocked into a safe harbor locus in iPSCs using CRISPR/Cas9 gene editing, then both DGKα and DGKζ were knocked out using CRISPR/Cas9 (as described in Examples 1-2). Cells were cloned after gene editing, and DGKαζ KO and homozygote KI of the CAR were selected for differentiation (see Example 3). The indel efficiency in TAG-72 CAR iPSC enriched lines was 90% for DGKα and 99% for DGKζ (FIG. 22A). These iPSC lines were successfully differentiated to iT cells, where hallmark markers for bona fide T cells were characterised via flow cytometry (FIG. 22B). Importantly, populations of cells co-expressing CD3+ with TCRαρ and TCRγδ were obtained and at equivalent levels with or without CAR and DGKαζ KO. This supports the finding that the inclusion of the DGKαζ KO in iPSCs does not block development of iPSCs to iT cells. Co-expression of the CAR with CD3 further confirms that iT cells explicitly retain the CAR.

As shown in FIG. 22C, iPSC-derived TAG-72 CAR+DGKαζ KO iT cells show on-target CAR-mediated activity against the TAG-72 expressing ovarian cancer cell line OVCAR-3 as shown via xCELLigence® in vitro. Conversely, compared to OVCAR-3 controls, non-transfected iT cells and T-cells isolated from PBMC without the CAR construct do not show equivalent function. Of key importance, potent cytotoxic function is retained in iT cells which have been derived from iPSC clones with DGKαζ KO and CAR KI gene-editing, thus demonstrating functional CAR iT cells with DGKαζ KO.

### Example 11—In Vitro Function of TAG-72 CAR/DGK KO iT Cells in the Presence of TGFβ

iT cells can be generated as described in Example 10.

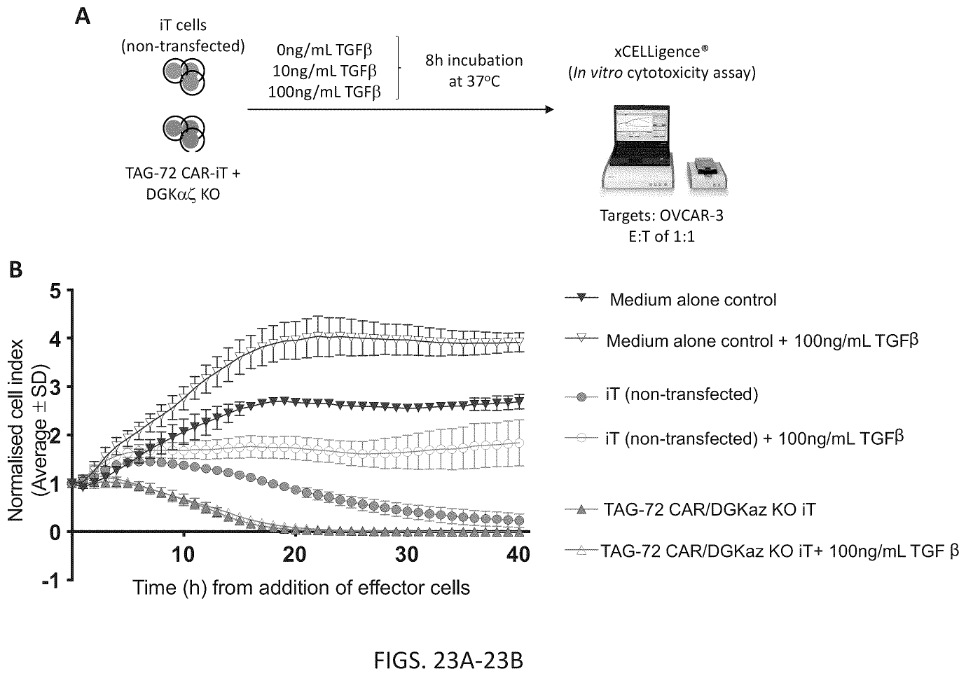

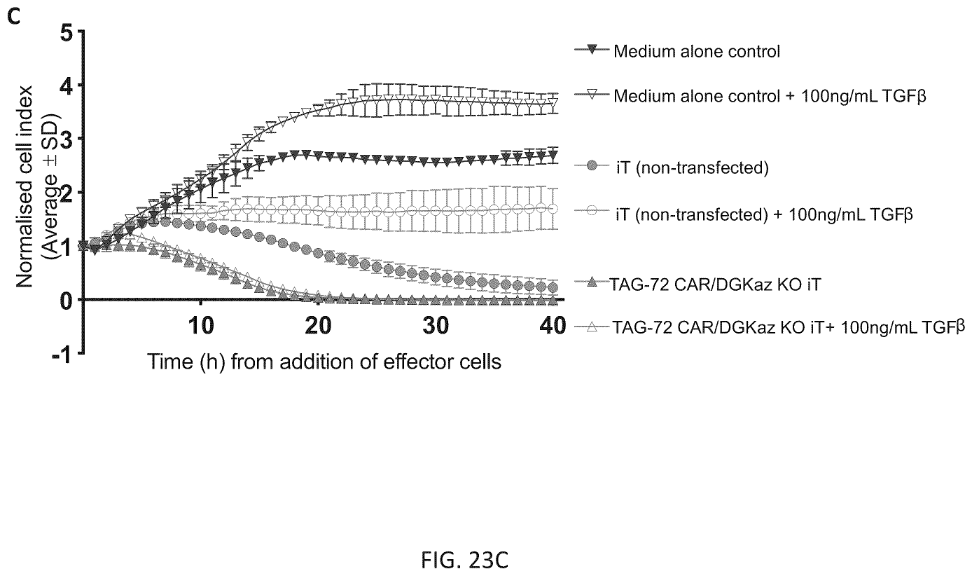

The functional advantage of DGK KO in iT cells in the tumour microenvironment was modelled using in vitro cytotoxic functional assays (as previously outlined in Example 7) in the presence of TGFβ, as shown in FIG. 23A. In brief, iT cells with or without the CAR and DGKαζ KO were pre-conditioned with either 0 ng/mL, 10 ng/mL or 100 ng/mL TGFβ in optimal growth media for at least 8 h before employing the real-time cell monitoring system (xCELLigence®) to determine the killing efficiency of iT cells in vitro. Target cells at 10,000/100 μL (for example the ovarian cancer cell line OVCAR-3) were resuspended in culture media (for example, RPMI-1640 basal media) supplemented with 20% fetal calf serum and bovine insulin and deposited into a Real Time Cell Analysis microtitre ePlate compatible with the xCELLigence® system. Target cells were maintained at 37° C., 5% CO₂ for 3-10 h to allow for cellular attachment. Following attachment of target cells, iT effector cells with or without TAG-72 CAR and DGKαζ KO, and with or without exposure to TGFβ, were added at an E:T of 1:1. All co-cultures were maintained in optimal growth conditions TGFβ for at least 40 hrs. Further, target cells alone±TGFβ were maintained in parallel. Throughout, cellular impedance was monitored at 15 min intervals; a decrease in impedance is indicative of target cell detachment and ultimately cell death.

In the absence of TGFβ pre-conditioning, cytotoxic function is observed in iT (non-transfected) cells (FIG. 23B and FIG. 23C, closed symbols) where near complete target cell death, as indicated by a Normalised cell index of 0, is observed within the 40 h monitoring period. In contrast, pre-conditioning and maintenance of iT (non-transfected) cells in either 10 ng/mL TGFβ (FIG. 23B) or 100 ng/mL TGFβ (FIG. 23C) resulted in a reduction in in vitro cytotoxic function. Such inhibitory effects of TGFβ on in vitro cytotoxicity were not observed with TAG-72 CAR-iT cells with the DGKαζ KO, indicating that the deletion (knock-out) of DGKαζ made these cells less susceptible to the suppressive effects of TGFβ on the cytotoxic function of iT cells.

What is claimed is:

1. A method of generating stem cell-derived immune cells with enhanced function, comprising:
(I) modifying stem cells to inhibit the function of at least one target gene selected from the group consisting of DGKα and DGKζ;
wherein the modified stem cells are capable of differentiating into stem cell-derived immune cells that retain the target gene inhibition of the modified stem cells and comprise enhanced activity.

2. The method of claim 1, wherein the stem cells are pluripotent stem cells, selected from the group consisting of induced pluripotent stem cells (iPSCs) or embryonic stem cells.

3. The method of claim 1, wherein the stem cells are selected from the group consisting of pre-HSCs, hemogenic endothelium (HE), hematopoietic stem cells (HSCs).

4. The method of any of the preceding claims, wherein the stem cells are derived from a triple homozygous HLA haplotype donor.

5. The method of any of the preceding claims, further comprising selecting the modified stem cells obtained in step (I) wherein both alleles of the target gene are inhibited.

6. The method of claim 1 or claim 5, further comprising:
(i) contacting the modified stem cell obtained in step (I), or clonal cells generated from them, with a composition to obtain mesoderm cells;
(ii) contacting the mesoderm cells with a composition to obtain CD34+ cells; and
(iii) contacting the CD34+ cells with a composition to obtain stem cell-derived immune cells.

7. The method of any of the preceding claims, wherein said at least one target gene is DGKα.

8. The method of any of the preceding claims, wherein said at least one target gene is DGKζ.

9. The method of any of the preceding claims, wherein both DGKα gene and DGKζ gene are inhibited.

10. The method of any of the preceding claims, wherein the stem cell-derived immune cells are selected from multipotent progenitor cells, common lymphoid progenitor cells, early thymic progenitor cells, pre-T cell progenitor cells, pre-NK progenitor cells, T progenitor cells, NK progenitor cells, T cells, NK cells, NKT cells, B cells, common myeloid progenitor cells, macrophages and monocytes.

11. The method of any of the preceding claims, wherein the immune cells are T cells expressing at least one of the markers selected from CD2, CD5, CD7, CD4, CD8a, CD8b, CD3, TCRαβ and TCRγδ.

12. The method of any of the preceding claims, wherein the immune cells are NK cells expressing CD56+ and CD45+.

13. The method of any of the preceding claims, wherein inhibition of the function of the target gene is achieved by a gene editing system.

14. The method of claim 13, wherein the gene editing system is selected from the group consisting of CRISPR/Cas, TALEN and ZFN.

15. The method of claim 13, wherein the gene editing system is a CRISPR/Cas system which comprises a guide RNA-nuclease complex.

16. The method of claim 15, wherein the guide RNA targets a nucleotide sequence selected from the group consisting of: SEQ ID NO: 1 to SEQ ID NO: 16.

17. The method of any one of claims 15-16, wherein the CRISPR/Cas system utilizes a guide RNA dependent nuclease selected from the group consisting of Cpf1, Cas1, Cas1B, Cas2, Cas3, Cas4, Cas5, Cas6, Cas7, Cas8, Cas9, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, and Csf4.

18. The method of any of the preceding claims, wherein the modified stem cells are further modified to comprise a nucleic acid encoding a chimeric antigen receptor (CAR).

19. The method of claim 18, wherein the modified stem cells express the CAR.

20. The method of claims 1-17, wherein the immune cells produced by the method are modified to comprise a nucleic acid encoding a chimeric antigen receptor (CAR).

21. The method of any of claims 18-20, wherein the immune cells produced by the method express the CAR.

22. The method of any of the preceding claims, wherein the immune cells produced by the method recognize one or more target antigens.

23. The method of any of the preceding claims, wherein the immune cells produced by the method recognize a tumor target or an infectious agent target.

24. The method of claim 22, wherein the target antigens are selected from the group consisting of TAG-72, CCR4, CD19, CD20, CD22, CD24, CD30, CD47, folate receptor alpha (FRα), BCMA, mesothelin, Muc1.

25. An immune cell produced by a method according to any one of claims 1-24.

26. A modified cell, wherein the function of at least one target gene selected from the group consisting of DGKα and DGKζ is inhibited, wherein the modified cell is capable of differentiating to an immune cell that retains the gene inhibition of the modified cell and comprises enhanced activity.

27. The modified cell of claim 26, wherein the cell is a stem cell selected from the group consisting of embryonic stem cells, umbilical cord stem cells and induced pluripotent stem cells.

28. The modified cell of claim 26, wherein the cell is selected from the group consisting of a hemogenic endothelium cell, hematopoietic progenitor cell, hematopoietic precursor cell, hematopoietic stem cell or hematopoietic-like stem cell.

29. The modified cell of any one of claims 26-28, wherein the cell is derived from a triple homozygous HLA haplotype donor.

30. The modified cell of any one of claims 26-29, wherein both alleles of the target gene are inhibited.

31. The modified cell of any one of claims 26-30, wherein said at least one target gene is DGKα.

32. The modified cell of any one of claims 26-30, wherein said at least one target gene is DGKζ.

33. The modified cell of any one of claims 26-30, wherein both DGKα and DGKζ genes are inhibited.

34. The modified cell of any one of claims 26-33, wherein the modified cell comprises a nucleic acid encoding a chimeric antigen receptor (CAR).

35. The modified cell of claim 34, wherein the modified cell expresses the CAR.

36. A composition for modifying a cell to inhibit the function of at least one target gene selected from the group consisting of DGKα and DGKζ comprising: a guide RNA-nuclease complex capable of editing the sequence of a target gene, wherein the guide RNA targets a nucleotide sequence is selected from the group consisting of: SEQ ID NO: 1 to SEQ ID NO: 16.

37. The composition of claim 36, wherein the nuclease comprises at least one protein selected from the group consisting of Cpf1, Cas1, Cas1B, Cas2, Cas3, Cas4, Cas5, Cas6, Cas7, Cas8, Cas9, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, and Csf4.

38. A method of treating a condition in a subject, comprising administering to the subject an immune cell according to claim 25.

39. The method of claim 38, wherein the condition is a cancer, an infection, an autoimmune disorder, fibrosis of an organ, or endometriosis.
